(19) United States
(12) Patent Application Publication
(10) Pub. No.: US 2015/0133653 A1
(43) Pub. Date: May 14, 2015

(54) COMPOUNDS AND BIOLOGICAL MATERIALS AND USES THEREOF

(71) Applicant: PHOTOBIOTICS LIMITED, LONDON (GB)

(72) Inventors: Gokhan Yahioglu, London (GB); Ioanna Stamati, London (GB); Mahendra Deonarain, London (GB)

(21) Appl. No.: 14/548,796

(22) Filed: Nov. 20, 2014

Related U.S. Application Data
(63) Continuation of application No. 13/257,083, filed on Dec. 6, 2011.

(30) Foreign Application Priority Data
Mar. 20, 2009 (GB) 0904825.7

(51) Int. Cl. C07D 487/22; A61K 41/00

(52) U.S. Cl. CPC C07D 487/22; A61K 41/0071
USPC 540/145

(57) ABSTRACT

The invention provides compounds of Formula I:

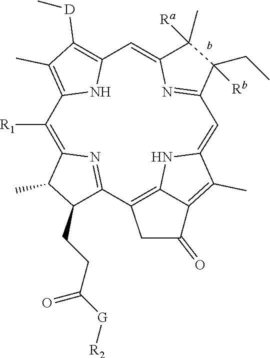

wherein b, D, R¹, R², G, R^{a} and R^{b} have meanings given in the description, or pharmaceutically-acceptable salts or solvates, or pharmaceutically functional derivatives thereof. The invention further provides process for conjugating the compounds to carrier molecules and uses of such compounds and conjugates in the treatment of disease.

### CROSS-REFERENCE TO RELATED APPLICATIONS

This is a continuation of U.S. application Ser. No. 13/257,083, filed Dec. 6, 2011, which is a national stage filing under 35 U.S.C. 371 of International Application No. PCT/GB2010/000518, filed Mar. 19, 2010, which claims foreign priority benefits to United Kingdom Patent Application No. 0904825.7, filed Mar. 20, 2009, which are incorporated herein by reference in their entireties.

The invention relates to improved compositions for photodynamic therapy (PDT) for the selective destruction of malignant, diseased, or infected cells or infective agents without causing damage to normal cells.

### BACKGROUND

Photodynamic Therapy (PDT) is a minimally invasive treatment for a range of conditions where diseased cells and tissues need to be removed [6,34,35]. Unlike ionising radiation, it can be administered repeatedly at the same site. Its use in cancer treatment is attractive because the use of conventional modalities such as chemotherapy, radiotherapy or surgery do not preclude the use of PDT and vice versa. PDT is also finding other applications where specific cell populations must be destroyed, such as blood vessels (in age-related macular degeneration (AMD [36]) or in cancer), the treatment of immune disorders [37], cardiovascular disease [38], and microbial infections [39,40].

PDT is a two-step or binary process starting with the administration of the photosensitiser (PS) drug, by intravenous injection, or topical application for skin cancer. The physico-chemical nature of the drug causes it to be preferentially taken up by cancer cells or other target cells [41]. Once a favourable tumour (or other target):normal organ ratio is obtained, the second step is the activation of the PS drug with a specific dose of light, at a particular wavelength. The photosensitizer, in its ground or singlet state absorbs a photon of light at a specific wavelength. This results in a short-lived excited singlet state. This can be converted by intersystem crossing to a longer-lived triplet state. It is this form of the sensitizer which carries out various cytotoxic actions.

The main classes of reactions are photooxidation by radicals (type I reaction), photooxidation by singlet oxygen (type II reaction), and photoreaction not involving oxygen (type III reaction). The triplet state form of the sensitiser causes the conversion of molecular oxygen found in the cellular environment into reactive oxygen species (ROS) primarily singlet oxygen (¹O₂) via a Type II reaction. If an activated photosensitizer interacts with cellular components, a Type I reaction occurs where electrons or protons are abstracted forming radicals such as hydroxyl radicals (OH. and superoxide (O₂⁻.). These molecular species cause damage to cellular components such as DNA, proteins and lipids [42]. A Type III mechanism has also been proposed where the triplet state photosensitier interacts with free radicals to cause cellular damage. The site of cellular damage depends upon the type of photosensitizer, length of incubation, type of cells and mode of delivery. Hydrophobic photosensitizers tend to damage cell membranes [42], whereas cationic photosensitizers localise within membrane vesicles such as mitochondria and cause damage there [43].

The light activation of ROS is highly cytotoxic. In fact some natural processes in the immune system utilise ROS as a way of destroying unwanted cells. These species have a short lifetime (<0.04 ms) and act in a short radius (<0.04 mm) from their point of origin. The destruction of cells leads to a necrotic-like area of tissue which eventually sloughs away or is resorbed. The remaining tissue heals naturally, usually without scarring. There is no tissue heating and connective tissue such as collagen and elastin are unaffected. This results in less risk to the underlying structures compared to thermal laser techniques, surgery or external beam radiotherapy. More detailed research has shown that PDT induces apoptosis (non-inflammatory cell death), and the resulting necrosis (inflammatory cell lysis) seen is due to the mass of dying cells which are not cleared away by the immune system [44,45].

PDT is a cold photochemical reaction, i.e. the laser light used is not ionising and delivers low levels of thermal energy, and PS drugs have very low systemic toxicity. The combination of PS drug and light result in low morbidity and minimal functional disturbance and offers many advantages in the treatment of diseases.

The newer generation of PS drugs have longer activation wavelengths thus allowing deeper tissue penetration by red light, higher quantum yield and better pharmacokinetics in terms of tumour selectivity and residual skin photosensitivity. These classes of PS drugs include the phthalocyanines, chlorins, texaphyrins and purpurins. The synthetic chlorin, Foscan™ is a very potent PS drug with a wavelength of activation of 652 nm, quantum yield of 0.43 and skin photosensitivity of about 2 weeks. There have been many clinical trials for a variety of cancers, with good results [35,36]. There are other PS drugs which have been developed and are in trials which can absorb at >700 nm, such as meta-tetrahydrophenyl bacteriochlorin (m-THPBC). A palladium-bacteriopheophorbide photosensitizer (TOOKAD) has been developed which shows promise in the treatment of prostate cancer with favourable, deep red absorption properties (763 nm absorption peak) [47].

Therefore, there are several advantages of PDT therapy. It offers non-invasive, low toxicity treatments which can be targeted by the light activation. The target cells cannot develop resistance to the cytotoxic species (ROS). Following treatment, little tissue scarring exists. However, PS drugs are not very selective for the target cells with target: blood ratios typically in single figures at best. In many situations this lack of selectivity leads to unacceptable damage to proximal normal tissues e.g. Photofrin™ [58, 59] in oesophageal cancers [60, 61], bladder cancer [62]. Because PS drugs “piggy-back” on blood proteins, they persist longer in the circulation than is desired, leaving the patient photosensitive for 2 weeks in the best of cases.

Unlike standard chemotherapeutics, photosensitiser drugs can still be active and functional while attached to carriers, as the cytotoxic effect is a secondary effect resulting from light activation. This makes them amenable to specific drug delivery mechanisms involving conjugation to targeting molecules.

Currently, the preferred approach to link photosensitizer drugs to targetable elements is the direct conjugation of derivatized photosensitizer drugs to whole monoclonal antibodies. Whole antibodies have a molecular weight of 150 KDa, resulting in very large photo-immunoconjugates with unfavourable pharmacokinetics, such as poor tumour: organ ratios (2:1) [63,64] which take a long time to achieve. Current literature suggest that photosensitizer drugs linked to residues on a monoclonal antibody can have a detrimental effect on each other, with quenching effects occurring due to poor spectroscopic properties [65]. In addition to this, it has been shown that poor, and unreliable, loading of photosensitizer onto the antibody is seen with ratios of 4:1 being typical before the antibody aggregates or loses function [63-69].

Coupling of photosensitisers has been tried using various strategies with various monoclonal antibodies. For example PPa has been coupled to anti-Her 2 monoclonal antibodies. In order to achieve good sensitiser: antibody coupling ratios (in the region of 10:1) the antibody had to be made more soluble by attaching chains of polyethylene glycol [68]. This PEGylation would have a detrimental effect on the conjugate pharmacokinetics resulting in poorer tumour:blood ratios. Furthermore, non-covalent binding of photosensitiser to antibody was also seen here. Such non-covalent binding has been a feature of most reported attempts to produce antibody-photosensitiser conjugates, and represents a major problem in reliably producing well characterised conjugates. In a further study, a porphyrin sensitiser was used with monoclonal antibodies 17.1A, FSP77 and 35A7 using a isothiocyanate coupling method resulting in sensitiser:antibody ratio no better than 2.8:1 [67]. Another example was verteporfin (benzoporphyrin derivative, BPD) with monoclonal antibody C225 (anti-EGFR). Here, coupling ratios of greater than 11:1 resulted in poor immunoreactivity and solubility [69]. The best ratios were about 7:1. These examples serve to illustrate the problems of producing well characterised conjugates with high photosensitiser:antibody ratios, and suggest that the use of fragments which are one third to one sixth smaller than whole antibodies would be even less successful given the solubility and loading problems seen with the larger protein species.

The work on PS drugs attached to monoclonal antibodies has shown that if too many PS molecules are attached to an individual monoclonal antibody the hydrophobicity can be affected and an adverse effect on the pharmacokinetics may result. Given the problems with whole monoclonal antibodies, it is widely believed that small fragments (such as a scFv, 30 KDa) would have very unfavourable coupling efficiencies, resulting in only one or two photosensitisers being coupled. A general review of the techniques involved in the synthesis of antibody fragments which retain their selective binding sites is to be found in Holliger and Hudson, Nature Biotechnology (2005) 23, 1126-36. Birchler et al [70] attempted to produce an effective scFv—photosensitiser conjugate but were only able to couple a single photosensitizer through a site-specific cysteine residue to a scFv.

Other groups have tried to circumvent these problems by attempting to link PS drugs to designated ‘carriers’ such as branched carbohydrate [71] or polyethylene glycol chains [72] and poly-lysine [73] chains. These approaches all require additional conjugation steps as the ligand-carriers cannot be made entirely recombinantly. Using such polymers may also have problems such as proteolyic instability in vivo. It is known that when photosensitizers are attached in this way, they self-quench, destroying their photophysical properties and rely on degradation in lysozymes to ‘de-quench’ before they can become active photosensitizers [71]. Therefore, higher coupling ratios can be achieved, up to 10:1, but only with lower phototoxicity and lower singlet oxygen yield than that obtained with free (un-coupled) photosensitizer. Studies by Roder et al. [71] showed that the photosensitising activity of pheophorbides when covalently linked in large numbers around the periphery of a dendrimer were dramatically reduced. This is a result of energy transfer processes, mainly Forster energy transfer from dye to dye. Forster transfer is distance dependant and drops off rapidly with distance. The interaction of dye molecules leads to changes in the absorption spectrum, reduced fluorescence lifetimes and singlet oxygen quantum yields. Fusion proteins combining an antibody fragment with a protein carrier molecule have also been described by our group [74].

Glickman et al [75,76] describe monoclonal antibody targeted PDT against the VEGF vasculature target for ocular disease. This uses standard coupling conditions with no description of antibody: photosensitizer ratios. However Hasan et al [77] discloses a two-solvent system to improve upon the photosensitizer: antibody coupling ratios. Here, using very high concentrations of organic solvents (typically 40-60%) mixed with aqueous buffers, ratios of up to 11:1 have been reported. However, the high concentrations of solvent used are unlikely to be tolerated by all antibodies. No mention is made of using fragments, but given their greater sensitivity to organic solvents, they would not be expected to be viable in this method. Also in Hasan et al [77], the large number of coupled photosensitizers are self-quenching, hence this system relies upon internalisation and lysozomal degradation to release phototoxic molecules. Photo-immunoconjugates bound to the cell surface are not expected to be exposed to degradation enzymes like those found in intracellular lysozomes. This may exclude the targeting of low/non-internalizing antigens such as CEA and matrix/stromal antigens.

By linking novel or established PS drugs to small, targetable carrier proteins, it is possible to deliver a highly specific dose of PS drug to a target tissue, which can later be activated by light. These carrier-PS drug conjugates have advantages over existing targeted and non-targeted PDT approaches in that a greater amount of PS drug can accumulate in the target tissue, with tissue to blood/normal organ ratios of 20:1 or better, in shorter time intervals. Additionally, these agents could have advantages over other targetable strategies with little or no immunogenicity and lower side effects. Smaller ligands have been used to deliver photosensitizers, such as insulin [78], transferrin [79,80], albumin [81], annexins [82], toxins [83], estrogen [84], rhodamine derivatives [85], folate [86] and growth factors such as EGF [87] and VEGF [88].

WO 2007/042775 describes a method for coupling photo-sensitisers to biological targeting proteins such as antibody fragments (e.g. scFvs) using optimised coupling conditions to ensure that the carrier remains functional and soluble. The conjugates described possess a high and consistent molar ratio of covalently attached photosensitisers without non-covalent binding. WO 2007/042775 also describes engineered recombinant antibody-photosensitiser conjugates with optimised photophysical and photodynamic properties, and methods to produce them. Furthermore WO 2007/042775 describes ways of coupling other ‘non-photosensitising’ molecules which enhance the photo-physical and photodynamic properties of the overall conjugate.

The biological nature of antibodies requires that they be maintained in mostly aqueous buffers in order to retain function and integrity. However, photosensitizers tend to be hydrophobic in nature and are poorly soluble in the buffer conditions normally used for antibodies. Coupling a photosensitizer to an antibody under aqueous conditions will result in poor photosensitizer: antibody ratios and in solvents will result in damaged antibody proteins. WO 2007/042775 describes a method utilizing a combination of organic solvents at low concentration.

The problem of producing photoimmunoconjugates (PIC's) of high purity and potency for targeted photodynamic therapy (PDT) has not been solved in the art.

The large hydrophobic face of a porphyrinic macrocycle (as found on photosensitiser molecules) represents a challenge to water solubilisation as the choice of bio-conjugatable group must enable conjugation to be carried out without interference with the functional group that affords water solubility. The inventors' work with the more soluble sulphonic acid active ester derivative of Pyropheophorbide-a (PPa) (Bhatti M, Yahioglu G, Milgrom L R, Garcia-Maya M, Chester K A, Deonarain M P. Int J. Cancer. 2008 Mar. 1; 122(5):1155-63) has shown that cofacial interaction between the hydrophobic porphyrinic macrocycle is a great problem and is a likely mechanism for aggregation, excited state quenching and precipitation in aqueous solutions. Aggregation and precipitation of the photosensitiser drastically reduces the efficiency of conjugation.

Chlorins like pyropheophorbide-a and bacteriochlorins like TOOKAD (see Chart 1) absorb strongly in the red and near-infrared regions, respectively (Advances in Photodynamic Therapy, Basic Translational and Clinical', Editors: M R Hamblin and P Mroz, Published by Artech House, USA, 2008). However, water-soluble derivatives of such naturally occurring chlorins and bacteriochlorins have not been readily available.

Chlorin e6 is a commercially accessible derivative of chlorophyll a containing three ionisable carboxylic acid groups, the aspartyl derivative of which is the only water-soluble chlorin derivative in current development as a stand alone photosensitiser (Taloporfin sodium, see Chart 1). However, the presence of substituents at nearly all the other peripheral positions of the macrocycle makes synthetic manipulation difficult especially the introduction of a potential handle for conjugation. This is the same problem encountered when dealing with PPa which has a single propionic acid side-chain which is available for activation and conjugation to amine residues like lysines on various antibody formats but whose full complement of substituents about the perimeter of the macrocycle severely limits synthetic malleability (see Chart 1).

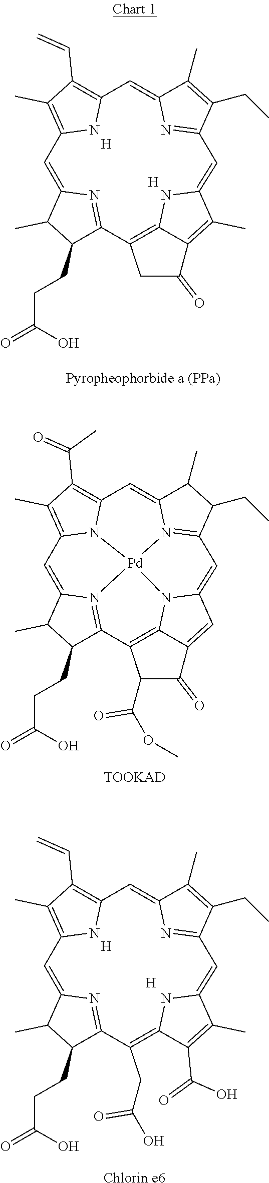

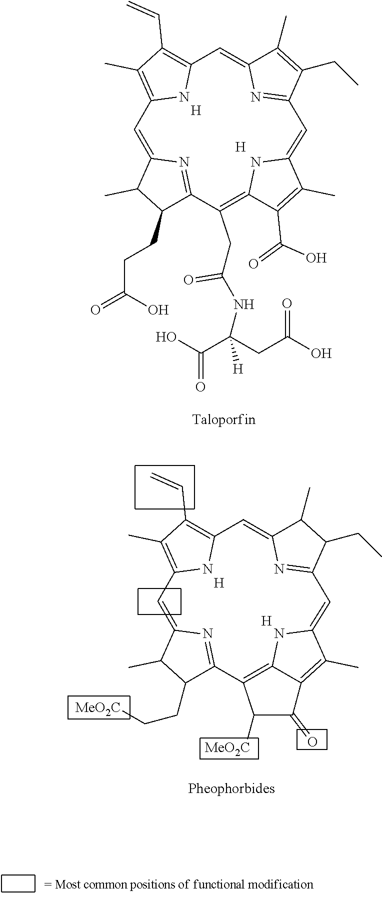

Derivitisation Positions for the Pheophorbides

The two most common approaches for functionalising PPa are modification of the vinyl group through oxidation/addition or alkylation of the propionic acid side chain (or a combination of both). In the last thirty years a large number of derivatives have been prepared and reported for numerous applications. The most common positions of functional modification are shown in the diagram above. However, there is a lack of compounds in the prior art that incorporate both design features of a group imparting high water solubility and a bioconjugatable handle, namely a free carboxylic acid functional group.

A third approach for functionalising PPa involves functionalising the 5-meso position. This has remained rarely used since the first report of 5-bromination on methyl PPa where the double bond of the vinyl group has been reduced, nearly thirty years ago (G W Kenner, S W McCombie and K Smith, J C S Perkin Trans 1, 1973, 2517).

Recently, Wasielewski and co-workers (R F Kelley, M J Tauber and M R Wasielewski, Angew. Chemie. Int. Ed. 2006, 45, 7979) have shown that one can carry-out metal catalysed cross-coupling chemistry on this meso-5-bromo derivative of methyl PPa, enabling the introduction of a limited number of alkyl and aryl groups into this sterically hindered position.

In the present invention, this technology is greatly expanded to enable the introduction of either a wide variety of peripheral functional groups to impart both water solubility and minimise non-covalent binding, or to attach a “handle” for conjugation to a carrier.

The present invention describes new compounds suitable for use as photosensitisers, which have greater solubility in aqueous solutions. The new photosensitisers act to suppress co-facial attraction and reduce non-covalent binding to proteins. The present invention also describes processes for making new photoimmunoconjugates (PICs) with the new photosensitisers that demonstrate improved in vitro activity, improved pharmacokinetics and improved in vivo activity.

### SUMMARY OF THE INVENTION

The present invention discloses a series of novel derivatives of PPa. These hydrophobic photosensitisers have been developed for improved water solubility, drug efficacy and improved conjugation to proteins/peptides. The approach involves the synthesis of a number of key intermediates which allow the preparation of porphyrins, chlorins and bacteriochlorins bearing a single amine or thiol reactive group and water solubilising groups, which both act to suppress co-facial interaction (a likely mechanism for aggregation and precipitation in aqueous buffer) and reduce non-covalent binding to proteins.

In a first aspect the present invention provides a compound of Formula I:

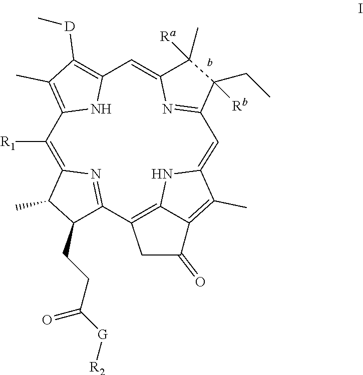

wherein:
when b represents a double bond, D represents —CH₂— or Q, R^{a} and R^{b} are both not present;
when b represents a single bond, D represents —C(O)—, —CH₂— or Q, R^{a} and R^{b} are either both H or both —OH;
when R₁ represents H or a moiety containing a functional group that can react with carboxyl, hydroxyl, amino or thiol group,
R₂ represents H or a solubilising group; or
when R₁ represents H or a solubilising group,
R₂ represents H or a moiety containing a functional group that can react with carboxyl, hydroxyl, amino or thiol group;
G represents O or a direct bond;
Q represents a structural fragment of formula Ig or Ih,

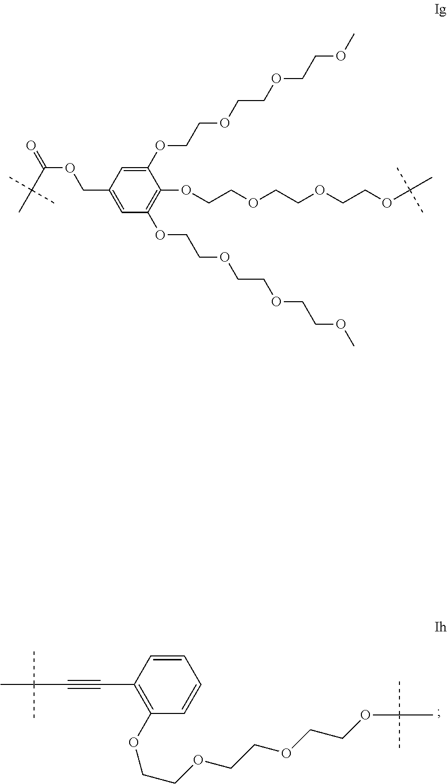

or a pharmaceutically-acceptable salt or solvate, or a pharmaceutically functional derivative thereof.

By “a moiety containing a functional group that can react with carboxyl, hydroxyl, amino or thiol group” we mean a moiety that is or, preferably, a moiety that contains: a halo or, preferably, a carboxyl, mercapto, amino, haloalkyl, phosphoramidityl, N-succinimidyl ester, sulfo-N-hydroxy succinimidyl ester, fluorophenyl esters, isothiocyanato, iodoacetamidyl or a maleimidyl group. It is envisaged that such a group may be suitable for use as a handle for conjugation to a suitable carrier molecule.

By “a solubilising group” we mean any functional group that increases the solubility of the entire compound in water and can be cationic (e.g. a group containing one or more pyridinium salts), anionic (a group containing one or more salts of carboxylic acids) or neutral (e.g. a group containing one or more oligo- or polyethylene glycol groups). It is envisaged that such group may act to reduce co-facial interaction of the compounds of Formula I in solution, and prevent intermolecular aggregation and precipitation.

For the avoidance of doubt, it is envisaged that, in some circumstances, “a moiety containing a functional group that can react with carboxyl, hydroxyl, amino or thiol group” can also fall under the definition of “a solubilising group”, and vice-versa.

Pharmaceutically-acceptable salts that may be mentioned include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula I in the form of a salt with another counter-ion, for example using a suitable ion exchange resin.

Examples of pharmaceutically acceptable addition salts include those derived from mineral acids, such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

“Pharmaceutically functional derivatives” of compounds of formula I as defined herein includes ester derivatives and/or derivatives that have, or provide for, the same biological function and/or activity as any relevant compound. Thus, for the purposes of this invention, the term also includes prodrugs of compounds of formula I.

The term “prodrug” of a relevant compound of formula I includes any compound that, following oral or parenteral administration, is metabolised in vivo to form that compound in an experimentally-detectable amount, and within a predetermined time (e.g. within a dosing interval of between 6 and 24 hours (i.e. once to four times daily)). For the avoidance of doubt, the term “parenteral” administration includes all forms of administration other than oral administration.

Prodrugs of compounds of formula I may be prepared by modifying functional groups present on the compound in such a way that the modifications are cleaved, in vivo when such prodrug is administered to a mammalian subject. The modifications typically are achieved by synthesizing the parent compound with a prodrug substituent. Prodrugs include compounds of formula I wherein a hydroxyl, amino, sulfhydryl, carboxy or carbonyl group in a compound of formula I is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. I-92, Elesevier, New York-Oxford (1985).

Compounds of formula I, as well as pharmaceutically-acceptable salts, solvates and pharmaceutically functional derivatives of such compounds are, for the sake of brevity, hereinafter referred to together as the “compounds of formula I”.

Compounds of formula I contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

It is envisaged that, where b is a single bond and R^{a} and R^{b} are both OH, the OH groups in question can be in the trans or, preferably, cis orientation with respect to each other.

For the avoidance of doubt, in respect of structural fragments Ig and Ih, it is envisaged that the dashed line on the left-hand side of the molecule as represented herein (i.e. the dashed line at the —C(O)— terminus of the fragment of formula Ig and the dashed line at the alkyne terminus of the fragment of formula Ih) denotes the point of attachment to the central ring (i.e. the porphyrin ring) of the compound of formula I.

Compounds of formula I that may be mentioned include those in which R₁ represents H or a moiety containing a functional group that can react with carboxyl, hydroxyl, amino or thiol group and R₂ represents H or a solubilising group.

Compounds of formula I that may be mentioned include those in which R₁ represents H or a solubilising group and R₂ represents H or a moiety containing a functional group that can react with carboxyl, hydroxyl, amino or thiol group.

Compounds of formula I that may be mentioned include those in which:

R₁ represents H, —(CH₂)ₜ—X, —(CH₂)ₐ—C(R₃)═C(R₄)—(CH₂)ᵥ—X, or —(CH₂)_{w}—C≡C—(CH₂)ₓ—X;

t represents 1 to 20 (e.g. 1 to 12);

the sum of u and v is from 2 to 6;

the sum of w and x is from 2 to 15 (e.g. 2 to 10);

X represents —C(O)-L₁, —OH, a sulfonyl ester (e.g. mesylate, tosylate), —NO₂, —CHO, —N₃, —CN, —SH, —NHR₃ₐ, halo, phosphoramidityl, N-hydroxy succinimidyl ester, sulfo-N-hydroxy succinimidyl ester, fluorophenyl esters (e.g. 1,2,3,5,6-pentafluorophenyl, 4-sulfo-2,3,5,6-pentafluorophenyl), isothiocyanato, iodoacetamidyl, maleimidyl, aryl or hetroaryl (which latter two groups are substituted by one or more groups selected from —C(O)-L₁, —OH, a sulfonyl ester (e.g. mesylate, tosylate), —NO₂, —CHO, —N₃, —CN, —SH, —NHR₃ₐ, halo, phosphoramidityl, N-hydroxy succinimidyl ester, sulfo-N-hydroxy succinimidyl ester, fluorophenyl esters, isothiocyanato, iodoacetamidyl and maleimidyl);

L₁ represents —OH or a suitable leaving group (e.g.—O—C(O)—R₅, halo, an activated ester such as 1-oxybenzotriazoyl or an aryloxy group optionally substituted with one or more subsistent selected from nitro, fluoro, chloro, cyano and trifluoromethyl) or —C(O)-L₁ represents a carboxylic acid functional group activated by a carbodiimide;

R₂ represents H, alkyl, cycloalkyl, alkylenyl, alkynyl, aryl, benzyl, heteroaryl (wherein the latter three groups may be substituted by one or more groups selected from —OH, —NH₂ or a C1 to C6 alkyl substituted by one or more halo atoms) independently substituted by one or more —C(O)O⁻E⁺ groups, —SO₃⁻E⁺ groups, a quarternary ammonium salt, a pyridinium ion or linear or branched oligo or poly-ethyleneoxy groups (wherein the total number of oligo or poly-ethyleneoxy groups is from 2 to 100 (e.g. about 3 to about 20)),

or R₂ represents —NR₆(R₇) or —N(R₆ₐ)—(CH₂)—SO₃⁻E⁺;

R₃ to R₅ and R₃ₐ independently represent C1 to C6 alkyl optionally substituted by one or more groups selected from —OH and halo;

R₆ and R₇ independently represent H, alkynyl, a pyridinium ion, —(CH₂)_{z}—NR₈(R₉) or —(CH₂)_{z}—N⁺R₈(R₉)(R₁₀) A⁻, provided that at least one of R₆ and R₇ is not H;

R₆ₐ represents H or C1 to C6 alkyl optionally substituted with one or more groups selected from —OH and halo;

z represents 1 to 20 (e.g. 1 to 10);

R₈ to R₁₀ independently represents H, alkyl, alkenyl, alkynyl, aryl or heteroaryl optionally substituted by one or more groups selected from —OH and halo;

E⁺ represents a suitable cationic group (e.g. Na⁺, K⁺);

A⁻ represents a suitable anionic group (e.g. I⁻, Cl⁻, Br⁻).

Compounds of formula I that may be mentioned include those in which:

when b represents a double bond, D represents —CH₂—, R^{a} and R^{b} are both not present;

when b represents a single bond, D represents —C(O)— or —CH₂—, R^{a} and R^{b} are both H;

G represents 0;

R₁ represents —(CH₂)ₜ—X, —(CH₂)ᵤ—C(R₃)═C(R₄)—(CH₂)ᵥ—X, or —(CH₂)_{w}—C≡C—(CH₂)ₓ—X;

t represents 1 to 20 (e.g. 1 to 12);

the sum of u and v is from 2 to 6;

the sum of w and x is from 2 to 15 (e.g. 2 to 10);

X represents —C(O)-L₁, —OH, a sulfonyl ester (e.g. mesylate, tosylate), —NO₂, —CHO, —N₃, —CN, —SH, —NHR₃ₐ, halo, phosphoramidityl, N-hydroxy succinimidyl ester, sulfo-N-hydroxy succinimidyl ester, fluorophenyl esters (e.g. 1,2,3,5,6-pentafluorophenyl, 4-sulfo-2,3,5,6-pentafluorophenyl), isothiocyanato, iodoacetamidyl, maleimidyl, aryl or hetroaryl (which latter two groups are substituted by one or more groups selected from —C(O)-L₁, —OH, a sulfonyl ester (e.g. mesylate, tosylate), —NO₂, —CHO, —N₃, —CN, —SH, —NHR₃ₐ, halo, phosphoramidityl, N-hydroxy succinimidyl ester, sulfo-N-hydroxy succinimidyl ester, fluorophenyl esters, isothiocyanato, iodoacetamidyl and maleimidyl);

L₁ represents —OH or a suitable leaving group (e.g. —O—C(O)—R₅, halo, an activated ester such as 1-oxybenzotriazoyl or an aryloxy group optionally substituted with one or more subsistent selected from nitro, fluoro, chloro, cyano and trifluoromethyl) or —C(O)-L₁ represents a carboxylic acid functional group activated by a carbodiimide;

R₂ represents alkyl, cycloalkyl, alkylenyl, alkynyl, aryl, benzyl, heteroaryl (wherein the latter three groups may be substituted by one or more groups selected from —OH, —NH₂ or a C1 to C6 alkyl substituted by one or more halo atoms) independently substituted by one or more —C(O)O⁻E⁺ groups, —SO₃⁻E⁺ groups, a quarternary ammonium salt, a pyridinium ion or linear or branched oligo or poly-ethyleneoxy groups (wherein the total number of oligo or poly-ethyleneoxy groups is from 2 to 100 (e.g. about 3 to about 20)), or R₂ represents —NR₆(R₇) or —N(R₆ₐ)—(CH₂)—SO₃⁻E⁺;

R₃ to R₅ and R₃₂ independently represent C1 to C6 alkyl optionally substituted by one or more groups selected from —OH and halo;

R₆ and R₇ independently represent H, a pyridinium ion, —(CH₂)_{z}—NR₈(R₉) or —(CH₂)_{z}—N⁺R₈(R₉)(R₁₀)A⁻, provided that at least one of R₆ and R₇ is not H;

R₆ₐ represents H or C1 to C6 alkyl optionally substituted with one or more groups selected from —OH and halo;

z represents 1 to 20 (e.g. 1 to 10);

R₈ to R₁₀ independently represents H, alkyl, alkenyl, alkynyl, aryl or heteroaryl optionally substituted by one or more groups selected from —OH and halo;

E⁺ represents a suitable cationic group (e.g. Na⁺, K⁺);

A⁻ represents a suitable anionic group (e.g. I⁻, Cl⁻, Br⁻).

Compounds of Formula I that may be mentioned include those in which D represents Q, and Q represents a structural fragment of formula Ih or, particularly, Ig.

Further compounds of Formula I that may be mentioned include:

(a) b represents a double bond, D represents Q, Q represents a structural fragment of formula Ig, G represents O, R₁ represents —(CH₂)_{w}—C≡C—(CH₂)ₓ—X, R₂ represents alkyl;

(b) b represents a double bond, D represents Q, Q represents a structural fragment of formula Ig, G represents O, R₁ represents —(CH₂)_{w}—C≡C—(CH₂)ₓ—X, R₂ represents benzyl substituted by branched poly-ethyleneoxy groups;

(c) b represents a double bond, D represents Q, Q represents a structural fragment of formula Ih, G represents O, R₁ represents H, R₂ represents H;

(d) b represents a double bond, D represents —CH₂—, G represents a direct bond, R₁ represents halo (particularly, Br), R₂ represents —NR₆(R₇), R₆ represents alkynyl, R₇ represents H;

(e) b represents a single bond, R^{a} and R^{b} both represent —OH, D represents —CH₂—, G represents O, R₁ represents halo (particularly, Br), R₂ represents benzyl substituted by branched poly-ethyleneoxy groups.

In a yet further aspect the present invention provides a compound of Formula III:

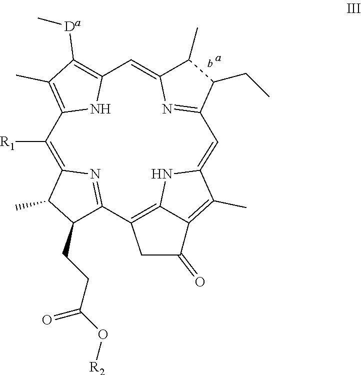

wherein:

R¹, R² are as defined herein;

when b^{a} represents a double bond, D^{a} represents —CH₂—;

when b^{a} represents a single bond, D^{a} represents —C(O)— or —CH₂—,

or a pharmaceutically-acceptable salt or solvate, or a pharmaceutically functional derivative thereof.

Compounds of formula III, as well as pharmaceutically-acceptable salts, solvates and pharmaceutically functional derivatives of such compounds are, for the sake of brevity, hereinafter referred to together as the “compounds of formula III”.

Compounds of formula III that may be mentioned include those in which R₁ represents a moiety containing a functional group that can react with carboxyl, hydroxyl, amino or thiol group and R₂ represents a solubilising group.

Compounds of formula III that may be mentioned include those in which R₁ represents a solubilising group and R₂ represents a moiety containing a functional group that can react with carboxyl, hydroxyl, amino or thiol group.

Compounds of formula III that may be mentioned include those in which:

R₁ represents —(CH₂)ₜ—X, —(CH₂)_{U}—C(R₃)═C(R₄)—(CH₂)ᵥ—X, or —(CH₂)_{w}—C≡C—(CH₂)ₓ—X;

t represents 1 to 20 (e.g. 1 to 12);

the sum of u and v is from 2 to 6;

the sum of w and x is from 2 to 15 (e.g. 2 to 10);

X represents —C(O)-L₁, —OH, a sulfonyl ester (e.g. mesylate, tosylate), —NO₂, —CHO, —N₃, —CN, —SH, —NHR₃ₐ, halo, phosphoramidityl, N-hydroxy succinimidyl ester, sulfo-N-hydroxy succinimidyl ester, fluorophenyl esters (e.g. 1,2,3,5,6-pentafluorophenyl, 4-sulfo-2,3,5,6-pentafluorophenyl), isothiocyanato, iodoacetamidyl, maleimidyl, aryl or hetroaryl (which latter two groups are substituted by one or more groups selected from —C(O)-L₁, —OH, a sulfonyl ester (e.g. mesylate, tosylate), —NO₂, —CHO, —N₃, —CN, —SH, —NHR₃ₐ, halo, phosphoramidityl, N-hydroxy succinimidyl ester, sulfo-N-hydroxy succinimidyl ester, fluorophenyl esters, isothiocyanato, iodoacetamidyl and maleimidyl);

L₁ represents —OH or a suitable leaving group (e.g.—O—C(O)—R₅, halo, an activated ester such as 1-oxybenzotriazoyl or an aryloxy group optionally substituted with one or more subsistent selected from nitro, fluoro, chloro, cyano and trifluoromethyl) or —C(O)-L₁ represents a carboxylic acid functional group activated by a carbodiimide;

R₂ represents alkyl, cycloalkyl, alkylenyl, alkynyl, aryl, benzyl, heteroaryl (wherein the latter three groups may be substituted by one or more groups selected from —OH, —NH₂ or a C1 to C6 alkyl substituted by one or more halo atoms) independently substituted by one or more —C(O)O⁻E⁺ groups, —SO₃⁻E⁺ groups, a quarternary ammonium salt, a pyridinium ion or linear or branched oligo or poly-ethyleneoxy groups (wherein the total number of oligo or poly-ethyleneoxy groups is from 2 to 100 (e.g. about 3 to about 20)), or R₂ represents —NR₆(R₇) or —N(R₆ₐ)—(CH₂)—SO₃⁻E⁺;

R₃ to R₅ and R₃ₐ independently represent C1 to C6 alkyl optionally substituted by one or more groups selected from —OH and halo;

R₆ and R₇ independently represent H, a pyridinium ion, —(CH₂)_{z}—NR₈(R₉) or —(CH₂)_{z}—N⁺R₈(R₉)(R₁₀)A⁻ provided that at least one of R₆ and R₇ is not H;

R₆ₐ represents H or C1 to C6 alkyl optionally substituted with one or more groups selected from —OH and halo;

z represents 1 to 20 (e.g. 1 to 10);

R₈ to R₁₀ independently represents H, alkyl, alkenyl, alkynyl, aryl or heteroaryl optionally substituted by one or more groups selected from —OH and halo;

E⁺ represents a suitable cationic group (e.g. Na⁺, K⁺);

A⁻ represents a suitable anionic group (e.g. I⁻, Cl⁻, Br⁻).

It is intended that references to D hereinafter will also apply to D^{a}. Similarly, references to b hereinafter will also apply to b^{a}.

Compounds of formula I or formula III that may be mentioned include those in which: R₁ represents a structural fragment of formula Ia, Ib, Ic, Id, Ie, If,

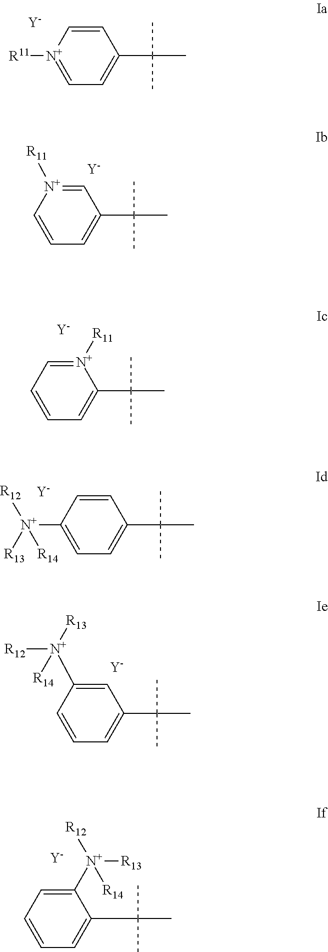

wherein the dashed lines indicate the point of attachment to the rest of the molecule, or

R₁ represents —(CH₂)ₜ—Z, —(CH₂)ₐ—C(R₃)═C(R₄)—(CH₂)ᵥ—Z, or —(CH₂)_{w}—C≡C—(CH₂)ₓ—Z;

R¹¹ represents H, alkyl (optionally substituted by one or more groups selected from —OH, halo and linear or branched ethyleneoxy groups (wherein the total number of oligo or poly-ethyleneoxy groups is from 2 to 100 (e.g. about 3 to about 20)), or linear or branched ethyleneoxy groups (wherein the total number of oligo or poly-ethyleneoxy groups is from 2 to 100 (e.g. about 3 to about 20));

R₁₂ to R₁₄ independently represent H or C1 to C6 alkyl optionally substituted by one or more groups selected from —OH, halo and linear or branched ethyleneoxy groups (wherein the total number of oligo or poly-ethyleneoxy groups is from 2 to 100 (e.g. about 3 to about 20));

Y⁻ represents any suitable anionic group (e.g. I⁻, Br⁻, Cl⁻);

t represents 1 to 20 (e.g. 1 to 12);

the sum of u and v is from 2 to 6;

the sum of w and x is from 2 to 15 (e.g. 2 to 10);

Z represents —C(O)O⁻E⁺, —SO₃⁻E⁺, a quarternary ammonium salt, a structural fragment of formulae Ia to If, or Z represents aryl, benzyl, heteroaryl (wherein the latter three groups may be substituted by one or more groups selected from —OH, —NH₂ or a C1 to C6 alkyl substituted by one or more halo atoms) substituted by one or more —C(O)O⁻E⁺ groups, —SO₃⁻E⁺ groups, a quarternary ammonium salt, a pyridinium ion or linear or branched oligo or poly-ethyleneoxy groups (wherein the total number of oligo or poly-ethyleneoxy groups is from 2 to 100 (e.g. about 3 to about 20));

E⁺ represents any suitable cation (e.g. Na⁺, K⁺);

R₂ represents —C(O)-L₃, —OH, a sulfonyl ester (e.g. mesylate, tosylate), —NO₂, —CHO, —N₃, —CN, —SH, —NHR₃ₐ, halo, phosphoramidityl, N-hydroxy succinimidyl ester, sulfo-N-hydroxy succinimidyl ester, fluorophenyl esters (e.g. 1,2,3,5,6-pentafluorophenyl, 4-sulfo-2,3,5,6-pentafluorophenyl), isothiocyanato, iodoacetamidyl, maleimidyl, aryl or hetroaryl (which latter two groups are substituted by one or more groups selected from —C(O)-L₁, —OH, a sulfonyl ester (e.g. mesylate, tosylate), —NO₂, —CHO, —N₃, —CN, —SH, —NHR₃ₐ, halo, phosphoramidityl, N-hydroxy succinimidyl ester, sulfo-N-hydroxy succinimidyl ester, fluorophenyl esters, isothiocyanato, iodoacetamidyl and maleimidyl);

L₃ represents —OH or a suitable leaving group (e.g. —O—C(O)—R₁₅, halo, an activated ester such as 1-oxybenzotriazoyl or an aryloxy group optionally substituted with one or more subsistent selected from nitro, fluoro, chloro, cyano and trifluoromethyl) or —C(O)-L₁ represents a carboxylic acid functional group activated by a carbodiimide; and

R₁₅ represents C1 to C6 alkyl optionally substituted by one or more groups selected from —OH and halo.

Unless otherwise stated, the term “alkyl” refers to an unbranched or branched, cyclic, saturated or unsaturated (so forming, for example, an alkenyl or alkynyl)hydrocarbyl radical, which may be substituted or unsubstituted (with, for example, one or more halo atoms). Where the term “alkyl” refers to an acyclic group, it is preferably C₁₋₂₀ alkyl (e.g. C₁₋₁₂) and, more preferably, C₁₋₆ alkyl (such as ethyl, propyl, (e.g. n-propyl or isopropyl), butyl (e.g. branched or unbranched butyl), pentyl or, more preferably, methyl). Where the term “alkyl” is a cyclic group (which may be where the group “cycloalkyl” is specified), it is preferably C₃₋₁₂ cycloalkyl and, more preferably, C₆₋₁₀ (e.g. C₆₋₇) cycloalkyl. For the avoidance of doubt, the term “alkenyl” when used herein refers to an alkyl group as hereinbefore defined containing at least two carbons and at least one carbon-carbon double bond and the term “alkynyl” when used herein refers to an alkyl group as hereinbefore defined containing at least two carbons and at least one carbon-carbon triple bond.

The terms “halo” and/or “halogen”, when used herein, include fluorine, chlorine, bromine and iodine.

The term “aryl” when used herein includes C₆₋₁₄ (such as C₆₋₁₃ (e.g. C₆₋₁₀)) aryl groups. Such groups may be monocyclic, bicyclic or tricyclic and have between 6 and 14 ring carbon atoms, in which at least one ring is aromatic. The point of attachment of aryl groups may be via any atom of the ring system. However, when aryl groups are bicyclic or tricyclic, they are linked to the rest of the molecule via an aromatic ring. C₆₋₁₄ aryl groups include phenyl, naphthyl and the like, such as 1,2,3,4-tetrahydronaphthyl, indanyl, indenyl and fluorenyl. Most preferred aryl groups include phenyl.

The term “heteroaryl” when used herein refers to an aromatic group containing one or more heteroatom(s) (e.g. one to four heteroatoms) preferably selected from N, O and S (so forming, for example, a mono-, bi-, or tricyclic heteroaromatic group). Heteroaryl groups include those which have between 5 and 14 (e.g. 10) members and may be monocyclic, bicyclic or tricyclic, provided that at least one of the rings is aromatic. However, when heteroaryl groups are bicyclic or tricyclic, they are linked to the rest of the molecule via an aromatic ring. Heterocyclic groups that may be mentioned include benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. Particularly preferred heteroaryl groups include pyridyl, pyrrolyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, imidazolyl, pyrimidinyl, indolyl, pyrazinyl, indazolyl, pyrimidinyl, thiophenetyl, thiophenyl, pyranyl, carbazolyl, acridinyl, quinolinyl, benzoimidazolyl, benzthiazolyl, purinyl, cinnolinyl and pterdinyl. Particularly preferred heteroaryl groups include monocylic heteroaryl groups.

By “linear oligo or poly-ethyleneoxy groups” we mean an oligo- or poly-ethyleneoxy chain of the following formula —(CH₂—CH₂—O)ₓₓ—CH₃, wherein xx can be from 2 to 100 (such from about 3 to about 20, e.g. where xx is 3) provided that the total number of ethylene oxy groups does not exceed 100.

By “branched oligo or poly-ethyleneoxy groups” we mean an oligo or poly-ethyleneoxy chain wherein one or more —(CH₂—CH₂—O)— units is replaced by a unit that allows the incorporation of a branch-point in the oligo- or poly-ethyleneoxy unit (e.g. —(CH(—O—CH₂—CH₂—O—)—CH₂—O)—).

For the avoidance of doubt, in cases in which the identity of two or more substituents in a compound of formula I may be the same, the actual identities of the respective substituents are not in any way interdependent.

Compounds of formula I or formula III that may be mentioned include those in which:

b represents a double bond and D represents —CH₂—.

Compounds of formula I that may be mentioned in clude those in which:

b represents a single bond and D represents —C(O)— or —CH₂—.

Compounds of formula I or formula III that may be mentioned include those in which:

b represents a single bond and D represents —C(O)— or —CH₂—, wherein the stereochemistry is as defined in formula IA below,

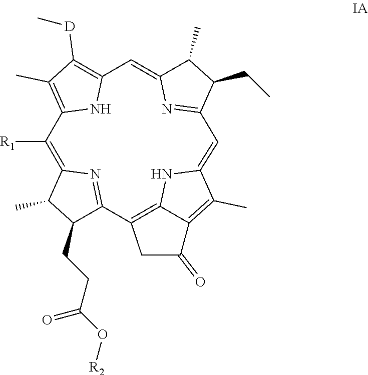

wherein R₁ and R₂ are as hereinbefore defined.

Yet further compounds of formula I or formula III that may be mentioned include those in which:

b represents a single bond and D represents —CH₂—.

Compounds of formula I or formula III that may be mentioned include those in which:

R₁ represents —(CH₂)ᵤ—C(R₃)═C(R₄)—(CH₂)ᵥ—X, or —(CH₂)_{w}—C≡C—(CH₂)ₓ—X;

the sum of w and x is from 2 to 10;

X represents —C(O)-L₁, —OH, —CN, —SH, —NHR₃ₐ, halo, phosphoramidityl, N-hydroxy succinimidyl ester, sulfo-N-hydroxy succinimidyl ester, fluorophenyl esters, isothiocyanato, iodoacetamidyl, maleimidyl, aryl or hetroaryl (which latter two groups are substituted by one or more groups selected from —C(O)-L₁, —OH, a sulfonyl ester (e.g. mesylate, tosylate), —NO₂, —CHO, —N₃, —CN, —SH, —NHR₃ₐ, halo, phosphoramidityl, N-hydroxy succinimidyl ester, sulfo-N-hydroxy succinimidyl ester, fluorophenyl esters, isothiocyanato, iodoacetamidyl and maleimidyl);

L₁ represents —OH or —O—C(O)—R₅, or —C(O)-L₁ represents a carboxylic acid functional group activated by a carbodiimide;

R₂ represents alkyl, cycloalkyl, alkylenyl, alkynyl, aryl, benzyl, heteroaryl (wherein the latter three groups may be substituted by one or more groups selected from —OH, —NH₂ or a C1 to C6 alkyl substituted by one or more halo atoms) independently substituted by one or more linear or branched oligo or poly-ethyleneoxy groups (wherein the total number of oligo or poly-ethyleneoxy groups is from 2 to 100 (e.g. about 3 to about 20)), or R₂ represents —NR₆(R₇) or —N(R₆ₐ)—(CH₂)—SO₃⁻E⁺;

R₆ and R₇ independently represent —(CH₂)_{z}—NR₈(R₉) or —(CH₂)_{z}—N⁺R₈(R₉)(R₁₀)A⁻, provided that at least one of R₆ and R₇ are not H;

R₆ₐ represents H or C1 to C3 alkyl optionally substituted by one or more groups selected from —OH or halo;

z represents 1 to 10;

R₈ to R₁₀ independently represents H, alkyl or alkenyl optionally substituted by one or more groups selected from —OH and halo;

A⁻ represents I⁻, Cl⁻, Br⁻.

Further compounds of formula I or formula III that may be mentioned include those in which:

R₁ represents —(CH₂)_{w}—C≡C—(CH₂)ₓ—X;

the sum of w and x is from 2 to 10;

X represents —C(O)-L₁, phosphoramidityl, N-hydroxy succinimidyl ester, sulfo-N-hydroxy succinimidyl ester, fluorophenyl esters, isothiocyanato, iodoacetamidyl or maleimidyl;

L₁ represents —OH or —C(O)-L₁ represents a carboxylic acid functional group activated by a carbodiimide;

R₂ represents aryl, benzyl, heteroaryl (wherein the latter three groups may be substituted by one or more groups selected from —OH, —NH₂ or a C1 to C6 alkyl substituted by one or more halo atoms) independently substituted by one or more linear or branched oligo or poly-ethyleneoxy groups (wherein the total number of oligo or poly-ethyleneoxy groups is from 2 to 100 (e.g. about 3 to about 20)), or R₂ represents —NR₆(R₇) or —N(R₆ₐ)—(CH₂)—SO₃⁻E⁺;

R₆ and R₇ independently represent —(CH₂)_{z}—NR₈(R₉) or —(CH₂)_{z}—N⁺R₈(R₉)(R₁₀)A⁻, provided that at least one of R₆ and R₇ are not H;

R₆ₐ represents H;

z represents 1 to 10;

R₈ to R₁₀ independently represents H or alkyl optionally substituted by one or more groups selected from —OH or halo;

A⁻ represents I⁻, Cl⁻, Br⁻.

Yet further compounds of formula I or formula III that may be mentioned include those in which:

R₁ represents —(CH₂)_{w}—C≡C—(CH₂)ₓ—X;

the sum of w and x is from 2 to 10;

X represents —C(O)-L₁, phosphoramidityl, N-hydroxy succinimidyl ester, sulfo-N-hydroxy succinimidyl ester, fluorophenyl esters, isothiocyanato, iodoacetamidyl or maleimidyl;

L₁ represents —OH or —C(O)-L₁ represents a carboxylic acid functional group activated by a carbodiimide;

R₂ represents aryl, benzyl, heteroaryl (wherein the latter three groups may be substituted by one or more groups selected from —OH, —NH₂ or a C1 to C6 alkyl substituted by one or more halo atoms) independently substituted by one or more linear or branched oligo or poly-ethyleneoxy groups (wherein the total number of oligo or poly-ethyleneoxy groups is from 2 to 100 (e.g. about 3 to about 20)).

Yet further compounds of formula I or formula III that may be mentioned include those in which:

R₁ represents —(CH₂)_{w}—C≡C—(CH₂)ₓ—X;

the sum of w and x is from 2 to 10;

X represents —C(O)-L₁, phosphoramidityl, N-hydroxy succinimidyl ester, sulfo-N-hydroxy succinimidyl ester, fluorophenyl esters, isothiocyanato, iodoacetamidyl or maleimidyl;

L₁ represents —OH or —C(O)-L₁ represents a carboxylic acid functional group activated by a carbodiimide;

R₂ represents —NR₆(R₇);

R₆ and R₇ independently represent —(CH₂)_{z}—NR₈(R₉) or —(CH₂)_{z}—N⁺R₈(R₉)(R₁₀)A⁻, provided that at least one of R₆ and R₇ are not H;

z represents 1 to 10;

R₈ to R₁₀ independently represents H or alkyl optionally substituted by one or more groups selected from —OH or halo;

A⁻ represents I⁻, Cl⁻, Br⁻.

Compounds of formula I or formula III that may be mentioned include those in which:

R₁ represents a structural fragment of formula Ia, Ib, Ic, Id, Ie, If as hereinbefore defined;

R¹¹ represents H, alkyl (optionally substituted by one or more groups selected from —OH, halo), or linear or branched ethyleneoxy groups (wherein the total number of oligo or poly-ethyleneoxy groups is from 2 to 100 (e.g. about 3 to about 20));

R₁₂ to R₁₄ independently represent H or C1 to C6 alkyl optionally substituted by one or more groups selected from —OH, halo and linear or branched ethyleneoxy groups (wherein the total number of oligo or poly-ethyleneoxy groups is from 2 to 100 (e.g. about 3 to about 20));

Y⁻ represents I⁻, Br⁻ or Cl⁻;

R₂ represents —C(O)-L₃, phosphoramidityl, N-hydroxy succinimidyl ester, sulfo-N-hydroxy succinimidyl ester, fluorophenyl esters (e.g. 1,2,3,5,6-pentafluorophenyl, 4-sulfo-2,3,5,6-pentafluorophenyl), isothiocyanato, iodoacetamidyl or maleimidyl;

L₃ represents —OH or —O—C(O)—R₁₅, halo, an activated ester such as 1-oxybenzotriazoyl or an aryloxy group optionally substituted with one or more subsistent selected from nitro, fluoro, chloro, cyano and trifluoromethyl, or —C(O)-L₁ represents a carboxylic acid functional group activated by a carbodiimide;

R₁₅ represents C1 to C6 alkyl optionally substituted by one or more groups selected from —OH and halo.

Compounds of formula I or formula III that may be mentioned include those in which:

R₁ represents a structural fragment of formula Ia, Ib, Ic, Id, Ie, If as hereinbefore defined;

R¹¹ represents alkyl, or linear or branched ethyleneoxy groups (wherein the total number of oligo or poly-ethyleneoxy groups is from about 3 to about 20);

R₁₂ to R₁₄ independently represent H or C1 to C6 alkyl optionally substituted by one or more groups selected from —OH, halo and linear or branched ethyleneoxy groups (wherein the total number of oligo or poly-ethyleneoxy groups is from about 3 to about 20);

Y⁻ represents I⁻, Br⁻ or Cl⁻;

R₂ represents —C(O)-L₃, phosphoramidityl, N-hydroxy succinimidyl ester, sulfo-N-hydroxy succinimidyl ester, fluorophenyl esters (e.g. 1,2,3,5,6-pentafluorophenyl, 4-sulfo-2,3,5,6-pentafluorophenyl), isothiocyanato, iodoacetamidyl or maleimidyl;

L₃ represents —OH or —O—C(O)—R₁₅, or —C(O)-L₁ represents a carboxylic acid functional group activated by a carbodiimide;

R₁₅ represents C1 to C6 alkyl optionally substituted by one or more groups selected from —OH.

In an alternative embodiment, there is provided a compound of Formula II:

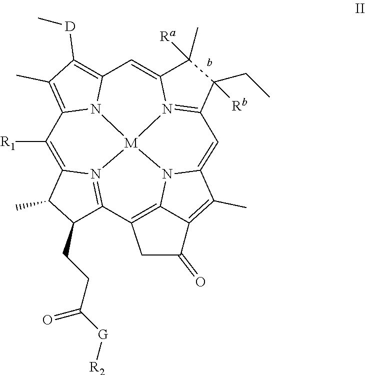

wherein D, b, R₁, R₂, G, R² and R^{b} are as hereinbefore defined; and

M represents Zn(II), Fe(II), Ga(II), Co(II), Cu(II), Mn(II), Ni(II), Ru(II), AI(II), Pt(II) or Pd(II).

Particularly preferred compounds according to the invention are provided below as compounds of formula IB to IG:

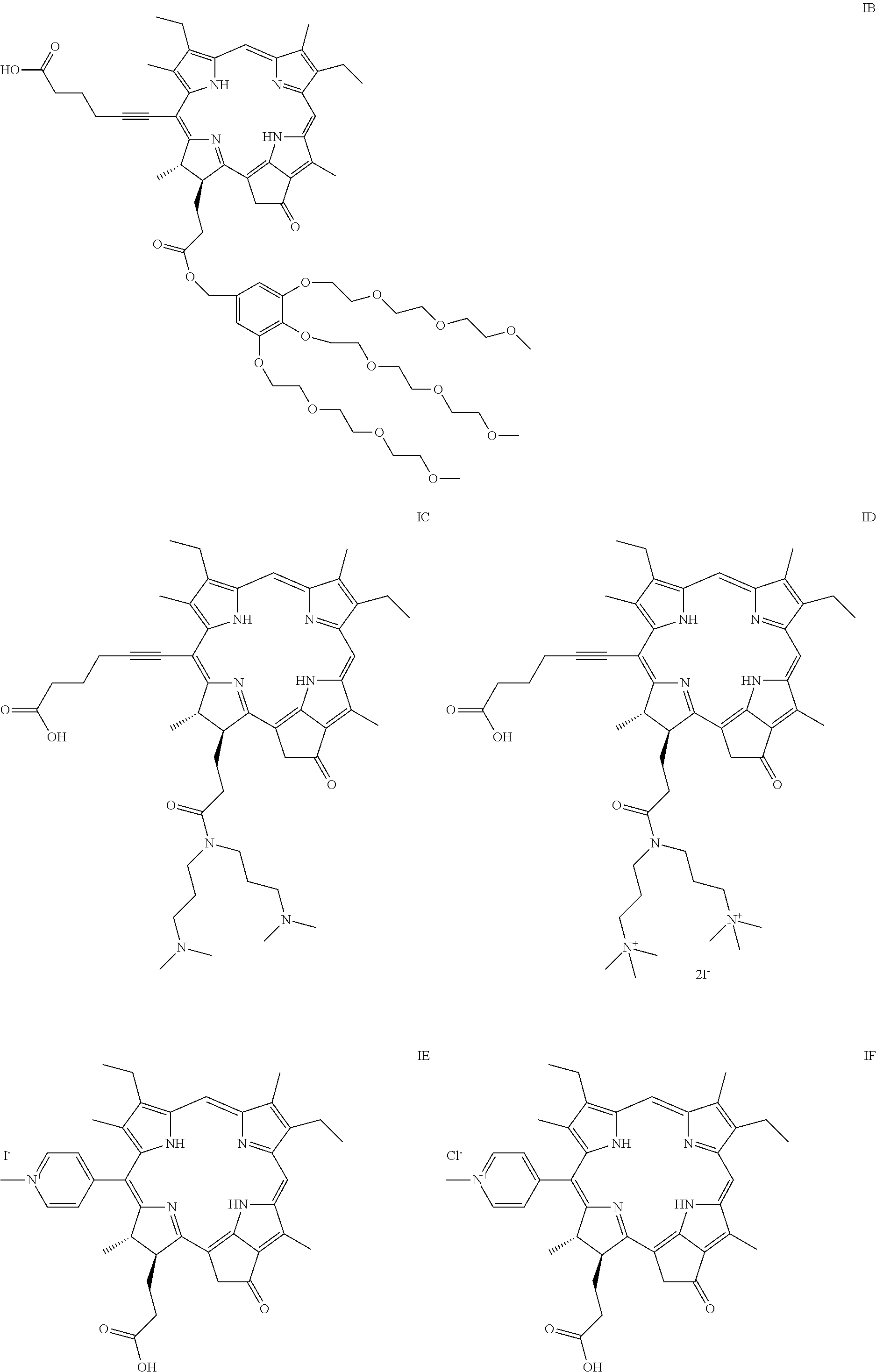

By “reactive with a single amine or thiol” it is intended that the functional group is suitable for reacting with an amine or thiol group on an amino acid comprised in the peptide carrier, or on another type of carrier with available amine or thiol groups. Examples of amino acids that have amine or thiol groups available for conjugation are lysine, arginine, histidine and cysteine.

It is intended that any compound of Formulas I to III may be suitable for conjugation to proteins through appropriate activation of the conjugation handle. When the conjugation handle is a functional group terminating in a carboxylic acid group the activation may be by converting the group into an activated succinimidyl ester. This may be achieved, for example, with N-hydroxy succinimide and DCC, as explained in Example 1.

It is intended that any of the disclosed photosensitising compounds may be coupled to an antibody, or a fragment or derivative thereof. The compounds that are suitable for such conjugation are clearly disclosed herein.

The invention further encompasses any novel intermediate chemical compound as disclosed in Example 1.

In a further aspect the present invention provides a process of making a compound comprising a photosensitising agent, which comprises a compound of any one of Formulas I to III, coupled to a carrier molecule comprising the steps of:

(i) providing a photosensitising agent comprising a compound of any one of Formulas I to III;

(ii) providing a carrier molecule;

(iii) conjugating the photosensitizing agent and the carrier molecule in the presence of a first and a second polar aprotic solvent and an aqueous buffer.

It is envisaged that the photosensitising agent is any compound that falls within the definition of any one of Formulas I to III provided in the present application. A preferred embodiment of the photosensitising agent includes the compound of any one of Formulas I to III, wherein R₁ is hexynoic acid and R₂ is a benzy ether unit with short tri(ethylene glycol) monomethyl ether (TEG) chains (compound (10) of scheme 2, in Example 1, which is converted to compound (II) before conjugation).

Preferably, the compound comprises a ratio of photosensitising agent to carrier molecule of at least 3:1. Preferably the ratio of photosensitising agent to carrier molecule is more than 5:1 or more preferably more than 10:1. The ratio may be between 5:1 and 10:1 or higher. For example, the ratio may be 20:1 or 40:1 or higher. The ratio may be between 10:1 and 20:1 or between 20:1 or 40:1. It is envisaged that when the carrier is an svFv then the ratio may be up to 20:1 or higher. It is further envisaged that then the carrier is an Fab or diabody then the ratio may be up to 40:1 or higher. A ratio of 40:1 may be expected to equate to a substitution of around 10% of the total protein.

Preferably, the functional and physical properties of the photosensitising agent and the carrier molecule are substantially unaltered after coupling.

Appropriate polar aprotic solvents from which the first and second polar aprotic solvent are selected, but are not limited to, include the group comprising: dimethyl sulfoxide (DMSO); acetonitrile; N,N-dimethylformamide (DMF); HMPA; dioxane; tetrahydrofuran (THF); carbon disulfide; glyme and diglyme; 2-butanone (MEK); sulpholane; nitromethane; N-methylpyrrolidone; pyridine; and acetone. Other polar aprotic solvents which may be used are well known to those skilled in the art. The total amount of both polar aprotic solvents relative to the aqueous mixture should be about 50% by volume.

The relative amounts of the 2 polar aprotic solvents to each other can vary from 1 to 49%:49% to 1.

Preferably, the first and second aprotic solvent are selected from the group consisting of: DMSO; DMF; and acetonitrile. More preferably, the first and second aprotic solvent are DMSO and acetonitrile.

Even more preferably, the ratio of aqueous buffer to first aprotic solvent to second aprotic solvent is approximately 50%:1 to 49%:49 to 1%.

Even more preferably, the aprotic solvent mixture is 92% PBS: 2% DMSO: 6% acetonitrile and the step of conjugating the photosensitizing agent and the carrier molecule is conducted at a temperature of between 0° C. and 5° C. Alternatively, the conjugation step may be conducted at room temperature or higher. By “room temperature” or “RT” is meant a temperature of about 10° C. to about 30° C., more preferably this may be a temperature of above 15° C. to about 25° C. The combination of solvents keeps the whole reaction homogeneous and by carrying out the coupling for approximately only 30 min, we are able to achieve high coupling ratios and very low degrees of non-covalent binding. It is envisaged that carrying out the coupling at lower temperatures to stabilise the protein but that higher temperatures may provide higher coupling ratios.

The invention further provides a process wherein the carrier molecule is an antibody fragment and/or a derivative thereof. Preferably, the antibody fragment and/or derivative is a single-chain antibody, and may conveniently be an scFv. The carrier molecule is preferably humanised or human.

Using the above protocol, photosensitisers with carboxylic acid groups derivatised to form active esters may be coupled efficiently and with high molar ratio to antibody fragments via surface-accessible lysine residues. Pyropheophorbide a (PPA) is a photosensitiser derived from natural products, and apart from excellent photophysics which makes it an ideal photosensitiser, it possesses a single propionic acid side chain. The PPA propionic acid function may be readily converted to the corresponding N-hydroxysuccinimide ester (NHS) or ‘active ester’ and purified through a combination of chromatography and recrystallisation to obtain very pure derivatives ready for conjugation, and thereafter coupled efficiently to antibody fragments.

The compounds of Formulas I to III are derived from PPA, as described in Example 1. The conversion of the preferred derivatives into the form appropriate for conjugation is also described in Example 1. The photosensitising agent may be described as a monofunctional photosensitiser.

Many photosensitising agents described in the art have multiple reactive functionalities. The presence of multiple reactive functionalities on the photosensitiser can lead to a number of problems. It is difficult to obtain sufficiently pure material to control the stoichiometry of the conjugation reaction and as a consequence reactions are carried out using large excesses of the reactive photosensitiser resulting in increased non-covalent binding. Intramolecular antibody cross-linking can also occur during conjugation resulting in low coupling yields and increased aggregate formation.

Our work with antibody fragments has shown that by controlling the stoichiometry of the photosensitiser during the conjugation and having lysine residues sufficiently spaced apart geometrically can lead to photoimmunoconjugates with high photosensitiser loadings and excellent PDT activity.

It is preferred that the process of conjugation of the photosensitiser of the invention to the carrier molecule is carried out at a concentration of carrier molecule of 250 pg/ml or higher. This may be a concentration of between 250 pg/ml and 5 mg/ml. Alternatively, it could be a concentration of more than 1 mg/ml, such as 2, 3, 4 or 5 mg/ml. Preferably the concentration of carrier molecule is about 5 mg/ml or higher. For example the concentration of carrier molecule may be up to 10 mg/ml or higher. Such concentrations are particularly contemplated when the carrier molecule is a peptide. More preferably, these concentrations of carrier are applicable when the carrier is an antibody or fragment thereof. The ability to perform the conjugation steps at higher carrier concentrations (e.g. higher antibody concentrations) stems from the higher solubility of the compounds of Formulas I to III in aqueous solutions than photosensitisers provided in the art. Performing the conjugation step at higher antibody concentrations will lead to higher concentration photoimmunoconjugates. It is envisaged that this will have beneficial effects on the overall outcome of the therapy cycle as a higher dose of agent can be administered to the patient.

Conveniently, the process of the present invention may further comprises the following step performed after step (iii):

(iv) coupling a modulating agent to the carrier molecule, wherein the modulating agent is capable of modulating the function of the photosensitising agent.

As well as coupling photosensitisers to ligands, it is also possible, using similar coupling chemistries to couple other molecules to the ligands in such a way that they modify the photophysical or photodynamic properties of the overall photo-immunoconjugate. These alternative molecules can be coupled to the same residue type as the photosensitisers (i.e. before or after photosensitiser coupling) at stoichiometric ratios to allow both types of molecules to be coupled/accommodated on different residue types (e.g. photosensitiser coupled onto lysines and subsequently modifying chemical coupled to aspartate/glutamate residues).

Photodynamic modulators may serve to alter the types and amounts of reactive oxygen species generated upon light illumination of the photosensitiser. For example photosensitisers which generate a more type II reaction (i.e. singlet oxygen) can be modulated to generate more type I reaction with high concentrations of superoxide and hydroxide radicals. This could have major implications on the PDT potency or therapeutic outcome. For example a photo-immunoconjugate targeting a non-internalising tumour antigen may be more potent if it generated a predominantly type I reaction at the surface of the cell, causing membrane damage and being less susceptible to anti-oxidant responses such as superoxide dismutase (which is generated intracellularly).

Preferably, the modulating agent is selected from the group consisting of: benzoic acid; benzoic acid derivatives containing an azide group like 4-azidotetrafluorophenylbenzoic acid and other aromatic or heteroaromatic groups containing an azide moiety (N₃) including polyfluorobenzenes, naphthalines, napthaquinones, anthracenes, anthraquinones, phenanthrenes, tetracenes, naphthacenediones, pyridines, quinolines, isoquinolines, indoles, isoindoles, pyrroles, imidazoles, pyrazoles, pyrazines, benzimidazoles, benzofurans, dibenzofurans, carbazoles, acridiens acridones, and phenanthridines, xanthines, xanthones, flavones and coumarins. Aromatic and heteroaromatic sulfenates derived from the aromatic/heteroaromatic groups above. Other specific modulating agents include vitamin E analogues like Trolox, butyl hydroxyl toluene, propyl gallate, deoxycholic acid and ursadeoxycholic acid. One example of a chemical modifier which can be coupled to a ligand alongside the photosensitising agent is the succinimidyl ester of benzoic acid (BA).

This has been shown to result in more potent PDT cell killing in vitro when co-coupled with PPa to an anti-CEA scFv compared to the scFv coupled with PPa alone.

Preferably, the process further comprises the following step performed after step (iii) or (iv):

(v) combining the compound with a pharmaceutically-acceptable carrier to form a pharmaceutical formulation.

The process of the invention may also include the optional step of coupling a visualising agent to the conjugate. Alternatively the photosensitising agent forming part of the conjugate may also be used as a visualising agent.

The visualising agent may be a fluorescent or luminescent dye. Alternatively, or additionally, the visualising agent may be an MRI contrast agent. By “MRI contrast agent” is meant contrast agents for Magnetic Resonance Imaging, as would be well understood in the art. Many MRI contrast agents that are approved for use in medicine are Gadoliniom-based agents. Appropriate agents for use in the context of the present invention may include non-ionic agents, iodinated contrast materials, ionic chelates, ultrasmall supermagnetic oxide particles and any suitable agent that would be known to a person of skill in the art. For example, the MRI contrast agent may be Gadodiamide or Gadoteridol.

The use of recombinant antibodies in immuno-assays or diagnostics is a well studied area. The exquisite specificity, high affinity and versatility of antibodies and antibody fragments make them ideal binding molecules as part of a detection system. For example, in medical imaging, antibodies have been linked to optically-active compounds such as fluorescent dyes and used to detect pre-cancerous and cancerous lesions, measuring treatment response and early detection of recurrences [95] and in vitro, transmissible spongiform encephalopathies (prion diseases) have been detected with fluorescently labelled antibodies [96].

Clinically useful tumour imaging requires detection of small lesions. The benefits of detection can then be realised by early action. One of the problems associated with conventional imaging techniques is poor tumour to background contrast. Various strategies have been developed to increase the localization of targeting molecules in tumours and to reduce their uptake by normal tissue, thus improving tumour:tissue ratio. These approaches include developing small tumour specific peptide molecules with favourable pharmacokinetics [97], improved labelling techniques [98], using pre-targeting strategies, modifying tumour delivery and up-regulating of tumour marker expression. In addition, several new dyes have been developed [99]. Far-red fluorochromes have been synthesized that have many properties desirable for in vivo imaging. Far-red fluorochromes absorb and emit at wavelengths at which blood and tissue are relatively transparent, have high quantum yields, and have good solubility even at higher molar ratios of fluorochrome to antibody. Small antibody species such as single-chain Fv fragments possess pharmacokinetics which can result in good contrast ratios, but clear rapidly resulting in low absolute levels of reporter groups in the target tissue. Higher fluorescent yields can compensate for this lower deposition increasing the sensitivity of detection.

Other applications of imaging include the development of research tools. Antibodies labelled with dyes have been invaluable in visualising cell biological processes such as receptor trafficking [100]. Increased fluorescent yields would enable the detection and monitoring of low abundance molecules. The usual method for visualising labelled cells is immunofluorescent microscopy where multiply-labelled molecules can be simultaneously monitored using a range of specific antibodies possessing different and non-overlapping fluorescence emission spectra.

The coupling of dye molecules to antibody fragments or other appropriate ligands, using the disclosed coupling conditions, results in higher loading ratios. This can translate directly into enhanced photophysics. As well as higher singlet oxygen generation for improved PDT, superior photophysics can manifest as increased fluorescence. Antibody fragment photo-immunoconjugates with appropriate dye molecules can make more effective diagnostic reagents due to their favourable pharmacokinetics and enhanced fluorescence. Rapid clearance and low non-specific tissue binding will lead to very high contrast ratios and high fluorescence will allow more sensitive detection of the output signal. The use of antibody fragments, constructed, selected or engineered to contain favourably-spaced functional groups for coupling (e.g. lysine amino groups) as described above can lead to dyes with more favourable fluorescence yields due to reduced quenching and mis-interactions. This will have applications primarily in medical imaging, but can also be used to make more sensitive reagents for diagnostic kits or cellular imaging and by coupling fluorescent dyes and photosensitisers to the same antibody fragments a bifunctional agent can be produced, allowing both tumour imaging and phototherapy.

In a further aspect of the invention there is provided a compound obtainable by the process of the invention. The compound may be expected to comprise a carrier coupled to a photosensitiser of the invention. In an emobidiment of this aspect, the invention contemplates that the compound obtainable by the process of the invention would comprise a photosensitiser of the invention coupled to a carrier molecule with a minimum coupling ratio of 3:1. The coupling ratio may be 5:1, 10:1, 20:1, 40:1 or any value in between these values, or alternatively the coupling ratio may be higher. It is envisaged that in a further embodiment, the carrier molecule would be able to bind selectively to a target cell.

Preferably the carrier molecule has an upper size limit of 3:1 when compared to the photosensitiser, typically an upper limit of 30 kDa. An example of such a carrier is an scFv.

Advantageously the functional and physical properties of the photosensitising agent and the carrier molecule are substantially unaltered in the coupled form in comparison to the properties when in an uncoupled form.

Preferably, the carrier molecule is selected from the group consisting of: an antibody fragment and/or a derivative thereof, or a non-immunogenic peptide ligand.

Conveniently the antibody fragment and/or derivative thereof is a single-chain antibody fragment, in particular an scFv.

Alternatively the carrier molecule is humanised or human.

Preferably, the photosensitising agent is a compound of Formula I as described in the present application.

Conveniently, the photosensitising agent is coupled to the carrier molecule at an amino acid residue or a sugar molecule on the carrier molecule.

Preferably the amino acid residue is at least one selected from the group consisting of: lysine; cysteine; tyrosine; serine; glutamate; aspartate; and arginine. Alternatively, the sugar molecule is selected from at least one of the group consisting of: sugars comprising an hydroxyl group; sugars comprising an aldehyde group; sugars comprising an amino group; and sugars comprising a carboxylic acid group.

Although coupling photosensitisers to lysine residues is generally straightforward, the above conjugation methodology can also apply to the coupling of photosensitisers to antibody fragments via other amino acid residues or sugar molecules attached to the protein by N- or O-linked glycosylation using different functional groups on the photosensitiser moieties. Table 1 lists these residues and the other possible coupling chemistries which can be used with this coupling method.

TABLE 1
Functional groups for coupling photosensitizers onto antibodies

| Residue(s) | Functional group | Coupling chemistry | Resulting bond |
|---|---|---|---|
| Lysine | Amine | Active-ester | Amide |
| | | Isothiocyanate | Isothiourea |
| | | Isocyanates | Isourea |
| | | Acyl azides | Amide |
| | | Sulphonyl chloride | Sulphonamide |
| | | Carbonyl, reduce. | Schiff Base, 2° amine |
| | | Epoxide | 2° Amine |
| | | Carbonates | Carbamate |
| | | Fluorobenzene deriv. | Arylamine |
| | | Imidoesters | Amidine |
| | | Carbodiimides | Amide |
| | | Anhydrides | Amide |
| Cysteine | Thiol | Haloacetyl | Thioether |
| | | Maleimides | Thioether |
| | | Acryloyl | Thioether |
| | | Activated aryl deriv. | Arylthioether |
| | | Active-ester | Thioester |
| | | Carbodiimide | Thioester |
| | | Redox reactions | Disulphide |
| Tyrosine, | Hydroxyl | Diazonium | Diazo |
| serine | | Mannich | 2°amine |
| | | Active-ester | Ester |
| | | Active Alkylation | Ether |
| | | Isocyanates | Carbamate |
| Glutamate, | Carboxylic | Diazoalkyl | Ester |
| aspartate | acid | Carbodiimides | Amide, Ester, Thioester |
| | | Acylimidazole | Amide, Ester, Thioester |
| Arginine | Guanidinyl | Dicarbonyl | Schiff base |
| Sugars | Hydroxyl (e.g. | Acylation | Ester |
| | glucose) | Alkylation | Ether |
| | | Oxidative cleavage to | Schiff base, mild redn. |
| | | the aldehyde | to the 2° amine |
| Sugars | Aldehyde (e.g. | Reductive amination | Schiff base, 2° amine |
| | mannose) | | |
| Sugars | Amino (e.g. b-D- | See reactions for | See reactions for |
| | mannosamine) | lysine | lysine |
| Sugars | Carboxylic acid | See reactions for | See reactions for |
| | (e.g. sialic acid) | glutamate | glutamate |

Antibody fragments vary in amino acid sequence and the number and spacing of functional groups to couple photosensitizers to. The most common frequently used functional group for conjugation is the primary amine found at the N-terminus and on lysine residues, as described above. We have found, surprisingly, that a major determinant of the effectiveness of a particular photosensitiser-antibody fragment conjugate is the spatial separation of the residues to which photosensitiser molecules are attached. These residues must be distinct and topologically separated on the surface of the antibody for effective coupling and optimal photophysics of the resulting conjugate.

A more detailed analysis of the variable regions of human immunoglobulins, in the context of the optimal positions where photosensitisers may be coupled, is provided in WO 2007/042775.

It is envisaged that the photosensitising agents are spaced apart on the carrier molecule so as to minimise interactions. Therefore, the residues upon which the photosensitising agents are coupled should not be too close to one another. A definition of a residue being close to another can be one that is adjacent in the 3-dimensional structure.

Alternatively, a residue may be separated according to the primary sequence, but adjacent in space due to the structure of the fold of the antibody domain. A directly adjacent residue can be defined as 3-4 angstroms apart in space.

We have found that the coupling of photosensitisers onto lysine residues which are directly adjacent will result in photophysical quenching and poorer photodynamic effects (such as increased aggregation and poorer solubility of photo-immuno conjugates). Coupling is more effective when lysine residues are further separated, preferably two amino acids apart (3.5 to 7.5 angstroms), more preferably three amino acids apart (9 to 12 angstroms), more preferably four amino acids apart (10-15 nm), even more preferably five amino acids apart (15-20 nm), yet even more preferably six amino acids apart (20-25 nm). Antibodies should be chosen, selected or engineered to possess these properties. The more lysine residues an antibody possess, with more optimal separation, the better that antibody will be at forming effective and potent photo-immuno conjugates with optimal photophysical and photodynamic effects.

Methods of determining whether amino acid residues for photosensitiser coupling are close or adjacent to one another are well known in the art. Clustal sequence alignment (using web resources such as http://www.ebi.ac.uk/clustalw/European bioinformatics Institute) is a well established tool for comparing primary amino-acid sequence. Furthermore, in the absence of full 3-dimensional structural data for an antibody fragment, it is possible to use well-established techniques such as homology modelling using known structures (for example that of a murine scFv [89] to deduce probable structure of the antibody fragment, and thereby to identify whether residues for coupling are close or adjacent in space. The high degree of homology exhibited by antibodies and antibody fragments means these techniques can be applied with a high degree of confidence. Web resources for homology modelling are available, such as the Expert Bioinformatics Analysis System from the Swiss Institute of Bioinformatics (http://us.expasy.org) which also provides the free desktop modelling programme SwissPDB Viewer (Guex, N. and Peitsch, M.C. (1997) SWISS-MODEL and the Swiss-PdbViewer: An environment for comparative protein modeling. Electrophoresis 18, 2714-2723).

An example of such a favourable distribution of lysine residues on a scFv is provided in WO 2007/042775. If the distribution of lysine residues is less favourable for conjugation and optimal photophysics, the antibody fragment may be altered using standard molecular biological techniques, such as site directed mutagenesis to remove poorly spaced (too closely positioned) or introduce well-spaced residues.

The above concept can also apply to the spacing and coupling to other amino acid residues other than lysine or to sugar molecules attached to the protein by N- or O-linked glycosylation. Table 1 lists these residues and the possible coupling chemistries which can be used.

The above concept can also be applied to non-antibody based ligands. Examples of ligands which can be used to target photosensitisers which can also be influenced by amino acid spacing are listed in Table 2.

TABLE 2
List of antibody and non-antibody based ligands which
could be used in targeted photodynamic therapy

| Type | Ligand name | Reference |
|---|---|---|
| Immunoglobin- | Domain antibodies | 30 |
| based | Single chain Fvs | 70 |
| | Fab fragment | 90 |
| | Fn3 domains | 29 |
| | Protein L | 91 |
| | T cell receptors | 92 |
| Non-immunoglobin | Peptides | 88 |
| | Ankyrin repeats | 32 |
| | Anticalin | 31 |

This will lead to coupled photosensitizers retaining their photophysical properties and therefore good photodynamic therapy function. There are many examples of antibodies where many of the lysine residues are adjacent in primary sequence or in 3-dimensional space. By molecular modelling and site-directed mutagenesis, we are able to engineer the position of these lysine residues, adding additional ones if there are too few, removing adjacent residues or increasing the distance between others.

This leads to antibody fragments which are more amenable to photosensitizer coupling, capable of achieving higher loading (increased photosensitizer: antibody ratios) and more potent PDT effects. One indirect measurement of enhanced photophysics is increased fluorescence.

Advantageously, the compound further comprises a modulating agent wherein the modulating agent capable of modulating the function of the photosensitising agent coupled to the carrier molecule. Preferably the modulating agent is selected from the group of benzoic acid, benzoic acid derivatives containing an azide group like 4-azidotetrafluorophenylbenzoic acid and other aromatic or heteroaromatic groups containing an azide moiety (N₃) including polyfluorobenzenes, naphthalines, napthaquinones, anthracenes, anthraquinones, phenanthrenes, tetracenes, naphthacenediones, pyridines, quinolines, isoquinolines, indoles, isoindoles, pyrroles, imidazoles, pyrazoles, pyrazines, benzimidazoles, benzofurans, dibenzofurans, carbazoles, acridiens acridones, and phenanthridines, xanthines, xanthones, flavones and coumarins. Aromatic and heteroaromatic sulfenates derived from the aromatic/heteroaromatic groups above. Other specific modulating agents include vitamin E analogues like Trolox, butyl hydroxyl toluene, propyl gallate, deoxycholic acid and ursadeoxycholic acid.

Conveniently, the compound further comprises a visualising agent, for example a fluorescent or luminescent dyes (see above). Alternatively, or additionally, the visualising agent may be an MRI contrast agent

A preferred example of the conjugates of the invention is wherein the carrier molecule is a C6 (anti Her-2) scFv and the photosensitising agent is a compound of Formula I, wherein R₁ is hexynoic acid and R₂ is a benzy ether unit with short tri(ethylene glycol) monomethyl ether (TEG) chains (compound (10) of scheme 2, in Example 1, which is converted to compound (II) before conjugation). A compound according to this preferred example demonstrated excellent in vitro cell kills, and was able to differentiate between targeted and non-targeted cells and had minimal dark toxicity. The compound also displayed improved pharmacokinetics resulting in rapid tumour uptake and higher tumour:blood ratios compared to the C6-PPa photoimmunoconjugate, which could be therapeutically very attractive with very little danger of skin photosensitivity. Overall, a compound of the preferred embodiment demonstrated very effective killing of tumour cells in vivo with complete tumour regression being observed after 2 dose/2 light treatments in tumour bearing mice.

In a further aspect of the invention there is provided a use of the compound of the invention in the diagnosis and/or treatment and/or prevention of a disease requiring the destruction of a target cell.

There is also provided the use of the compound of the invention in the manufacture of a medicament for the diagnosis and/or treatment and/or prevention of a disease requiring the destruction of a target cell.

The present invention further provides a compound of the invention for use in the diagnosis and/or treatment and/or prevention of a disease requiring the destruction of a target cell.

Preferably, the disease to be treated is selected from the group consisting of: cancer; age-related macular degeneration; immune disorders; cardiovascular disease; and microbial infections including viral, bacterial or fungal infections, prion diseases, and oral/dental diseases. Examples of prion diseases include Bovine Spongiform Encephalopathy (BSE), Scrapie, Kuru, Creutzfeldt Jakob Disease (CJD) and other transmissible spongiform encephalopathies. Examples of oral/dental diseases include Gingivitis.

Most preferably the disease to be treated is cancer of the colon, lung, breast, Head and neck, brain, tongue, mouth, prostate, testicles, skin, stomach/gastrointestinal, bladder and pre-cancerous lesions such as Barretts oesophagus.

Conveniently the diagnosis of diseases is conducted by visualisation of either the photosensitising agent or an optional visualisation agent such as a fluorescent or luminescent dye.

Advantageously the compound or composition is administered to a patient prior to light exposure.

In a yet further aspect of the invention there is provided a composition comprising the compound of the invention and a pharmaceutically acceptable carrier, excipient or diluent

A further aspect of the invention provides a pharmaceutical formulation comprising a compound according the present invention in admixture with a pharmaceutically or veterinarily acceptable adjuvant, diluent or carrier.

Preferably, the formulation is a unit dosage containing a daily dose or unit, daily sub-dose or an appropriate fraction thereof, of the active ingredient.

The compounds of the invention will normally be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the compounds of the invention can be administered alone but will generally be administered in admixture with a suitable pharmaceutical excipient diluent or carrier selected with regard to the intended route of administration and standard pharmaceutical practice.

For example, the compounds of the invention can be administered orally, buccally or sublingually in the form of tablets, capsules, ovules, elixirs, solutions or suspensions, which may contain flavouring or colouring agents, for immediate-, delayed- or controlled-release applications. The compounds of invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The compounds of the invention can also be administered parenterally, for example, intravenously, intra-arterially, intraperitoneally, intrathecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the compounds of the invention will usually be from 1 mg/kg to 30 mg/kg. Thus, for example, the tablets or capsules of the compound of the invention may contain a dose of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The compounds of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” delivers an appropriate dose of a compound of the invention for delivery to the patient. It will be appreciated that he overall daily dose with an aerosol will vary from patient to patient, and may be administered in a single dose or, more usually, in divided doses throughout the day.

Alternatively, the compounds of the invention can be administered in the form of a suppository or pessary, or they may be applied topically in the form of a lotion, solution, cream, ointment or dusting powder. The compounds of the invention may also be transdermally administered, for example, by the use of a skin patch. They may also be administered by the ocular route, particularly for treating diseases of the eye.

For ophthalmic use, the compounds of the invention can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Generally, in humans, oral or topical administration of the compounds of the invention is the preferred route, being the most convenient. In circumstances where the recipient suffers from a swallowing disorder or from impairment of drug absorption after oral administration, the drug may be administered parenterally, e.g. sublingually or buccally.

For veterinary use, a compound of the invention is administered as a suitably acceptable formulation in accordance with normal veterinary practice and the veterinary surgeon will determine the dosing regimen and route of administration which will be most appropriate for a particular animal.

### MEANINGS OF TERMS USED

The term “antibody fragment” shall be taken to refer to any one of an antibody, an antibody fragment, or antibody derivative. It is intended to embrace wildtype antibodies (i.e. a molecule comprising four polypeptide chains), synthetic antibodies, recombinant antibodies or antibody hybrids, such as, but not limited to, a single-chain modified antibody molecule produced by phage-display of immunoglobulin light and/or heavy chain variable and/or constant regions, or other immunointeractive molecule capable of binding to an antigen in an immunoassay format that is known to those skilled in the art.

The term “antibody derivative” refers to any modified antibody molecule that is capable of binding to an antigen in an immunoassay format that is known to those skilled in the art, such as a fragment of an antibody (e.g. Fab or Fv fragment), or a modified antibody molecule that is modified by the addition of one or more amino acids or other molecules to facilitate coupling the antibodies to another peptide or polypeptide, to a large carrier protein or to a solid support (e.g. the amino acids tyrosine, lysine, glutamic acid, aspartic acid, cysteine and derivatives thereof, NH₂-acetyl groups or COOH-terminal amido groups, amongst others).

The term “scFv molecule” refers to any molecules wherein the V_{H} and V_{L} partner domains are linked via a flexible oligopeptide.

The terms “nucleotide sequence” or “nucleic acid” or “polynucleotide” or “oligonucleotide” are used interchangeably and refer to a heteropolymer of nucleotides or the sequence of these nucleotides. These phrases also refer to DNA or RNA of genomic or synthetic origin which may be single-stranded or double-stranded and may represent the sense or the antisense strand, to peptide nucleic acid (PNA) or to any DNA-like or RNA-like material. In the sequences herein A is adenine, C is cytosine, T is thymine, G is guanine and N is A, C, G or T (U). It is contemplated that where the polynucleotide is RNA, the T (thymine) in the sequences provided herein is substituted with U (uracil). Generally, nucleic acid segments provided by this invention may be assembled from fragments of the genome and short oligonucleotide linkers, or from a series of oligonucleotides, or from individual nucleotides, to provide a synthetic nucleic acid which is capable of being expressed in a recombinant transcriptional unit comprising regulatory elements derived from a microbial or viral operon, or a eukaryotic gene.

The terms “polypeptide” or “peptide” or “amino acid sequence” refer to an oligopeptide, peptide, polypeptide or protein sequence or fragment thereof and to naturally occurring or synthetic molecules. A polypeptide “fragment,” “portion,” or “segment” is a stretch of amino acid residues of at least about 5 amino acids, preferably at least about 7 amino acids, more preferably at least about 9 amino acids and most preferably at least about 17 or more amino acids. To be active, any polypeptide must have sufficient length to display biological and/or immunological activity.

The terms “purified” or “substantially purified” as used herein denotes that the indicated nucleic acid or polypeptide is present in the substantial absence of other biological macromolecules, e.g., polynucleotides, proteins, and the like. In one embodiment, the polynucleotide or polypeptide is purified such that it constitutes at least 95% by weight, more preferably at least 99% by weight, of the indicated biological macromolecules present (but water, buffers, and other small molecules, especially molecules having a molecular weight of less than 1000 daltons, can be present).

The term “isolated” as used herein refers to a nucleic acid or polypeptide separated from at least one other component (e.g., nucleic acid or polypeptide) present with the nucleic acid or polypeptide in its natural source. In one embodiment, the nucleic acid or polypeptide is found in the presence of (if anything) only a solvent, buffer, ion, or other component normally present in a solution of the same. The terms “isolated” and “purified” do not encompass nucleic acids or polypeptides present in their natural source.

The term “recombinant,” when used herein to refer to a polypeptide or protein, means that a polypeptide or protein is derived from recombinant (e.g., microbial, insect, or mammalian) expression systems. “Microbial” refers to recombinant polypeptides or proteins made in bacterial or fungal (e.g., yeast) expression systems. As a product, “recombinant microbial” defines a polypeptide or protein essentially free of native endogenous substances and unaccompanied by associated native glycosylation. Polypeptides or proteins expressed in most bacterial cultures, e.g., E. coli, will be free of glycosylation modifications; polypeptides or proteins expressed in yeast will have a glycosylation pattern in general different from those expressed in mammalian cells.

The term “expression vector” refers to a plasmid or phage or virus or vector, for expressing a polypeptide from a DNA (RNA) sequence. An expression vehicle can comprise a transcriptional unit comprising an assembly of (1) a genetic element or elements having a regulatory role in gene expression, for example, promoters and often enhancers, (2) a structural or coding sequence which is transcribed into mRNA and translated into protein, and (3) appropriate transcription and translation initiation and termination sequences. Structural units intended for use in yeast or eukaryotic expression systems preferably include a leader sequence enabling extracellular secretion of translated protein by a host cell. Alternatively, where recombinant protein is expressed without a leader or transport sequence, it may include an amino terminal methionine residue. This residue may or may not be subsequently cleaved from the expressed recombinant protein to provide a final product.

The terms “selective binding” and “binding selectivity” indicates that the variable regions of the antibodies of the invention recognise and bind polypeptides of the invention exclusively (i.e., able to distinguish the polypeptide of the invention from other similar polypeptides despite sequence identity, homology, or similarity found in the family of polypeptides), but may also interact with other proteins (for example, S. aureus protein A or other antibodies in ELISA techniques) through interactions with sequences outside the variable region of the antibodies, and in particular, in the constant region of the molecule. Screening assays to determine binding selectivity of an antibody of the invention are well known and routinely practiced in the art.

The term “binding affinity” includes the measure of the strength of binding between an antibody molecule and an antigen.

The term “coupling ratio” means the number of molecules of photosensitising agent coupled to one carrier molecule.

The term “carrier molecule” includes the meaning of any agent to which the photosensitising agent is coupled. In particular, the carrier molecule may be a small compound including but not limited to antibody fragments and non-immunogenic peptides.

The term “monofunctional photosensitiser” or “monofunctional phosensitising agent” means-a photosenstiser like PPa which contains a single propionic acid side chain which can be activated and coupled or by the use of chemistry known in the art a senstiser can be modified through protection/deprotection chemistry to possess a group that can be activated/coupled.

By “photosensitising agent” is meant any compound that falls within the definition of Formula I in the present application.

The term “aprotic solvent” means a solvent that has no OH groups and therefore cannot donate a hydrogen bond.

The term “handle” or “handle for conjugation” means a functional group that is suitable for covalently attaching the photosensitising agent to the carrier molecule. This may, for example, include a carboxylic acid group that may be converted to the corresponding activated succinimidyl ester, ready for conjugation to proteins or other carriers. Persons of skill in the art will appreciate that other activated functional groups may be suitable for conjugating the photosensitising agent to the carrier and therefore are included in the definition of “handle”.

Any documents referred to herein are hereby incorporated by reference. The listing or discussion of a prior-published document in this specification should not necessarily be taken as an acknowledgement that the document is part of the state of the art or is common general knowledge.

The invention will now be described in more detail by reference to the following, non-limiting, Figures and Examples.

### FIGURES

FIG. 1: Fluorescence of conjugated PPa-PEG and free, non-covalently bound PPa-PEG on a Nitrocellulose blot. The blot indicated that about 30% of the PPa PEG was non-covalently associated with the protein.

FIG. 2: Spectroscopic analysis of C6-PPaPEG conjugate and controls.

FIG. 3: Spectroscopic analysis of various photosensitisers in 2% DMSO/PBS.

FIG. 4: in vitro killing of SKOV3 cells by C6scFv-PPaPEG. The C6PPaPEG killed the C6 receptor expressing cells and spared the receptor negative cells.

FIG. 5: Biodistribution of PPa in SKOV3 tumour mice over a 24 hour period.

FIG. 6: Biodistribution of PPa-PEG in SKOV3 tumour mice over a 24 hour period.

FIG. 7: Biodistribution of Cationic PPa in SKOV3 tumour mice over a 24 hour period.

FIG. 8: Blood clearances of PPa and soluble PPa derivative photosensitisers.

FIG. 9: Blood clearances of C6-conjugated PPa and PPa-PEG and of free photosensitisers.

FIG. 10: Tumour uptake of C6-conjugated PPa and PPa-PEG and of free photosensitisers.

FIG. 11: Tumour:Blood ratio of C6-conjugated PPa and PPa-PEG and of free photosensitisers.

FIG. 12: PPa-PEG therapy in SKOV3 tumour mice.

FIG. 13: C6-PPa-PEG therapy in SKOV3 tumour mice.

FIG. 14: Comparison with Omniscan

FIG. 15: Cell kill profile of PPa (Pyropheophorbide-a). PPa was exposed to cells over a range of concentrations and exposed to light as described in the methods. Cell killing was measured using a MTT-based cell proliferation assay compared to untreated and fully lysed control cells. The results are plotted as percentage cell survival.

FIG. 16: Cell kill profile of 11. 11 was exposed to cells over a range of concentrations and exposed to light as described in the methods. Cell killing was measured using a MTT-based cell proliferation assay compared to untreated and fully lysed control cells. The results are plotted as percentage cell survival.

FIG. 17: Cell kill profile of 31C. 31C was exposed to cells over a range of concentrations and exposed to light as described in the methods. Cell killing was measured using a MTT-based cell proliferation assay compared to untreated and fully lysed control cells. The results are plotted as percentage cell survival.

FIG. 18: Cell kill profile of C6.5 scFv-compound II on SKOV3 tumour cells. A conjugate of C6.5 scFv-11 was exposed to cells over a range of concentrations (shown as net photosensitiser) and exposed to light as described in the methods. Cell killing was measured using a MTT-based cell proliferation assay compared to untreated and fully lysed control cells. The results are plotted as percentage cell survival.

FIG. 19: Confocal fluorescence microscopy of PPa-derived photosensitisers on SKOV3 cells

Left panel are white light transmission images, middle panel are photosensitiser fluorescence images, with the right panel showing cells magnified. Panels A-C(PPa), D-F (compound II) and G-1 (compound 31C). PPa and 11 show diffuse vesicular-intracellular staining whereas 31C shows punctuate staining.

FIG. 20: Confocal fluorescence microscopy of PPa-derived photosensitisers on SKOV3 cells, co-stained with bodipy ceramide

Left panel are white light transmission images, second panel are photosensitiser fluorescence images (red fluorescence), third panels show the bodipy ceramide co-stain (green fluorescence) with the right panel showing the overlaid images. Panels A-D (PPa), E-H (compound II) and I-L (compound 31C). D and H show significant yellow fluorescence indicating co-localisation

FIG. 21: Confocal fluorescence microscopy of PPa-derived photosensitisers on SKOV3 cells, co-stained with mitotracker

Left panel are white light transmission images, second panel are photosensitiser fluorescence images (red fluorescence), third panels show the mitotracker co-stain (green fluorescence) with the two right panels showing the overlaid images. Panels A-E (PPa), F-J (compound II) and K-O (compound 31C). D-E and I-J show significant yellow fluorescence indicating co-localisation

FIG. 22: Confocal fluorescence microscopy of PPa-derived photosensitisers on SKOV3 cells, co-stained with lysotracker

Left panel are white light transmission images, second panel are photosensitiser fluorescence images (red fluorescence), third panels show the lysotracker co-stain (green fluorescence) with the right panel showing the overlaid images. Panels A-D (PPa), E-H (compound II) and I-L (compound 31C). H shows a little yellow fluorescence indicating minor co-localisation whereas L shows significant yellow fluorescence indicating co-localisation

FIG. 23: Confocal fluorescence microscopy of PPa-derived photosensitisers on SKOV3 cells, co-stained with ER-tracker

Left panel are white light transmission images, second panel are photosensitiser fluorescence images (red fluorescence), third panels show the ER-tracker co-stain (green fluorescence) with the right panel showing the overlaid images. Panels A-D (compound II) and E-H (compound 31C). D shows significant yellow fluorescence indicating co-localisation.

FIG. 24: Skin photosensitivity of Foscan compared to compound II. Photosensitisers were administered at t=0 hrs and a laser shone on the skin after 1 hour.

The skin reaction was followed over time and the results were plotted according to the scale shown. Foscan showed grade-2 skin photosensitivity whereas 11 and its antibody conjugate

FIG. 25: UV/Visible spectrum of immunoconjugates

FIG. 26: A gel of C6-Mn(56) immunoconjugate shows the presence of the conjugate before dialysis

### EXAMPLE 1

### Development of New Derivatives of PPa

### Overview

The following example describes the development of a series of new derivatives of PPa, a hydrophobic photosensitiser, for conjugation to proteins. The approach involves the synthesis of a number of key intermediates which allow the preparation of porphyrins, chlorins and bacteriochlorins bearing a single amine or thiol reactive group and water solubilising groups which both act to suppress co-facial interaction (a likely mechanism for aggregation and precipitation in aqueous buffer) and reduce non-covalent binding to proteins.

In one example, a benzy ether unit with short tri(ethylene glycol) monomethyl ether (TEG) chains was attached to the propionic acid side chain of PPa making it very soluble in PBS and as this side chain also projects above the plane of the macrocycle self aggregation was also minimised. A hexynoic acid bio-conjugatable tether was attached to the 5-meso position of PPa through a metal catalysed cross-coupling reaction (Sonogashira Coupling) and activated by synthesising the succinimidyl ester derivative (active ester). Bioconjugation of this activated derivative was carried out in PBS/acetonitrile/DMSO to C6 (anti Her-2) scFv at up to 5 mg/ml of protein resulting in a highly active photoimmunoconjugate (PIC) containing 8-10 covalently attached photosensitisers. The resulting PIC demonstrates excellent in vitro cell kills, differentiating between targeted and non-targeted cells and minimal dark toxicity. The new PIC also displays improved pharmacokinetics resulting in rapid tumour uptake and higher tumour:blood ratios compared to the C6-PPa PIC which could be therapeutically very attractive with very little danger of skin photosensitivity. Overall, this translated into very effective killing of tumour cells in vivo with complete tumour regression being observed after 2 dose/2 light treatments in tumour bearing mice.

### Results and Discussion

The inventors have now shown that the design of a photosensitiser for covalent linking onto various antibody formats, but in particular single chain Fv's, to produce photoimmunoconjugates (PIC's) of high purity and potency for targeted photodynamic therapy (PDT), relies on a host of factors, including:

1. significant solubility in aqueous saline solutions, thereby avoiding intermolecular aggregation (and excited state quenching);

2. minimal non-specific (non-covalent) binding to proteins; and

3. incorporation of a single reactive group for conjugation, thereby avoiding cross-linking and the formation of product mixtures.

Water solubilising functional groups are, in general, divided into those that are charged (anionic or cationic) and those that are neutral, like oligo(ethylene glycol) (OEG) chains. The attachment of OEG chains on to porphyrins and other dyes have been shown to impart significant water solubility whilst their neutral character makes them easy to handle synthetically.

The initial water solubilising unit chosen in the present study was a benzy ether unit with short tri(ethylene glycol) monomethyl ether (TEG) chains. This functional group was synthesised according to literature procedures (as shown in scheme 1), and attached through an esterification of the propionic acid side chain of a modified pyropheophorbide-a derivative (7), scheme 2. Prior to attachment of the solubilising group, the vinyl side chain of commercially available methylpyropheophorbide a (MPPa, 5) was reduced (to prevent sidereactions) and subsequent hydrolysis of the propionic ester side chain in strong acid gave one of our key intermediates (7).

The presence of the propionic acid side chain allows the introduction of a large number of groups (both neutral and charged). The 5-meso position of derivative (8) was brominated in good yields using pyridinium perbromide. This enables introduction of a potential handle onto the macrocycle through metal-catalysed cross-coupling chemistry. Although it has previously been shown that one can carry out such chemistry on the 5-bromo derivative, we have established that the judicious choice of alkyne allows rapid and efficient coupling to this sterically crowded 5-bromo position, allowing the introduction of a large number of groups.

Commercially available 5-hexyn-oic acid was attached to the 5-meso position of derivative (9) through a copper-free Sonogashira coupling in high yields within 12 hours. Thin-layer chromatography indicated the presence of the desired alkyne (10) within a couple of hours. The last step involved converting the free carboxylic acid group of the attached side-chain into the corresponding activated succinimidyl ester with N-hydroxy succinimide and Dicyclohexyl carbodiimide (DCC). The resultant compounds were then ready for conjugation to proteins.

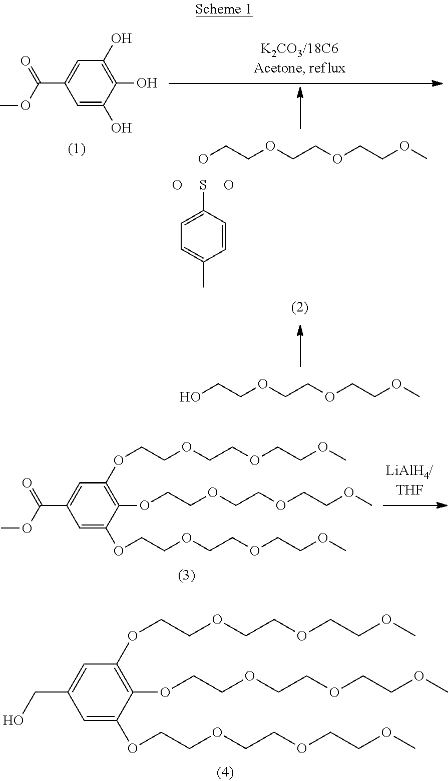

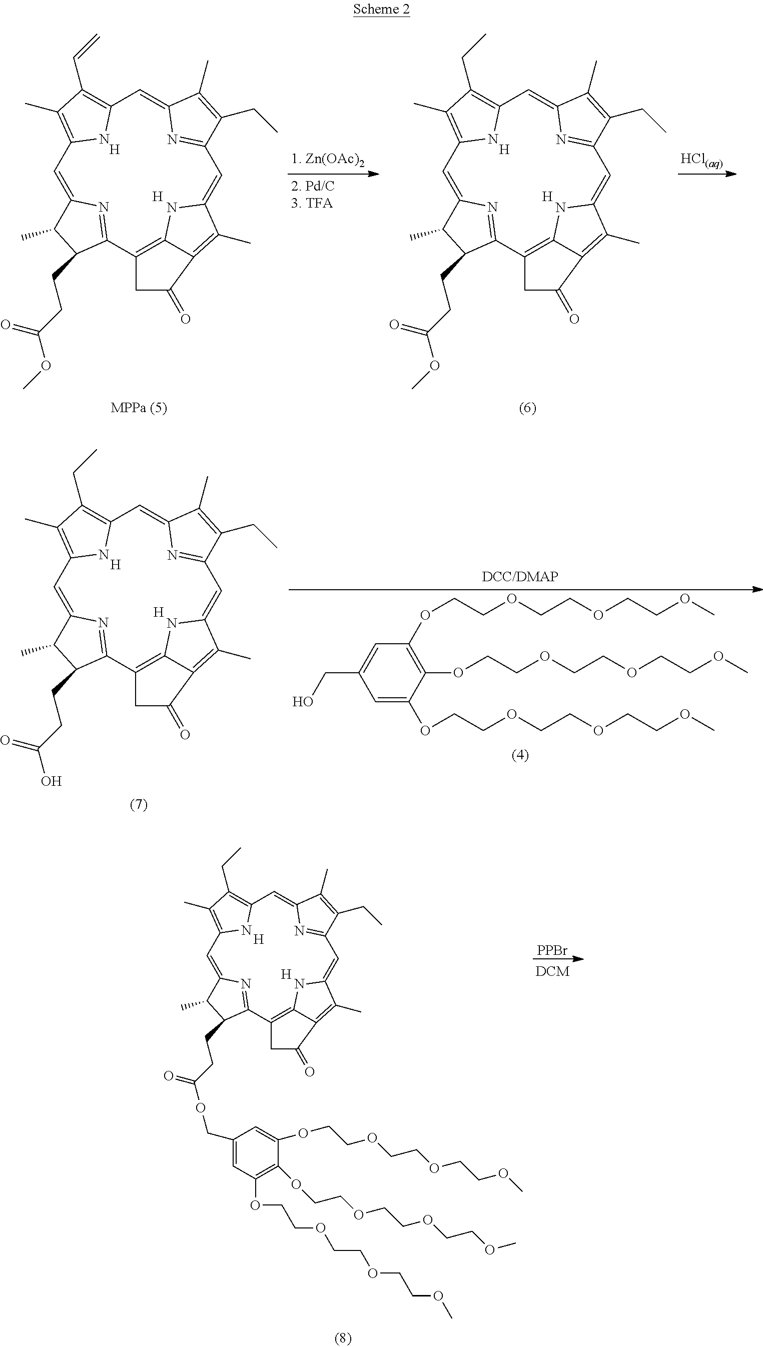

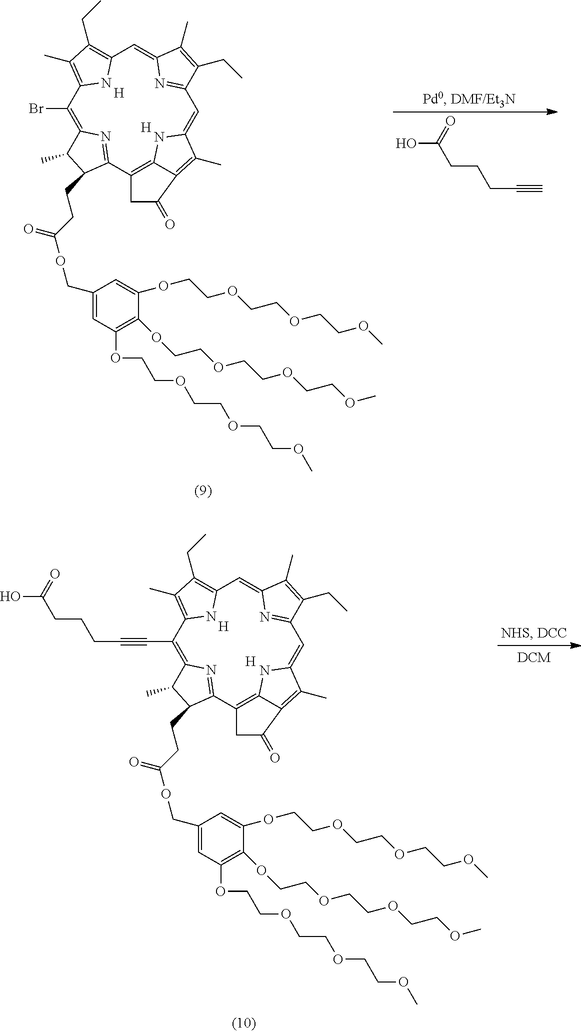

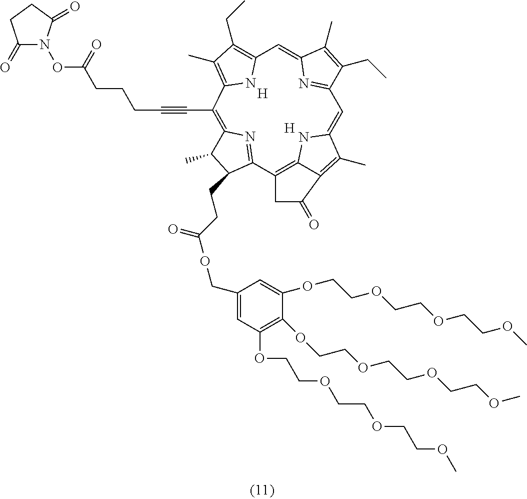

An alternative strategy was also employed for the production of the derivatives produced in scheme 2. By altering the sequence of bromination/hydrolysis outlined in scheme 2, the tri-PEGylated meso-5-bromo derivative (14) was obtained both in better yields and with less by-products (scheme 3).

The novel di-functional meso 5-bromo PPa derivative (13) is a key intermediate allowing the introduction of both solubilising groups and/or a conjugatable handle (structure A).

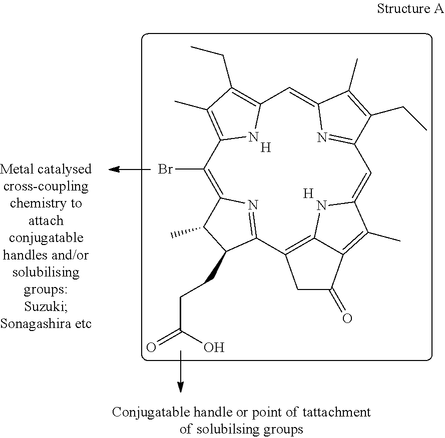

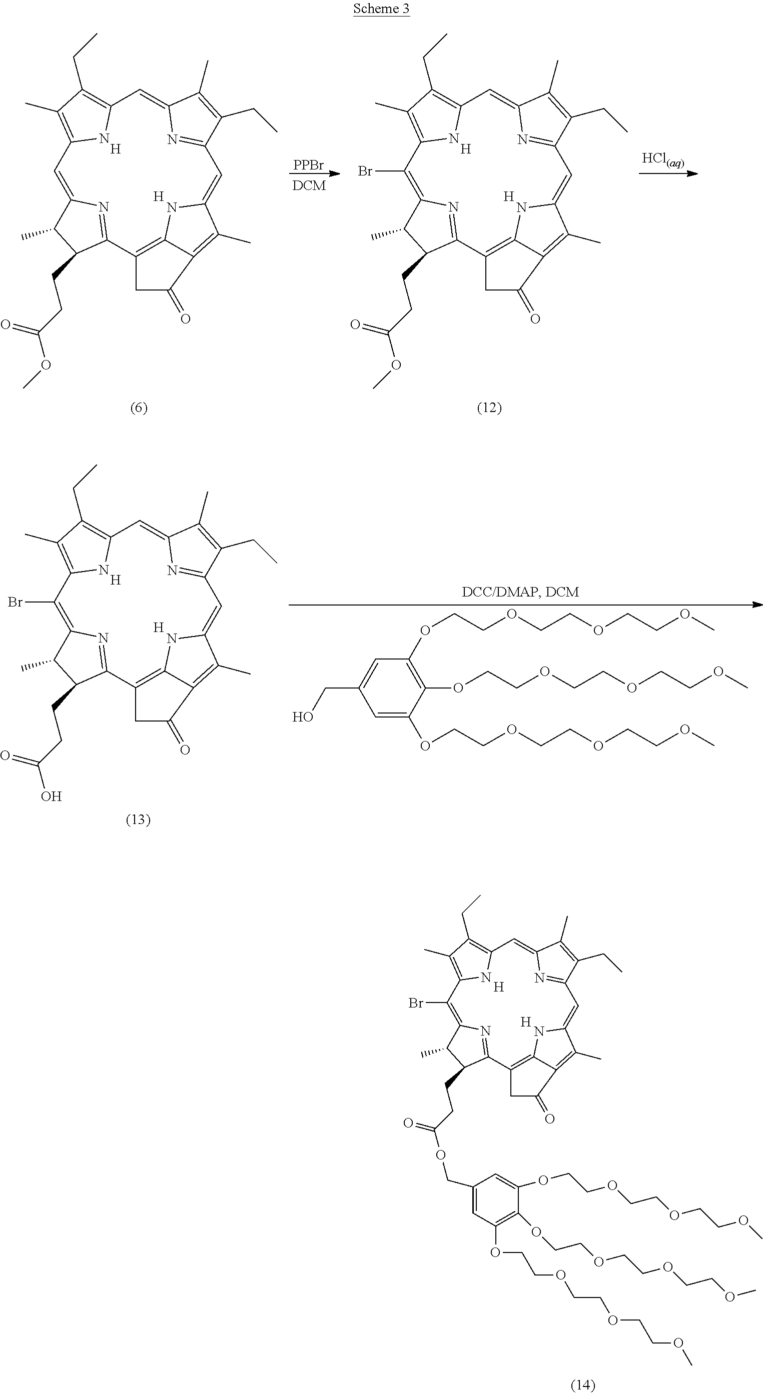

A further PPa derivative was synthesised by coupling the Hexyn-oic acid to the meso 5-brominated methyl ester derivative (12). The resulting acid (15) and the corresponding activated ester derivative (16) allowed us to carry out model studies to investigate the effect and efficiency of the coupling through the meso 5-position as compared to the propionic acid side chain and to quantify the solubilising effect of the tri-PEG side chain, scheme 4.

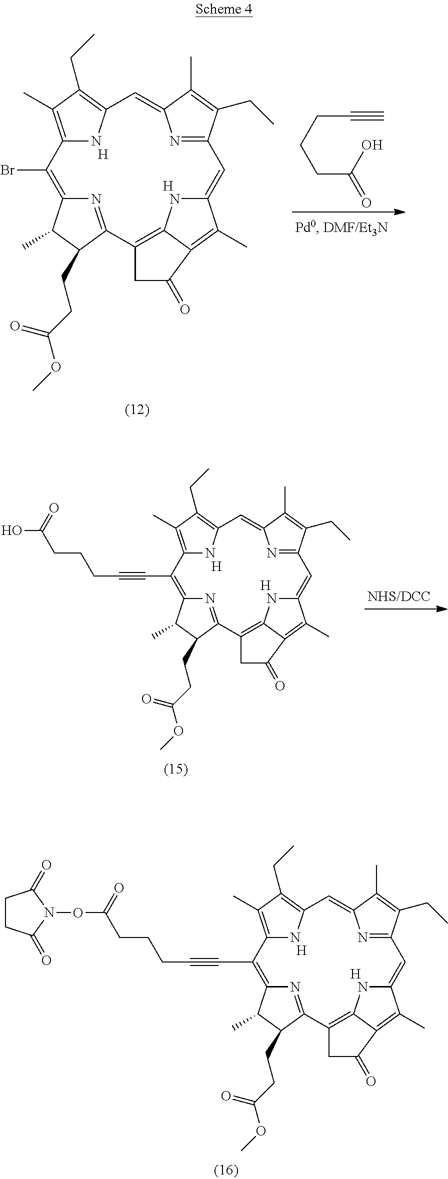

The availability of compound (13) enabled the synthesis of various cationic derivatives of PPa by reacting with a wide variety of substituted primary and secondary amines.

We found that the most efficient and high yielding procedure was to form the succinimidyl derivative (17) in situ by reacting compound (13) with N-hydroxysuccinimide using DCC in anhydrous DCM/THF (9:1). The presence of small amounts THF was critical in keeping everything in solution. The reaction was monitored by thin-layer chromatography and once complete conversion to the succinimidyl ester was observed, the desired amine was added in excess.

In one example the amine used was bis-[3-(dimethylamino)-propyl]amine, scheme 5. The desired amide (18) was obtained as a purple solid after chromatography on aluminium oxide (neutral, Brockmann grade 3). Quaternisation of the secondary amines to give the water soluble derivative (19) was achieved using excess methyl iodide in dry chloroform and isolated as a solid after trituration with dry ether. The hexyn-oic acid handle was attached through the metal catalysed coupling as described before to give compound (20).

In an alternative approach, the hexyn-oic acid was coupled at the meso 5-bromo position in (18) to give compound (21) which was then reacted with methyl iodide to give the dicationic derivative (22), scheme 6.

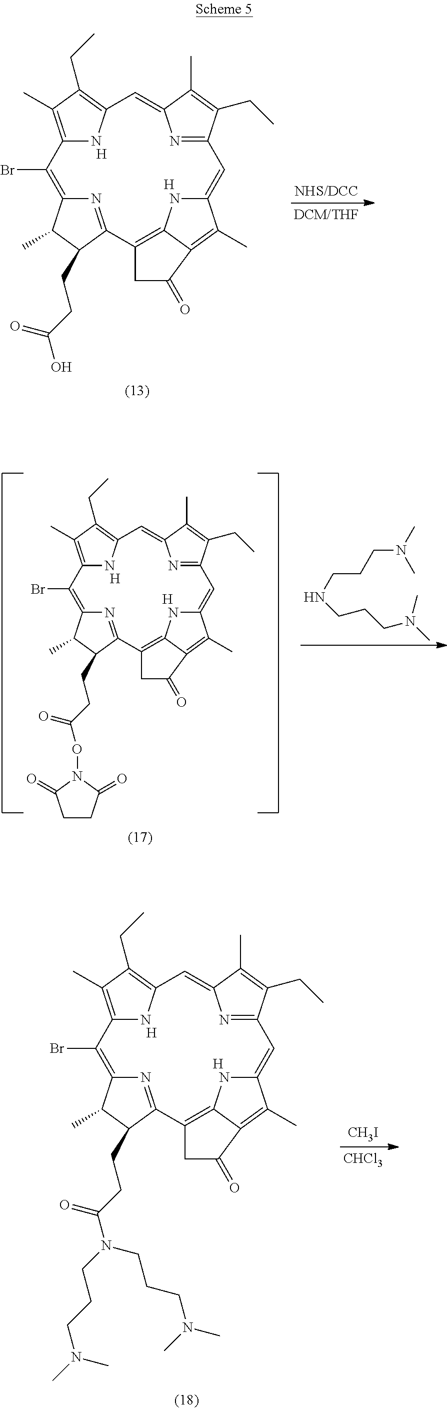

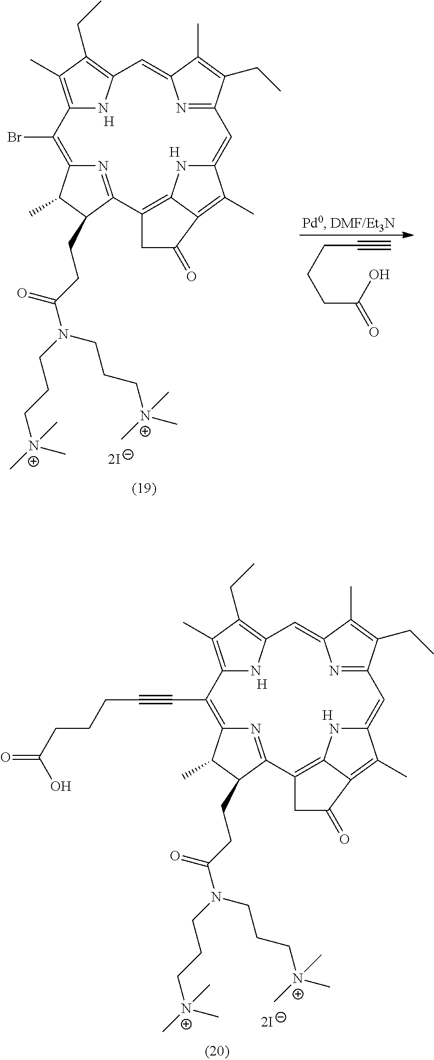

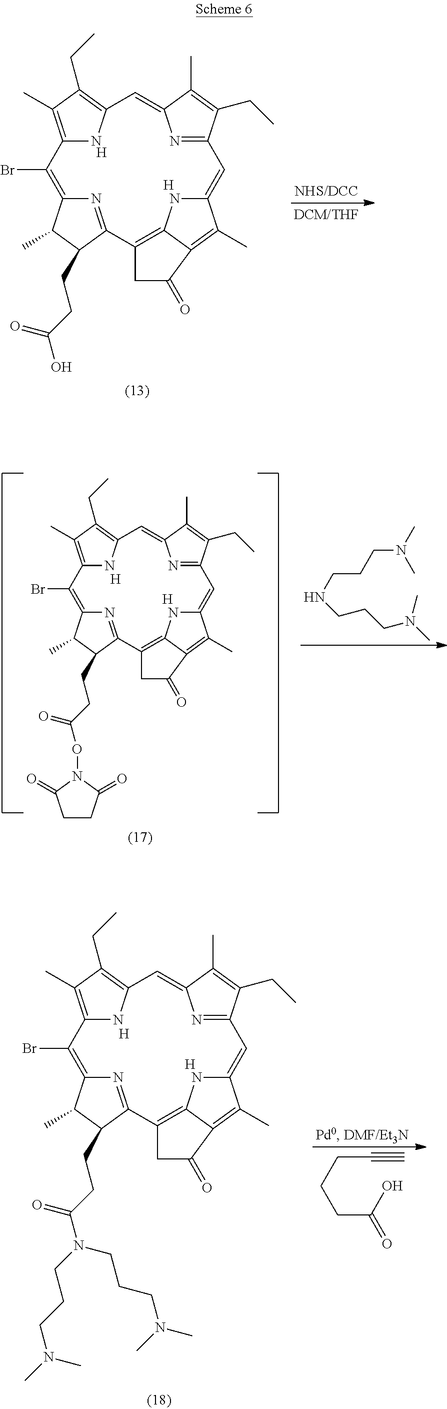

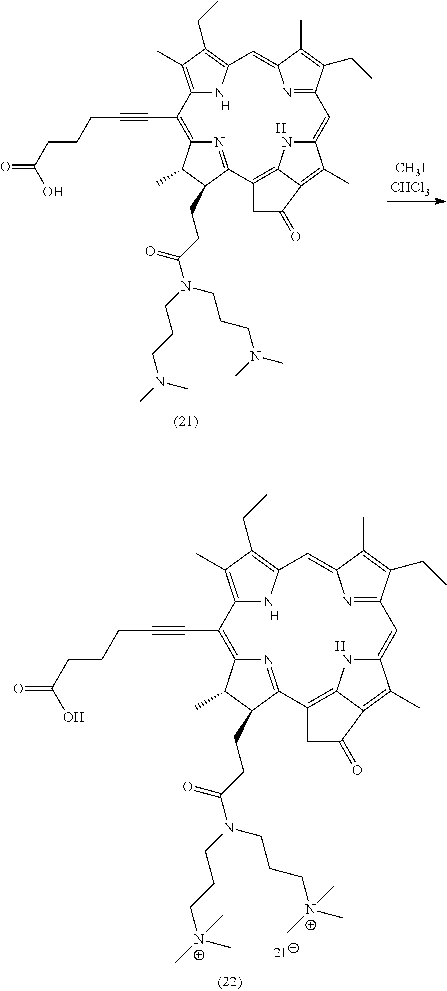

Chemistry was also developed in which we decided to retain the propionic acid side chain as the conjugatable handle and then solubilising groups were attached through derivatisation at the meso 5-bromo position of compound (12).

We begun by attaching a 4-pyridyl group through metal catalysed cross-coupling using 4-pyridyl boronic (Suzuki coupling) as reported in the literature, scheme 7. The pyridyl nitrogens can be readily quaternised, thus introducing a positive charge.

We initially looked at a simple quaternisation using methyl iodide in DMF at room temperature and we were also able to quaternise with short PEG chains in DMF at 80° C., schemes 7, 8 and 9.

The resultant mono-cationic derivatives are all soluble in water with compound (31) displaying the best solubility.

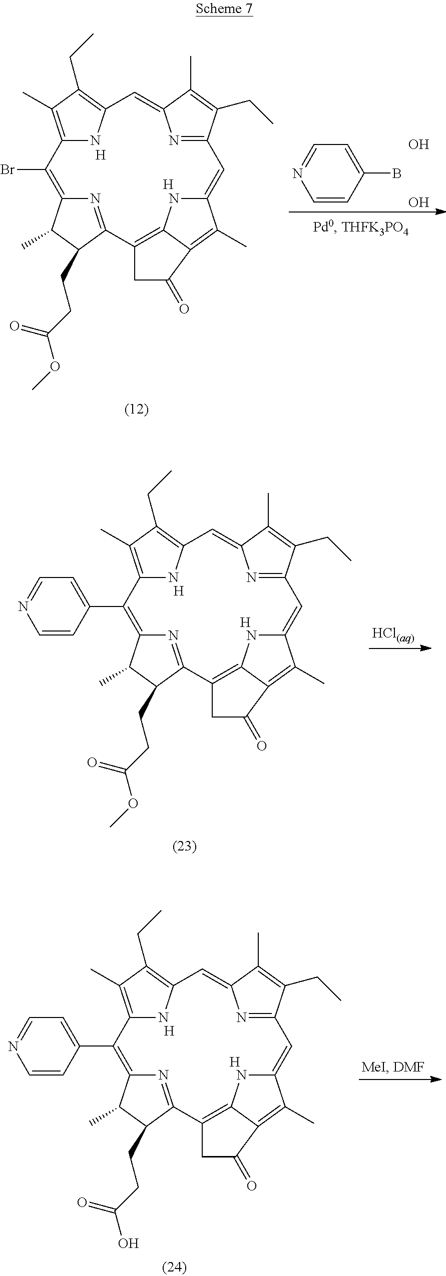

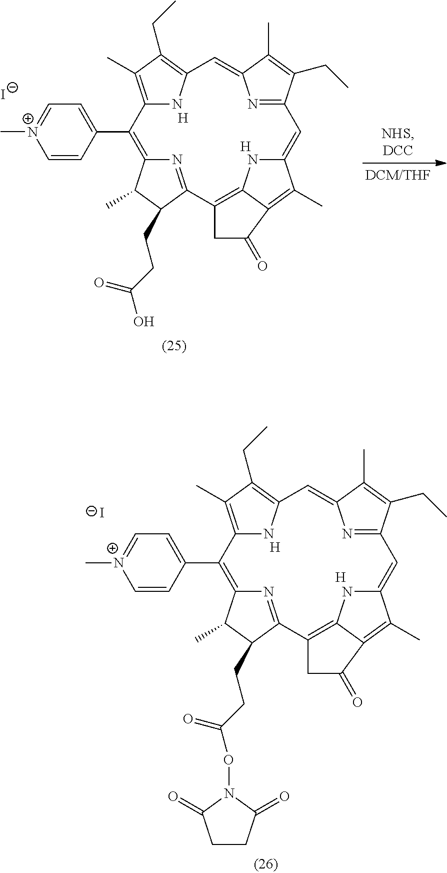

The alternative strategy in scheme 8 started well, but led to too many by-products during the hydrolysis step of the propyl ester side chain.

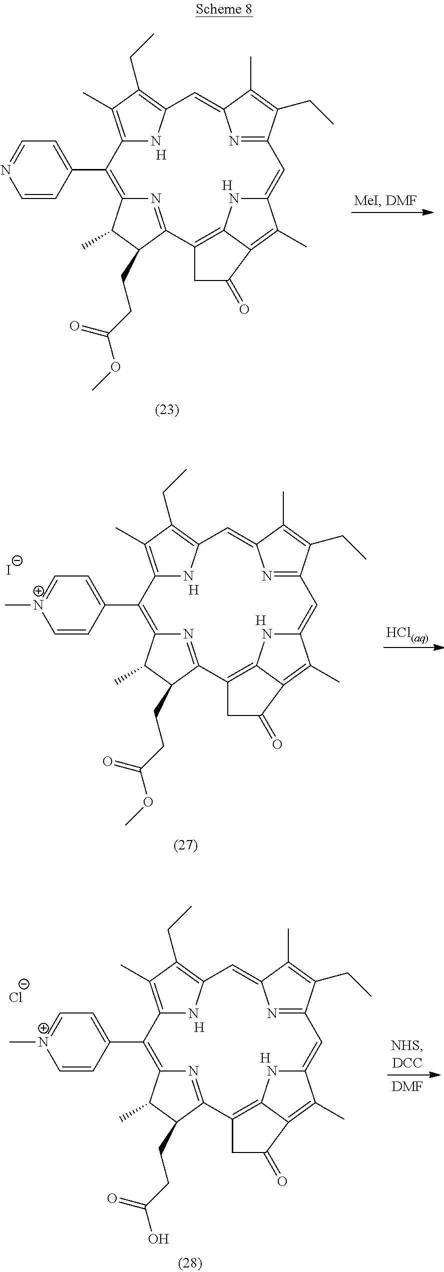

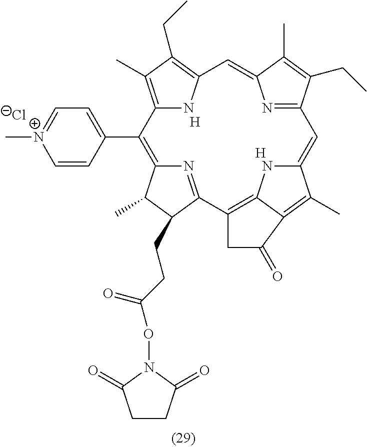

The quaternisation with the short PEG chain in scheme 9, lead to a small amount of the alkylated propionic acid side chain.

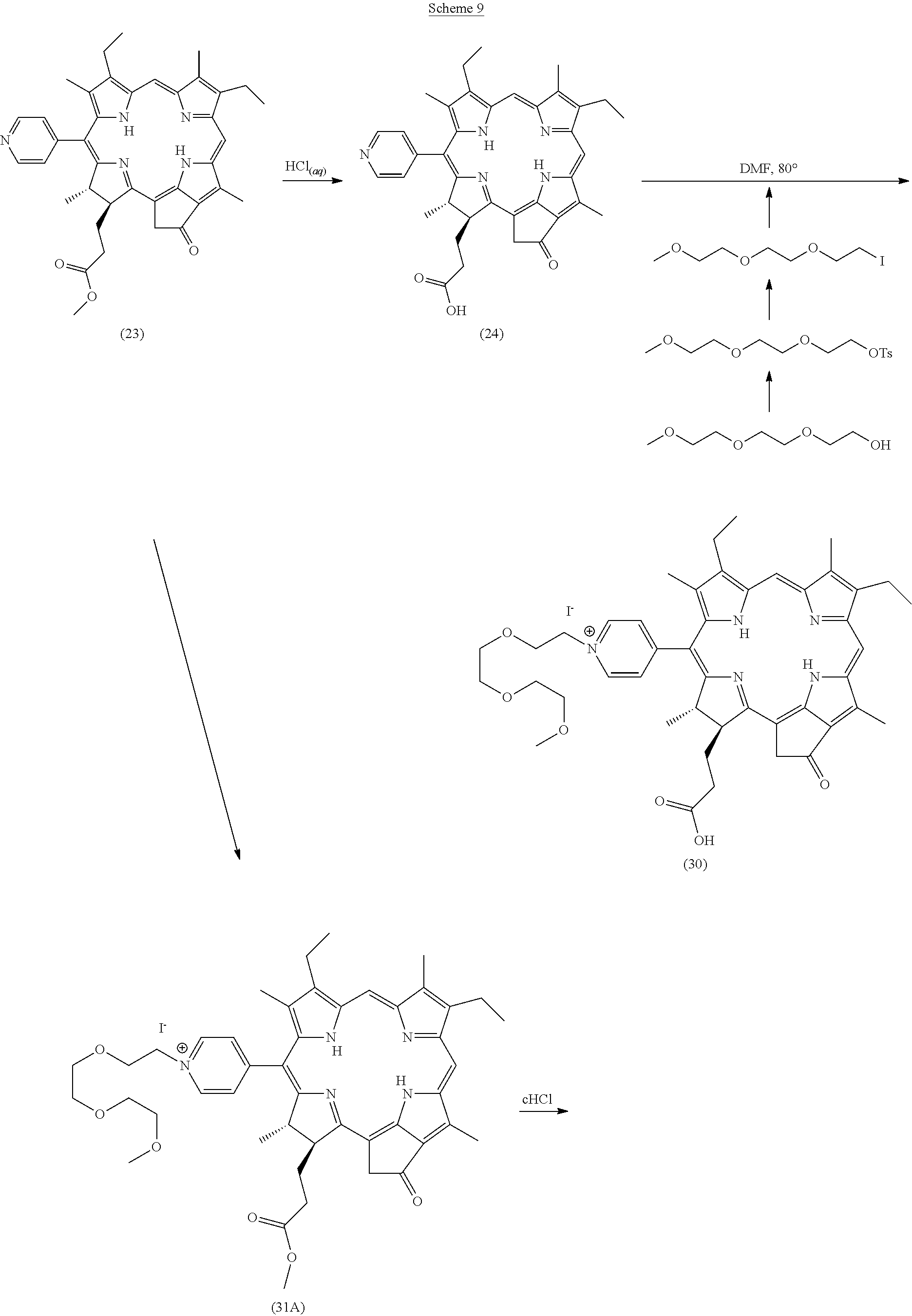

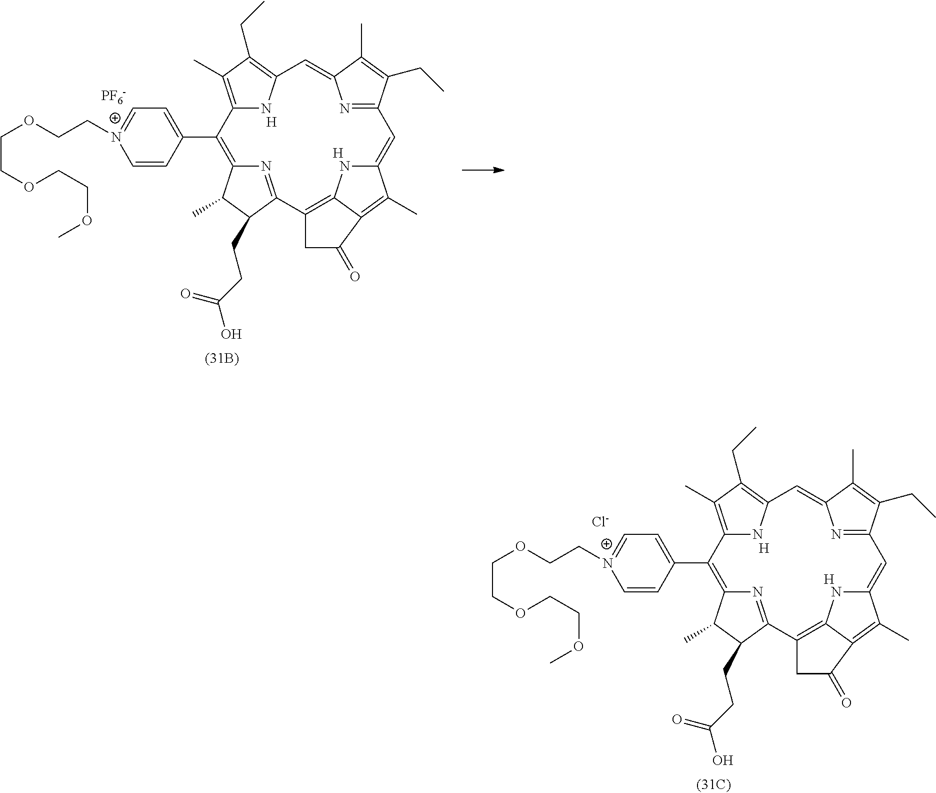

### Synthetic Routes and Methods

The manipulation of air and/or water sensitive compounds was carried out using standard Schlenk techniques. DCM and triethylamine were dried by distilling from CaH₂ and dry THF was obtained by distillation from sodium/benzophenone. All other reagents were used as supplied by commercial agents unless stated otherwise.

Analytical thin layer chromatography (TLC) was carried out on Merck glass backed silica gel 60 GF₂₅₄ plates or aluminium backed aluminium oxide (neutral) and visualisation when required was achieved using UV light or in some cases a chemical staining agent was used. Column chromatography was carried out on silica gel 60 or aluminium oxide(neutral or basic) deactivated with 5% water (referred to as Brockmann grade III) using a positive pressure of air. Where mixtures of solvents were used ratios reported are by volume.

NMR spectra were recorded at ambient probe temperature using a Bruker DPX400 (400 MHz). Chemical shifts are quoted as parts per million (ppm) with CDCl₃ as internal standard (for ¹H NMR, 7.26 ppm) and coupling constants (J) are quoted in Hertz (Hz). UV/Vis spectra were recorded on a Hewlett Packard 8450 diode array spectrometer. Mass spectra were carried out using a number of techniques and only molecular ions and major peaks are reported.

LCMS were run on a reverse phase C18 column, 2.1 mm diameter, 30 mm length, 3 micron particle size with a linear solvent gradient, going from 95% Water (0.1% formic acid): 5% MeCN to 5% Water: 95% MeCN over 10-15 mins.

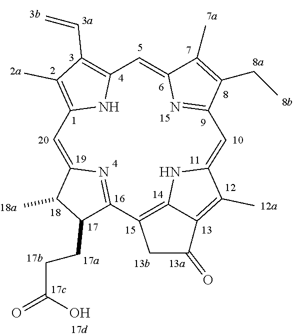 [SMILES: C=Cc1c(C)c2cc3nc(c4c5[nH]c(cc6nc(cc1[nH]2)C(C)=C6CC)c(C)c5C(=O)C4)[C@@H](CCC(=O)O)[C@@H]3C]

### Triethylene glycol monomethyl ether tosylate (2)

A solution of triethylene glycol monomethylether (25 g, 152 mmol) in THF (50 ml) was added dropwise to a stirred solution of sodium hydroxide (8.07 g, 201 mmol) in water (50 ml) under nitrogen while the temperature was maintained below 5° C. (ice-water-salt). Once addition was complete and at the same temperature, a solution of p-toluenesulfonyl chloride (24.78 g, 130 mmol) was added drop wise over 1 h. The reaction was quenched by pouring into water, DCM (200 ml) was added and the organic layer separated. The aqueous layer was back extracted with DCM (3×200 ml). The combined organic layers were washed with water (2×) and brine (2×), dried over MgSO₄, filtered and evaporated to a give (2) as a colourless oil (34 g, 82%). ¹NMR (400 MHz, CDCl₃) δ: 7.81 (d, J=8.2 Hz, 2H, Ar—H), 7.35 (d, J=7.9 Hz, 2H, Ar—H), 4.16 (t, J=4.7 Hz, 2H, CH₂), 3.57-3.82 (m, 10H, CH₂), 3.46, (s, 3H, O—CH₃), 2.44 (s, 3H, Ar—CH₃).

### 3,4,5-(triethylene glycol monomethyl ether) benzoate (3)

To a solution of (2) (24.51 g, 76.9 mmol) in acetone (220 ml), methyl-3,4,5-trihydroxybenzoate (4.5 g, 24.4 mmol), anhydrous potassium carbonate (16.85 g, 122 mmol) and 18-crown-6 (1.3 g, (4.88 mmol) were added. The resulting slurry was stirred and refluxed under argon for 2 days. The resulting light-brown reaction mixture was filtered to remove the insoluble inorganics and concentrated under vacuum to give a brown residue. This was re-dissolved in chloroform (500 ml) and washed with satd. sodium carbonate solution (5×500 ml), satd. sodium bicarbonate solution (3×250 ml) and finally with brine (250 ml). The organic phase was separated, dried over Na₂SO₄ and evaporated to give the crude material as a light-brown oil. This was purified by column chromatography on silica eluting with 5% MeOH in chloroform (R_{f} 0.4) to give (3) as a slight yellow oil (10.6 g, 70%). ¹NMR (400 MHz, CDCl₃) δ: 7.28 (s, 2H, Ar—H), 4.23-4.17 (m, 6H, CH₂), 3.89 (s, 3H, ArCO₂CH₃), 3.87, (t, J=4.9 Hz, 4H, O—CH₃), 3.80 (t, J=4.9

Hz, 2H, O—CH₃) 3.79-3.71 (m, 6H, CH₂), 3.69-3.63 (m, 12H, CH₂), 3.56-3.53 (m, 6H, CH₂), 3.39 (s, 9H, O—CH₃).

### 3,4,5-(triethylene glycol monomethylether) benzyalcohol (4)

To an ice-cooled stirred suspension of LiAlH₄ (0.86 g, 22.9 mmol) in anhydrous THF (35 ml), the ester (3) 8.95 g, 14.4 mmol) dissolved in anhydrous THF (85 ml) was added drop-wise over 1 h under argon. The reaction was allowed to warm to room temperature following the addition and stirred for after 6 h, a TLC (silica gel 10% MeOH/CHCl₃, R_{f} 0.56) at this point still showed the presence of starting material and a further portion of LiAlH₄ (0.86 g, 22.9 mmol) was added by cooling the reaction mixture down to below 5° C. Again after the addition the mixture was allowed to warm to room temperature and stirred for a further 6 h at which point all of the ester had been consumed. The reaction mixture was diluted by the addition of THF (300 ml) and small portions of a concentrated solution of Na₂SO₄.10H₂O in water and celite was added until the hydride was fully quenched. The reaction mixture was filtered and concentrated to give a slightly yellow clear oil 6.3 g, 93%, R_{f} 0.37). ¹NMR (400 MHz, CDCl₃) δ: 6.59 (s, 2H, Ar—H), 4.55 (s, 2H, Ar—CH₂), 4.23-4.17 (m, 6H, CH₂), 3.87 (t, J=4.9 Hz, 4H, O—CH₃), 3.80 (t, J=4.9 Hz, 2H, O—CH₃) 3.79-3.71 (m, 6H, CH₂), 3.69-3.63 (m, 12H, CH₂), 3.56-3.53 (m, 6H, CH₂), 3.4 (s, 9H, O—CH₃); MS (EI) 594 (M⁺)

### Methyl Mesopyropheophorbide a (6)

A solution of Zn(OAc)₂.2H₂O (1.27 g, 5.78 mmol)) in methanol (52 ml) was added to a solution of methylpyropheophorbide a 5 (1.2 g) in dichloromethane (90 ml). The mixture was stirred at room temperature under argon for 2 h and followed by UV/Vis spectroscopy. The reaction mixture was then washed with water (4×100 mL), back extracted with DCM and the combined organic layers was collected and dried over Na₂SO₄. The solvent was removed and the solid further dried on a high vacuum pump. The residue was dissolved in dry THF (110 ml), triethylamine (312 ml) followed by Pd/C (10%, 120 mg). The resultant mixture was hydrogenated (with a hydrogen balloon) at room temperature for 24 h and then filtered through a pad of Celite. The solvent was removed and the residue was treated with TFA (33 ml) for 2 h at room temperature under argon. The reaction mixture was quenched by carefully pouring it on to ice-water and extracted with DCM until the water layer was clear. The organic layers were combined and washed with water (2×200 ml) and 5% NaHCO₃ (1×200 ml) and dried over Na₂SO₄. The solvent was removed after filtration, and the residue was purified by column chromatography over neutral alumina (Brockmann grade III) eluting with 10% ethyl acetate/DCM to give the desired compound as a dark purple solid (1.17 g, 97%). R_{f} 0.66. ¹H NMR (CDCl₃, 400 MHz) 9.49 (s, α-meso), 9.23 (s, β-meso), 8.48 (s, δ-meso), 5.22 (q, 2H, CH₂CO), 4.49 (m, 1H, 8-H), 4.30 (m, 1H, 12-H), 3.85 (q, 2H), 3.71 (q, 2H, H-8), 3.69 (s, 3H, CO₂CH₃), 3.62 (s, 3H, 6-CH₃), 3.43 (s, 3H, 2-CH₃), 3.27 (s, 3H, 7-CH₃), 2.72 (m, 1H, 17a-CH₂), 2.6 (m, 1H, 17a-CH₂), 2.32 (m, 1H, 17b-CH₂), 1.82 (d, 3H, Me-13), 1.74 (t, 3H, Me-17-H), 1.71 (t, 3H, Me-4), 0.63 (br s, 1H, NH), −1.57 (br s, 1H, NH). MS (ESI) 551.3 (M+)

### Meso Pyropheophorbide a (7)

Concentrated hydrochloric acid (100 ml) was added in small portions to meso-methyl pyropheophorbide a (6) (0.20 g, 0.366 mmol) and stirred at room temperature, light protected and under argon for 2 h. The reaction mixture was poured into ice-water (600 ml) extracted with chloroform (3×100 ml). The combined organic layers were washed with 5% NaHCO₃ (1×100) and water (1×200) and dried (Na₂SO₄), filtered and the solvent removed to obtain (7) as a dark blue solid (0.17 g, 86%) R_{f} 0.24. ¹H NMR (CDCl₃, 400 MHz) 9.45 (s, α-meso), 9.19 (s, β-meso), 8.46 (s, δ-meso), 5.19 (q, 2H, CH₂CO), 4.48 (m, 1H, 8-H), 4.31 (m, 1H, 12-H), 3.84 (q, 2H, H-8), 3.70 (m, 2H), 3.65 (s, 3H, 6-CH₃), 3.30 (s, 3H, 2-CH₃), 3.26 (s, 3H, 7-CH₃), 2.72 (m, 1H, 17a-CH₂), 2.6 (m, 1H, 17a-CH₂), 2.32 (m, 1H, 17b-CH₂), 1.82 (d, 3H, Me-13), 1.74 (t, 3H, Me-17-H), 1.71 (t, 3H, Me-4), 0.63 (br s, 1H, NH), −1.57 (br s, 1H, NH). HSMS (ESI) m/z calcd. for C₃₃H₃₇N₄O₃ (M⁺) 537.3315: found: 537.2873

This was used without any further purification.

### Meso Pyropheophorbide a-3,4,5-(triethylene glycol monomethylether) benzyl ester (8)

DMAP (0.033 g, 0.273 mmol, catalytic) and DCC (0.078 g, 0.376 mmol) were added to a solution of meso pyropheophorbide a (7) (0.08 g, 0.149 mmol) in dry DCM (40 ml) followed by the benzyl alcohol 4 (0.13 g, 0.223 mmol) dissolved in dry DCM (10 ml) and the reaction mixture stirred room temperature, light protected, under nitrogen for 5 h. (R_{f} 0.58). The solvent was removed to obtain a dark solid. It was redissolved in DCM and washed with 0.5 M HCl (1×), water (2×) and dried over Na₂SO₄, filtered and concentrated to give crude (8). This was further purified by column chromatography on silica gel eluting with 5% MeOH/CHCl₃. A dark purple oil was obtained which was triturated with hexane to give (0.073 g, 44%). ¹H NMR (CDCl₃, 400 MHz) 9.48 (s, 1H, α-meso), 9.21 (s, 1H, β-meso), 8.46 (s, 1H, δ-meso), 6.51 (s, 2H, aromatic), 5.17 (q, 2H, CH₂CO), 4.94 (q, 2H) 4.47 (m, 1H, 8-H), 4.29 (m, 1H, 12-H), 4.08 (m, 6H), 3.85 (q, 2H,), 3.70 (m, 2H), 3.79-3.58 (m, 33H), 3.43-3.33 (m, 9H), 3.30 (s, 3H, 2-CH₃), 3.26 (s, 3H, 7-CH₃), 3.20 (m,) 0.64 (br s, 1H, NH), −1.6 (br s, 1H, NH). LC/MS water/methanol 10/90→90/10 over 10 mins. 1113.59 [M+] single peak.

### Meso 5-Bromopyropheophorbide a-3,4,5-(triethylene glycol monomethylether) benzyl ester (9)

To a solution of compound 8 (0.56 g, 0.5 mmol) in dry DCM (120 ml) pyridinium perbromide (0.21 g, 0.65 mmol) previously dried on a high vacuum pump and anhydrous pyridine (530 μl) were added and the reaction mixture stirred at room temperature under argon. The reaction was monitored by UV/Vis and was complete by 30 min. at which point the solvent was removed and crude material immediately purified by chromatography on silica gel eluting with 5% MeOH/CHCl₃ to give the desired compound as a purple viscous oil (0.41 g, 69%). ¹H NMR (CDCl₃, 400 MHz) 9.56 (s, 1H, α-meso), 9.45 (s, 1H, β-meso), 6.50 (s, 2H, aromatic), 5.21 (d, 2H, CH₂CO), 4.94 (q, 2H) 4.91 (m, 2H), 4.47 (m, 1H, 8-H), 4.29 (m, 1H, 12-H), 3.80-3.49 (m, 33H), 3.39 (s, 3H), 3.37 (s, 2H), 3.36 (s, 3H), 3.30 (s, 2H), 2.51 (m,), 2.23 (m), 0.64 (br s, 1H, NH), −1.6 (br s, 1H, NH). MS (ESI) 1193.6 (M+1); UV/Vis (DCM)

### Methyl meso 5-bromopyropheophorbide a (12)

Pyridinium perbromide (0.2 g, 0.618 mmol) previously dried under high vacuum and anhydrous pyridine (500 μl) were added to a solution of meso-methylpyropheophorbide a (6) (0.27 g, 0.492 mmol) in dry DCM (75 ml). The reaction mixture was stirred for 20 mins at room temperature, light protected and under argon. The reaction was monitored by UV/Vis and once complete, the solvent was removed to give the crude product as a brown solid. This was purified by column chromatography on neutral aluminium oxide (Brockmann III) using DCM as the eluent. R_{f} 0.82 (5% MeOH/CHCl₃). Final product was obtained as a purple solid (0.24 g, 77%) ¹H NMR (CDCl₃, 400 MHz) 9.56 (s, α-meso), 9.45 (s, β-meso), 5.0 (q, 2H, CH₂CO), 4.89 (m, 1H, 8-H), 4.25 (m, 1H, 12-H), 3.85 (q, 2H, H-16), 3.69 (m, 2H, CH₂CH₃-13-H), 3.68 (s, 3H, Me-6-H), 3.60 (s, 3H, OMe), 3.56 (s, 3H, Me-15-H), 3.3 (s, 3H, 2-H), 2.6 (m, 2H, 9-H), 2.2 (m, 1H, 10-Ha), 1.78 (m, 1H, 10-Hb), 1.70 (d, 3H, Me-13), 1.6 (t, 3H, Me-17-H), 1.27 (t, 3H, Me-4), 0.9 (br s, 1H, NH), −1.72 (br s, 1H, NH). MS ES: 629.2, 631.2 [M, M+2]

### Meso 5-Bromopyropheophorbide a (13)

Methyl meso-bromopyropheophorbide a (0.97 g, 1.54 mmol) was slowly dissolved in concentrated hydrochloric acid (200 ml) and stirred at room temperature, under argon for 5 h. The reaction was quenched by slowly pouring the reaction mixture onto stirred ice-water mixture and extracted exhaustively with chloroform until the aqueos layer was clear. The combined organic layers were washed with satd. NaHCO₃, water, dried over Na₂SO₄ and evaporated to give a purple solid (0.65 g, 70%) R_{f} 0.16 (5% MeOH/CHCl₃). ¹H NMR (CDCl₃, 400 MHz) 9.56 (s, α-meso), 9.45 (s, β-meso), 5.0 (q, 2H, CH₂CO), 4.89 (m, 1H, 8-H), 4.25 (m, 1H, 12-H), 3.85 (q, 2H, H-16), 3.69 (m, 2H, CH₂CH₃-13-H), 3.68 (s, 3H, Me-6-H), 3.60 (s, 3H, OMe), 3.56 (s, 3H, Me-15-H), 3.3 (s, 3H, 2-H), 2.6 (m, 2H, 9-H), 2.2 (m, 1H, 10-Ha), 1.78 (m, 1H, 10-Hb), 1.70 (d, 3H, Me-13), 1.6 (t, 3H, Me-17-H), 1.27 (t, 3H, Me-4), 0.9 (br s, 1H, NH), −1.72 (br s, 1H, NH). HSMS (ESI) m/z calcd. for C₃₃H₃₆N₄O₃Br (M+1) 615.1971: found: 615.1987

### Meso 5-Bromopyropheophorbide a-3,4,5-(triethylene glycol monomethylether) benzyl ester (14)

Meso-bromopyropheophorbide a (13) (0.08 g, 0.13 mmol) was dissolved in dry DCM/10% THF (10 ml) and placed under a blanket of argon. To this stirred solution a solution of the tripeg benzyl alcohol (4) (0.12 g, 0.195 mmol) dissolved in the minimum of dry DCM was added followed by DCC (0.17 g, 0.84 mmol), DMAP (0.062 g, 0.506 mmol) and DPTS (0.14 g, 0.48 mmol). The resulting mixture was shielded from light and stirred for 10 min. before the addition of N-ethyldiisopropylamine (79 μl, 0.46 mmol) and stirring was continued for a further 6 h when TLC (silica gel) confirmed the consumption of all the starting material. The reaction mixture was diluted with DCM (200 ml), washed with satd. solution of ammonium chloride (100 ml) and water (5×100 ml), the organic layer was separated and the aqueous layer back extracted with DCM. The combined DCM washings were dried over Na₂SO₄ and evaporated to give a dark purple-brown oil. This was purified by column chromatography (silica gel 5% MeOH/chloroform, R_{f} 0.46), the relevant fractions were combined and evaporated to give an oil. This was further purified by dissolving in the minimum of chloroform, layering the solution with hexane and leaving it overnight at 5° C. The resulting white precipitate was filtered off and the procedure repeated until no further precipitation was observed. The desired compound (14) was obtained as a dark purple viscous oil (0.11 g, 73%). ¹H NMR (CDCl₃, 400 MHz) 9.56 (s, 1H, α-meso), 9.45 (s, 1H, β-meso), 6.50 (s, 2H, aromatic), 5.21 (d, 2H, CH₂CO), 4.94 (q, 2H) 4.91 (m, 2H), 4.47 (m, 1H, 8-H), 4.29 (m, 1H, 12-H), 3.80-3.49 (m, 33H), 3.39 (s, 3H), 3.37 (s, 2H), 3.36 (s, 3H), 3.30 (s, 2H), 2.51 (m,), 2.23 (m), 0.64 (br s, 1H, NH), −1.6 (br s, 1H, NH). MS (ESI) 1193.6 (M+1);

### Meso 5-Ethynyihexanoic acid pyropheophorbide a-3,4,5-(triethylene glycol monomethylether) benzyl ester (10)

The 5-Bromo derivative (14) was dissolved (0.1 g, 0.0837 mmol) in a mixture of dry and deoxygenated DMF/EtN₃ (2 ml, 10:1). To this stirred solution under argon, tri-(o-tolyl)phosphine (0.03 g, 0.0973 mmol) followed by tris(dibenzylideneacetone) dipalladium (0) (0.012 g, 0.0127 mmol) was added followed by a large excess of the 5-hexynoic acid (170 μl, 1.677 mmol). The resulting dark purple solution was purged with argon and placed under an argon atmosphere and stirred at room temperature, shielded from light. The reaction was monitored by TLC (silica gel 10% MeOH/CHCl₃) and the product could be observed within 2-4 h as a dark blue spot on the plate which also had a red fluorescence under illumination with long wavelength light, R_{f} 0.5, the starting material R_{f} 0.69 does not fluoresce due to the presence of the bromine atom. The reaction was left to stir for 12 h, diluted with diethyl ether and washed with a mixture of water/citric acid solution, the organic layer was separated and the aqueous layer back extracted with ether. The combined ether extracts were then dried over Na₂SO₄ and evaporated to give a dark purple oil which was purified by chromatography (silica gel 5% MeOH/CHCl₃) to give the desired compound as a purple viscous oil (0.07 g, 70%). ¹H NMR (CDCl₃, 400 MHz) 9.40 (s, 1H, α-meso), 9.24 (s, 1H, β-meso), 6.48 (s, 2H, aromatic), 5.21 (d, 2H, CH₂CO), 4.94 (d, 2H) 4.75 (m, 2H), 4.07 (m, 1H, 8-H), 3.91-3.36 (m, 35H), 3.36 (s, 3H), 3.34 (s, 2H), 3.24 (s, 3H), 3.06 (m, 2H), 2.51 (m,), 2.23 (m), 0.64 (br s, 1H, NH), −1.05 (br s, 1H, NH). HRMS (ESI) m/z cald. for C₆₇H₉₁N₄O₁₇ (M+1) 1223.6379 Found 1223.6400; UV/Vis (DCM) UV/Vis (DCM) 417, 524, 558, 614, 673.

### Meso 5-Ethynyihexanoyl succinimido ester pyropheophorbide a-3,4,5-(triethylene glycol monomethylether) benzyl ester (11)

To a solution of the meso 5-ethynyl hexanoic acid PPa derivative (10) (0.03 g, 0.0245 mmol) in dry DCM (5 ml), N-hydroxy succinimide (3.7 mg, 0.0319 mmol) and DCC (7.6 mg, 0.0368 mmol) were added and the resulting mixture stirred for 12 h. Silica gel (5% MeOH/CHCl₃, R_{f} 0.38). The solvent was evaporated and the residue purified by column chromatography to give the desired compound as a dark purple viscous oil. This was dissolved in a minimum amount of dry DCM and sufficient hexane was added and the resulting solution left at 5° C. overnight, filtered to remove dicyclohexyl urea and evaporated and dried (0.023 g, 71%). ¹H NMR (CDCl₃, 400 MHz) 9.40 (s, 1H, α-meso), 9.24 (s, 1H, β-meso), 6.48 (s, 2H, aromatic), 5.21 (d, 2H, CH₂CO), 4.94 (d, 2H) 4.75 (m, 2H), 4.07 (m, 1H, 8-H), 3.91-3.36 (m, 35H), 3.36 (s, 3H), 3.34 (s, 2H), 3.24 (s, 3H), 3.25 (m, 4H, succinimidyl) 3.06 (m, 2H), 2.51 (m,), 2.23 (m), 0.64 (br s, 1H, NH), −1.05 (br s, 1H, NH). HRMS (ESI) m/z cald. for C₇₁H₉₃N₅O₁₉ (M+Na) 1342.6254 Found 1342.6378; LCMS confirms a single component with the correct mass; UV/Vis (DCM) UV/Vis (DCM) 417, 524, 558, 614, 673.

### Meso 5-Ethynylhexanoic acid methylpyropheophorbide a (15)

Methyl meso 5-bromopyropheophorbide a (12) was dissolved (0.03 g, 0.0477 mmol) in a mixture of dry and deoxygenated DMF/EtN₃ (2 ml, 10:1). To this stirred solution under argon, tri-(o-tolyl)phosphine (0.017 g, 0.0552 mmol) followed by tris(dibenzylideneacetone) dipalladium (0) (0.0066 g, 0.00722 mmol) was added followed by a large excess of the 5-hexynoic acid (97 μl, 0.953 mmol). The resulting dark purple solution was purged with argon and placed under an argon atmosphere and stirred at room temperature, shielded from light. The reaction was left to stir for 12 h, diluted with diethyl ether and washed with a mixture of water/citric acid solution, the organic layer was separated and the aqueous layer back extracted with ether. The combined ether extracts were then dried over Na₂SO₄ and evaporated to give a dark purple oil which was purified by chromatography (silica gel 2% MeOH/CHCl₃) to give the desired compound as a purple solid (0.023 g, 75%). ¹H NMR (CDCl₃, 400 MHz) 9.37 (s, 1H, α-meso), 9.24 (s, 1H, β-meso), 5.15 (d, 2H, CH₂CO), 4.71 (q, 2H), 4.12 (m, 1H, 8-H), 3.71 (m, 2H), 3.62 (s, 3H), 3.60 (s, 2H), 3.46 (s, 3H), 3.08 (m, 2H), 2.97 (m,), 2.75 (m), 2.23 (m), 0.64 (br s, 1H, NH), −1.05 (br s, 1H, NH). HRMS (ESI) m/z cald. for C₄₀H₄₅N₄O₅ (M+1) 661.3390 Found 669.3392;; UV/Vis (DCM) UV/Vis (DCM) 417, 524, 558, 614, 673.

### Meso 5-Ethynyihexanoyl succinimido ester acid methylpyropheophorbide a (16)

To a solution of the Meso 5-Ethynylhexanoic acid methylpyropheophorbide a (15) (0.02 g, 0.0303 mmol) in dry DCM (2 ml), N-hydroxy succinimide (4.2 mg, 0.0363 mmol) and DCC (7.5 mg, 0.0363 mmol) were added and the resulting mixture stirred for 12 h. The solvent was evaporated and the residue purified by column chromatography to give the desired compound as a dark purple solid (0.016 g, 70%). ¹H NMR (CDCl₃, 400 MHz) 9.37 (s, 1H, α-meso), 9.24 (s, 1H, β-meso), 5.15 (d, 2H, CH₂CO), 4.71 (q, 2H), 4.12 (m, 1H, 8-H), 3.71 (m, 2H), 3.62 (s, 3H), 3.60 (s, 2H), 3.46 (s, 3H), 3.08 (m, 2H), 3.01 (m, 4H, succinimidyl), 2.97 (m,), 2.75 (m), 2.23 (m), 0.64 (br s, 1H, NH), −1.05 (br s, 1H, NH). HRMS (ESI) m/z cald. for C₄₄H₄₈N₅O₇ (M+1) 758.3554 Found 758.3552; UV/Vis (DCM) 417, 524, 558, 614, 673.

### N-Bis-13-(dimethylamino)propylpmeso 5-Bromopyropheophorbide a amide (18)

Meso-bromopyropheophorbide a (13) (0.1 g, 0.163 mmol) was dissolved in dry DCM/20% THF (10 ml) and placed under a blanket of argon. To this stirred solution N-hydroxysuccinimide (0.0224 g, 0.0195 mmol) followed by DCC (0.0377 g, 0.0195 mmol) were added and the resulting mixture stirred at room temperature, shielded from light for 18 h, when TLC (5% MeOH/CHCl₃, R_{f} 0.48) showed the presence of the product and consumption of starting material. At this point excess bis-[3-(dimethylamino)propyl]amine (73 p1, 0.325 mmol) dissolved in a small volume of dry DCM was added and stirring continued for a further 12 h. TLC (silica gel (5% MeOH/CHCl₃) indicated that all of the active ester derivative (17) which had been generated in situ had been consumed and TLC (neutral aluminium oxide 10% MeOH/CHCl₃) showed the presence of a new major component. The reaction mixture was evaporated to give a purple viscous oil. This was purified by column chromatography on (neutral aluminium oxide Brockmann III) and the major fraction collected and evaporated to give the derivative (18) as a purple solid (0.1 g, 64%). ¹H NMR (CDCl₃, 400 MHz) 9.52 (s, 1H, α-meso), 9.42 (s, 1H, β-meso), 5.29 (d, 2H, CH₂CO), 4.93 (q, 2H), 4.34 (m, 1H, 8-H), 3.91 (m, 2H), 3.70 (m, 1H), 3.68 (s, 3H), 3.57 (s, 2H), 3.32 (s, 3H), 3.08 (m, 2H), 2.97 (m,), 2.75 (m), 2.23 (m), 0.64 (br s, 1H, NH), −1.73 (br s, 1H, NH). HRMS (ESI) m/z cald. for C₄₃H₅₈N₇O₂Br (M+1) 784.3914 Found 784.3900; UV/Vis (MeOH) 410, 514, 547, 610, 669.

### N-Bis-[3-(trimethylamino)propyl]-meso 5-Bromopyropheophorbide a amide diiodide (19)

The bis-amine (18) (0.02 g) was dissolved in dry chloroform and placed under a blanket of argon. To this solution excess methyl iodide (0.3 ml) was added and the reaction mixture stirred for 12 h. The solvent was evaporated and the oily residue triturated with dry diethyl ether (5×), each time the ether was carefully decanted off and a fresh batch added and finally the residue was dried under a high vacuum pump to give a purple sticky solid. HRMS (ESI) m/z cald. for C₄₅H₆₄N₇O₂Brl (M-I) 940.3350 Found 940.3367; UV/Vis (MeOH) 410, 514, 547, 610, 668. This compound is readily soluble in water and methanol.

### N-Bis-[3-(trimethylamino)propyl]-meso 5-Ethynyihexanoic acid pyropheophorbide a amide (21)

The 5-Bromo amide derivative (18) was dissolved (0.067 g, 0.00854 mmol) in a mixture of dry and deoxygenated DMF/EtN₃ (2 ml, 10:1). To this stirred solution under argon, tri-(o-tolyl)phosphine (0.03 g, 0.0973 mmol) followed by tris(dibenzylideneacetone) dipalladium (0) (0.012 g, 0.0127 mmol) was added followed by a large excess of the 5-hexynoic acid (170 μl, 1.677 mmol). The resulting dark purple solution was purged with argon and placed under an argon atmosphere and stirred at room temperature, shielded from light for 18 h, TLC (neutral aluminium oxide, (10% MeOH/CHCl₃) showed the consumption of all of the starting material. The reaction mixture was evaporated to dryness to give a dark purple viscous oil. This loaded onto a column of neutral aluminium oxide (Brockmann III) and eluted with 20% MeOH. Some minor porphyrin fractions eluted but the main band stayed on top of the column. This was scraped off and stirred in MeOH/CHCl₃ (1:1) and the dark purple solution was evaporated to give a purple viscous oil. HRMS (ESI) m/z cald. for C₄₉H₆₅N₇O₄B (M+1)) 816.5098 Found 816.5172; UV/Vis (THF) 416, 523, 558, 614, 673.

### 3-devinyl-20-pyridyl-methylpyropheophorbide-a (23)

3-devinyl-20-bromo-methylpyropheophorbide-a (0.2 g, 0.32 mmol) and 4-pyridyl boronic acid (0.39 g, 3.18 mmol) were degassed with dry nitrogen for 15 mins before adding dry THF (80 ml) and degassing for a further 30 mins with the nitrogen bubbling through. Pd(PPh₃)₄ (−80 mg) was added and continued degassing the mixture for 15 mins. K₃PO₄ (1.35 g, 6.35 mmol) was added and the reaction was heated under reflux for 15 hrs under nitrogen, light protected. Pd(PPh₃)₄ (−40 mg) was added and continued heating under reflux for 9 hours before diluting the mixture with chloroform and washing with water, sat NaHCO₃, brine and water and drying over Na₂SO₄. The crude product was purified on silica gel eluting with CHCl₃/THF (9/1) to obtain a dark oil which was recrystallised from CHCl₃/Hexane at 4° C. overnight to obtain long purple crystals (63% yield).

Rf (CHCl₃/THF): 0.3. ¹H NMR (CDCl₃, 400 MHz, 25° C.) 9.55 (1H, s, δ-meso H), 9.43 (1H, s, 10-meso H) 9.07 (1H, d, J=4.92 Hz, 20b), 8.9 (1H, d, J=4.92 Hz, 20b′), 8.15 (1H, d, J=4.72 Hz, 20a), 7.67 (1H, d, J=4.7 Hz, 20a′), 5.21 (2H, s, 13b-CH₂CO), 4.23 (1H, q, 18-H), 4.12 (1H, dd, 17 CH), 3.84 (2H, q, 3a-CH₂), 3.75 (2H, m, 8a CH₂CH₃), 3.7 (3H, s, 12a), 3.58 (3H, s, 17d) 3.31 (3H, s, 2a), 2.55 (2H, m, 17a), 2.37 (3H, s, 7a), 2.21 (2H, m, 17b), 1.75 (3H, t, 8b CH₃CH₂), 1.67 (3H, t, 3b CH₃CH₂), −1.45 (1H, s, NH), MS ES (m/z) (Calc. for C₃₉H₄₁N₅O₃ 627.32) Found 628.32 M⁺+H. LCMS (C₁₈) retention time 11.12 min (628.3306) UV/Vis (CH₂Cl₂): λₘₐₓ 668 nm

### 3-devinyl-20-pyridyl pyropheophorbide-a (24)

3-devinyl-20-pyridyl-methylpyropheophorbide-a (108 mg, 0.172 mmol) was added to an oven dried RBF and purged briefly with nitrogen. Ten molar HCl (22 ml) was added to dissolve under nitrogen and stirred at room temperature light protected for 8 hrs. The reaction was quenched by into iced water and extracted with chloroform followed by washes with sat. NaHCO₃ and water. It was dried over Na₂SO₄, filtered and concentrated before purifying on silica gel with gradient elution of 5-10% MeOH/CHCl₃ to obtain a dark purple solid (71% yield). R_{f} (5% MeOH/CHCl₃): 0.5. The ¹H and ¹³C NMR were broad and could not be analysed but the hydrolysis of the methyl ester was observed as the disappearance of the singlet peak at 3.58 (¹H NMR) and change in R_{f} value. MS ES (m/z) (Calc for C₃₈H₃₉N₅O₃, 613.75) Found 614.31 M⁺+H

¹H NMR (CDCl₃, 400 MHz, 25° C.) as observed for all the acid PPa derivatives this was broad leading to indistinguishable peaks making it difficult to interpret and assign. LCMS (C₁₈) retention time 9.51 min (614.3132) UV/Vis (CH₂Cl₂): λₘₐₓ 668 nm

### Methyl quaternised 5-pyridyl meso pyropheophorbide a (25)

Meso 5-pyridyl PPa (0.033 mmol) was dissolved in dry DMF (2.5 ml) under nitrogen. Methyl iodide (6.5 mmol) was added and stirred under nitrogen at room temperature for 3.5 hrs until the starting material was consumed as shown by TLC (5% MeOH/CHCl₃). Most of the solvent was removed, dry ether was added and left at 4° C. overnight before filtering to obtain a dark brown solid (74%). ¹ HNMR (CDCl₃) 9.78 (1H, d, pyridyl H), 9.52 (1H, s, δ-meso H), 9.44 (1H, s, 10-meso H), 9.10 (1H, d, pyridyl H), 9.02 (1H, d, pyridyl), 5.31 (2H, s, 13b-CH2CO), 4.33 (1H, q, 17-H), 3.9 (1H, dd, 18 CH), 3.73 (4H, m, 8aCH2 and 3a CH2CH3), 3.64 (3H, s, 12a), 3.50 (3H, s, 2a), 2.74 (3H, s, pyr Me), 1.73 (3H, t, 8b CH3CH2), 1.64 (3H, t, 3b CH3CH2), −1.38 (1H, s, NH) MS ES (m/z) (Calc for C39H42N5O3, 755.65) Found 628.33 M⁺-I; UV/Vis (DCM) λₘₐₓ 675 nm.

### Methyl quaternised 5-pyridyl meso pyropheophorbide a succinimidyl ester (26)

Methyl quartenised 5-pyridyl meso PPa (0.017 mmol) was dissolved in dry DCM (3 ml) and dry THF (1 ml). DCC (0.049 mmol) and n-hydroxysuccinimide (0.11 mmol) were added and left to stir at room temperature, light protected under nitrogen for 17 hours. Followed by TLC (Rf (10% MeOH/CHCl₃): 0.1). The solvent was removed and the solid was washed repeatedly with hexane followed by dry ether.

¹HNMR (CDCl₃) 9.53 (1H, s, δ-meso H), 9.46 (s, δ-meso H), 9.47 (1H, d, pyridyl H), 9.16 (1H, d, pyridyl H), 8.95 (1H, d, pyridyl H), 8.20 (1H, d, pyridyl H), 4.99 (2H, s, 13b-CH2CO), 4.42 (1H, q, 17-H), 4.12 (1H, dd, 18CH), 3.80 (2H, q, 8aCH2), 3.74 (2H, m, 3a CH2CH3) 3.67 (3H, s, 12a), 3.29 (3H, s, 2a), 3.20 (2H, q, 17a), 2.9 (4H, m, CH2CH2CO), 2.74 (3H, s, pyr Me) 2.75 (1H, m, 17b), 2.44 (1H, m, 17a) 2.49 (3H, s, 7a), 2.44 (1H, m, 17b), 2.19 (1H, m, 17b), 1.74 (3H, t, 8b CH3CH2), 1.64 (3H, t, 3b CH3CH2), −1.36 (1H, s, NH), MS ES (m/z) (Calc for C43H45N6O5I) Found 725.76M⁺-I

### Methyl quaternised 5-pyridyl meso methylpyropheophorbide a (27)

5-pyridyl meso MePPa (0.039 mmol) was dissolved in dry DMF (2.5 ml). MeI (7.97 mmol) was added and stirred at RT under nitrogen for 20 hours monitoring by TLC (disappearance of the starting material, 5% MeOH/CHCl3). Most of the solvent was removed and dry ether was added to obtain after cooling a brown solid (99%).

¹HNMR (CDCl3) 9.56 (1H, s, δ-meso H), 9.48 (s, δ-meso H), 9.52 (1H, d, pyridyl H), 9.35 (1H, d, pyridyl H), 8.95 (1H, d, pyridyl H), 8.20 (1H, d, pyridyl H), 4.99 (2H, s, 13b-CH2CO), 4.42 (1H, q, 17-H), 4.12 (1H, dd, 18 CH), 3.80 (2H, q, 8aCH2), 3.74 (2H, m, 3a CH2CH3) 3.67 (3H, s, 12a), 3.57 (3H, s, 17d) 3.29 (3H, s, 2a), 3.20 (2H, q, 17a), 2.74 (3H, s, pyr Me) 2.63 (1H, m, 17b), 2.44 (1H, m, 17a) 2.49 (3H, s, 7a), 2.44 (1H, m, 17b), 2.27 (1H, m, 17b), 1.73 (3H, t, 8b CH3CH2), 1.68 (3H, t, 3b CH3CH2), −1.31 (1H, s, NH) MS ES (m/z) (Calc for C40H44N5O3I 768.9) Found 642.34 M⁺-I UV/Vis (DCM) λₘₐₓ 675 nm.

### 3-devinyl-20-(4-triethyleneoxide pyridinium) pyropheophorbide-a iodide (30)

3-devinyl-20-pyridyl pyropheophorbide-a (0.060 g, 0.098 mmol) was dissolved in dry DMF (5 ml). Iodo triethylene oxide (1.48 g, 5.4 mmol) was added and the reaction was stirred at 80° C. under nitrogen light protected for four days. The reaction was monitored by TLC (silica gel, MeCN:H₂O: sat.K₂NO₃ 60:10:10) by following the consumption of the starting material.

The solvent was removed and the residual oil was redissolved in dry DCM and after cooling down filtered through cotton wool repeating several times. Redissolved in dry DCM and layered with dry ether. The brown solid was collected by centrifugation and dried over P₂O₅ in a vacuum desiccator.

MS ES (m/z) (Calc for C₄₅H₅₄IN₅O₆ 887.84) Found 760.41 M⁺-I

LCMS (C₁₈) retention time 6.84 min (760.4090) UV/Vis λₘₐₓ 677 nm

### 3-devinyl-20-(4-triethyleneoxide pyridinium)-methylpyropheophorbide-a iodide (31A)

3-devinyl-20-pyridyl-methylpyropheophorbide-a (0.100 g, 0.159 mmol) was weighed into a dry RBF under nitrogen and dissolved with dry DMF (10 ml). Iodo triethylene oxide (1.853 g, 6.76 mmol) was added and stirred at 80° C. light protected, under nitrogen for 24 hrs. The reaction was monitored by TLC (silica gel, MeCN:H₂O: sat.K₂NO₃ 60:10:10) by following the consumption of the starting material. The solvent was removed and the residual oil was washed repeatedly with dry ether, dissolved in dry DCM and filtered through cotton wool. Finally, redissolved in dry DCM and precipitated out using dry ether to obtain a brown solid which was collected by centrifugation and dried over P₂O₅ in a vacuum desiccator. R_{f} (MeCN:H₂O: sat.K₂NO₃ 60:10:10): 0.6

¹H NMR (CDCl₃, 400 MHz, 25° C.) 9.63 (1H, d, J=6.12 Hz, 20b), 9.55 (1H, s, δ-mesoH), 9.49 (1H, s, 10-mesoH), 9.38 (1H, d, J=6.24 Hz, 20b′), 8.98 (1H, dd, J6.16, 1.76 Hz 20a), 8.18 (1H, dd, J5.84, 1.28 Hz, 20a′), 5.34 (2H, dd, 2c-CH₂), 5.18 (2H, dd, 20d-CH₂), 4.47 (1H, q, 18H), 4.39 (2H, m, CH₂), 4.16 (1H, dd, 17H), 3.89 (2H, t, CH₂), 3.83 (2H, q, 8a), 3.74 (4H, m, 3a), 3.70 (4H, m,), 3.67 (3H, s, 17d), 3.57 (2H, m,), 3.56 (3H, s, 12a), 3.29 (3H, s, 2a), 3.23 (3H, s,7a), 2.76 (3H, s, 20i), 2.63 (2H, m, 17a), 2.46 (3H, s, 7a), 2.37 (2H, m, 17b), 1.70 (6H, dt, 8b 3b), 1.35 (1H, bs, NH), −1.30 (1H, s, NH).

¹³C NMR (CDCl₃, 400 MHz, 25° C.) 196.9, 173.6, 168.1, 160.8, 159.9, 153.6, 151.8, 148.9, 145.6, 145.2, 145.1, 143.9, 139.5, 139.0, 136.8, 134.1, 133.7, 131.7, 131.0, 130.5, 128.8, 106.7, 105.4, 104.6, 99.8, 71.9, 70.8, 70.4, 70.4, 70.4, 69.3, 61.9, 58.9, 52.4, 51.8, 48.5, 47.9, 34.9, 31.6, 29.7, 29.6, 21.4, 19.6, 19.4, 17.4, 17.0, 16.0, 12.1, 11.2

LCMS (C₁₈) (Calc for C₄₆H₅₆IN₅O₆ 901.87) retention time 7.38 (774.42) UV/Vis λₘₐₓ 677 nm

### 3-devinyl-20-(4-triethyleneoxide pyridinium) pyropheophorbide-a iodide (318)

Ten molar HCl (8 ml) was added to 3-devinyl-20-(4-triethyleneoxide pyridinium)-methyl pyropheophorbide-a iodide (0.0736 g, 0.082 mmol) under nitrogen and allowed to stir at room temperature light protected overnight. The reaction was monitored by TLC (silica gel, MeCN:H₂O: sat.K₂NO₃ 60:10:10) by following the consumption of the starting material. A 5 M solution of NH₄ PF₆ (˜5 ml) was added and stirred briefly. Iced water was added followed by CHCl₃ and extracted the aqueous layer several times until it was clear. The organics were further washed with water until the aqueous layer was neutral. The solvent was removed to obtain a crude product. This was purified on a preparative TLC plate (20×20 cm², 2000 p) coated with silica gel 60 using MeCN:H₂O: sat.K₂NO₃ 60:10:10 as eluent. The product was dissolved in CHCl₃ and washed with water before concentrating, redissolving in dry DCM and dry ether, collecting by centrifugation and drying over P₂O₅.

R_{f} (MeCN/H₂O/KNO₃): 0.33 LCMS (C₁₈) (Calc for C₄₆H₅₆IN₅O₆ 901.87) retention time 7.38 (774.42)

### 3-devinyl-20-(4-triethyleneoxide pyridinium) pyropheophorbide-a chloride (31C)

3-devinyl-20-(4-triethyleneoxide pyridinium) pyropheophorbide-a iodide/PF₆ (0.010 g, 0.011 mmol) was dissolved in dry MeOH (3 ml) under nitrogen. Dowex 1×8-400 (−10 mg) was added and stirred at RT light protected for 3 hrs before filtering through cotton wool and concentrating. It was redissolved in dry DCM/dry ether to obtain a solid and dried over P₂O₅ in a vacuum desiccator to obtain the final product (95%). ¹H NMR (CDCl₃, 400 MHz, 25° C.) again this is quite broad and difficult to assign 10.27 (1H, s, 20b), 9.52 (1H, s, meso 5H), 9.37 (1H, s, meso 10H), 9.18 (2H, s, 20a,20a′), 8.02 (1H, s, 20b′), 5.48 (1H, m, 13b), 5.21 (1H, m, 13b′), 4.71 (1H, m, 18H), 4.30 (4H, m, CH₂), 4.19 (1H, m, 17H), 3.78 (2H, m, CH₂), 3.74 (2H, q, 3a), 3.66 (2H, m, 8a), 3.61 (3H, s, 12a), 3.49 (2H, m, 17a), 3.28 (3H, s, 2a), 3.16 (3H, s, 7a) 2.61 (6H, s, 20i), 2.32 (3H, m,17b), 1.73 (3H, t, 8b), 1.53 (3H, t, 3b), −1.51 (1H, bs, NH).

¹³C NMR (CDCl₃, 400 MHz, 25° C.) 134.1, 132.6, 132.2, 131.5, 128.8, 128.5, 126, 106.4, 104.9, 99.2, 71.8, 70.6, 70.3, 70.3, 69.5, 61.2, 58.6, 54.1, 47.8, 46.0, 42, 40.9, 32.5, 29.7, 28, 22.7, 20.7, 19.5, 19.3, 17.3, 16.8, 15.3, 14.1, 12.1, 11.2.

LCMS (C₁₈) (Calc for C₄₅H₅₄F₆N₅O₆ 905.90) retention time 6.80 (760.41 M⁺-PF₆) UV/Vis (DCM) λₘₐₓ 676 nm

### EXAMPLE 2

### Conjugation of the Compounds of the Invention to Carrier Molecules and Biological Analysis of the New Photoimmunoconjugates

### Biological Materials and Methods

### Cell Culture:

Human tumour cell lines (SKOV3) were grown in 75 cm³ flasks, washed with PBS (2×25 ml) and incubated with trypsin (10×) (2 ml per 150 cm³ flask) for 5-7 min. phenol red free DMEM (10% FBS, 1% Penicillin/streptomycin) was added (10 ml) and the cells were re-suspended by pipetting. The cells were transferred to a falcon tube and centrifuged for 2 mins, 2000 rpm, 37° C. The supernatant was discarded and the pellet was re suspended in 5 ml of phenol red free DMEM (10% FBS, 1% Penicillin/streptomycin). The cells were counted using a haemocytometer, diluted accordingly and plated 200 μl per well.

Plated as follows:

SKOV-3 3000 cells/well, KBs 2000 cells/well, SKBr3 5000 cells/well

C6.5 scFv was obtained from Prof. J. Marks (University of California, San Francisco) in pUC119 and expressed in XL1 blue cells. The C6.5 scFv was engineered to remove a lysine-100 in the antibody binding site. This was to reduce the possibility of forming PICs of reduced immunoreactivity.

Purified protein was either concentrated to 1 mg/ml protein using 25 ml spin concentrators and stored in 10% glycerol at −80° C., or used for couplings straight after purification without concentrating.

Synthesis of C6scFv-Photosensitizer Photo-Immunoconjugates (PICs)

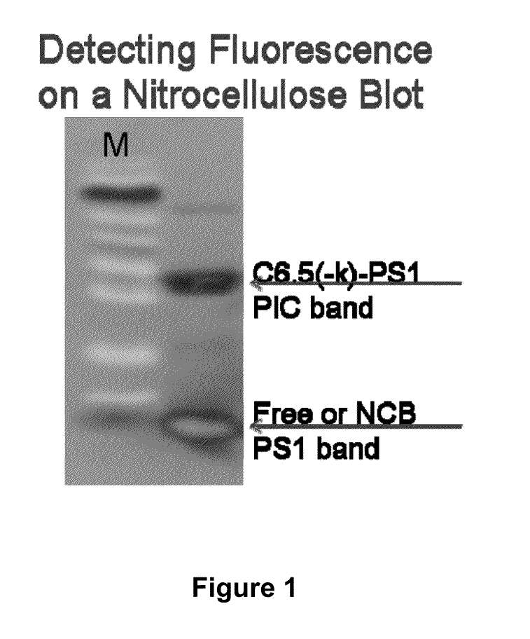

The PPaPEG succinimidyl ester was re-suspended in 100% DMSO and added (maintaining the total DMSO content at 2%) at a concentration of 592 μM to 37 μM of C6scFv in PBS containing 6% acetonitrile and with continuous stirring at room temperature for 1 h. The photoimmunoconjugates (PICs) were then dialyzed against PBS/2% DMSO with two buffer changes. The photoimmunoconjugates (PICs) were then dialyzed against PBS with two buffer changes. Sodium dodecyl sulphate polyacrylamide gel electrophoresis (SDS-PAGE) analyses was carried and stained with coomassie blue. Nonstained gels were transferred using a semidry blotting apparatus (Biorad) onto nitrocellulose and gently dried. Fluorescence was visualized by exciting the PPaPEG with a UV light using Fuji LAS3000. Using the manufacturers' software, densitometry was used to calculate the relative intensities/band sizes. This ratio was used to correct for noncovalent binding. Under UV illumination (see FIG. 1), free and conjugated PPaPEG fluoresced confirming covalent coupling and indicated that about 30% of PPaPEG was non-covalently associated with the protein.

Further details of appropriate conjugation conditions may be found in WO 2007/042775 and in Bhatti et al (2008) Int J Cancer Mar 1; 122(5):1155-63.

For PPa, coupling ratios have varied from 30-50%. The importance of improving Photosensitiser (PS) solubility and minimising aggregation between PS in the conjugation conditions is allowing us to carry out conjugations at significantly higher PS concentrations than we could using a inherently hydrophobic PS like PPa. As an example we are aiming to conjugate at a protein concentration of 5 mg/ml, this would require a PS concentration of approx 130 mg/ml in essentially PBS. Using a hydrophobic PS like PPa, allowed us to carryout conjugations at a protein concentration of 250 pg/ml. So by improving PS solubility and minimising aggregation we can achieve a 200 fold increase in the amount we can conjugate, leading to higher concentration photoimmunoconjugates.

### Spectroscopic Measurements

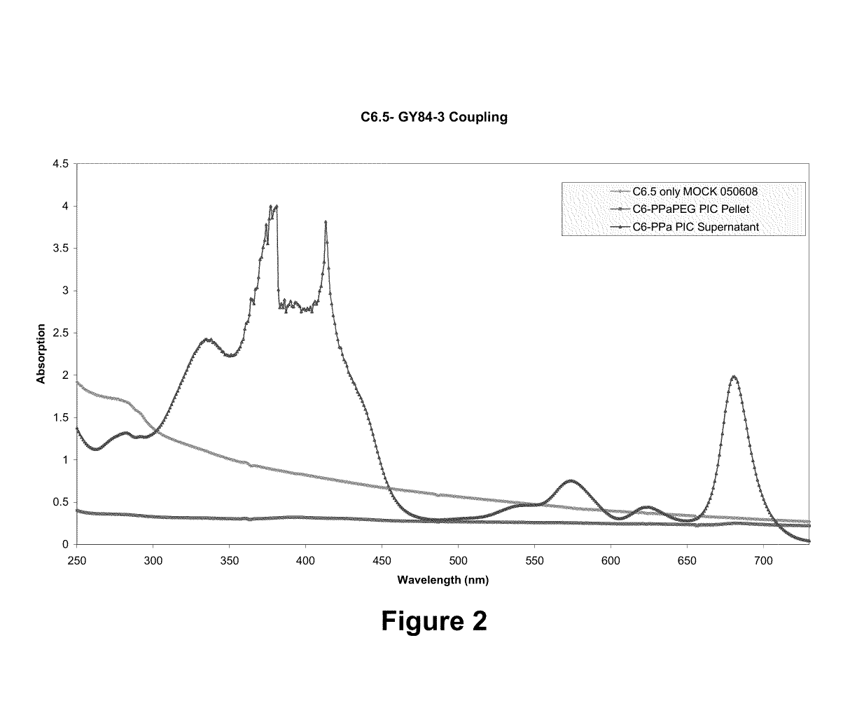

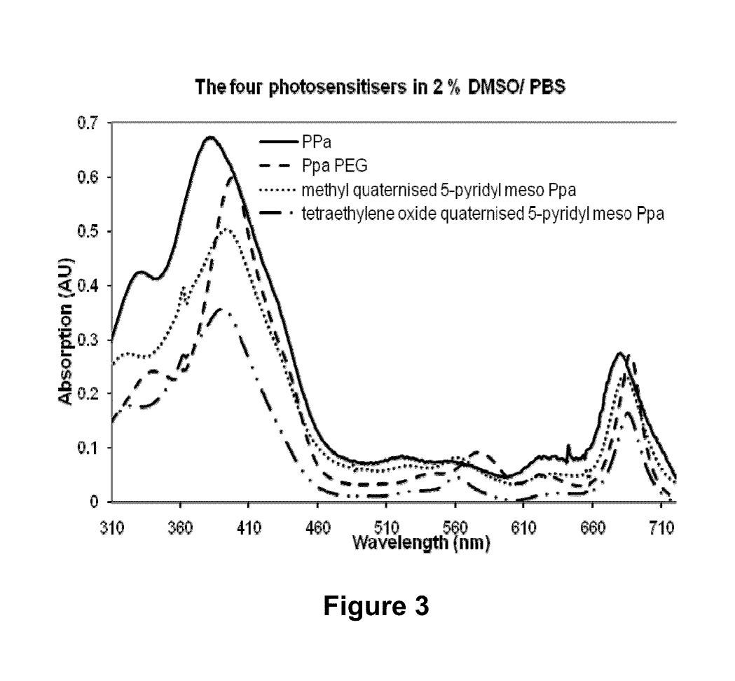

The absorbance profile of the free photosensitizer (dissolved in 2% dimethyl sulphoxide-DMSO/PBS) and photosensitizer coupled to the scFv (dissolved in PBS/2% DMSO) was determined on a Hewlett Packard UV-Visible spectrophotometer (FIGS. 2 and 3). The number of PPaPEG molecules attached to the scFv was determined using the absorbance at 410 nm and 670 nm and compared to a standard curve of PPaPEG. The absorbance profile of C6PPaPEG conjugate (FIG. 2), shows the characteristic peaks around 400 nm (Soret band), minor peaks between 500 and 630 nm and an intense absorption around 670 nm, which is characteristic of chlorins (Q bands). The peaks have broadened slightly with a 3-5 nm red-shifted 670 nm peak compared to free PS (data not shown) but are all sharp indicating a disaggregated state for the PS. This peak at around 670 nm was used to determine the PPaPEG:scFv ratio and gave an effective ratio range of 5-10:1 PS:scFV after correction for 30% (determined by densitometry) of noncovalent binding.

In Vitro Cytotoxicity of C6scFV-PPaPEG PIC

Cells were trypsinized and seeded at 3×10³ cells/well into 96-well plates and incubated overnight at 37° C. and 5% CO₂. The next day, the cells were washed once in PBS and 50 μl of the PICs (appropriately diluted) or free photosensitiser were added to the appropriate wells under subdued lighting. PBS was added to control wells. After 30 min incubation in the dark at 37° C., 5% CO₂, cells were washed 3 times with PBS and 50 μl of PBS was added to each well. Wells were exposed to light from a 2 W (680 nm; HPD Inc, New Jersey, USA) at a dose over 4 wells of 0.6 W for 10 sec. (control wells had either scFv-PPaPEG or free PPaPEG added and no exposure to light, or PBS added and exposure to light. Cells that had no scFv-PS or PS added and no exposure to light were included as overall controls). Cells were incubated in the dark at 37° C., 5% CO₂ for 48 hr after which time, a cell titer assay was performed according to the manufacturers instructions. The Promega Cell Titre-96™ system was used which involves the conversion by live cells of a tetrazolium compound (MTS) into a formazan dye which is measurable by its absorbance at 492 nm. For C6.5 containing PICs, SKOV-3 cells were used as the antigen (HER2) positive cell line and KB cells used as the antigen negative cell line.

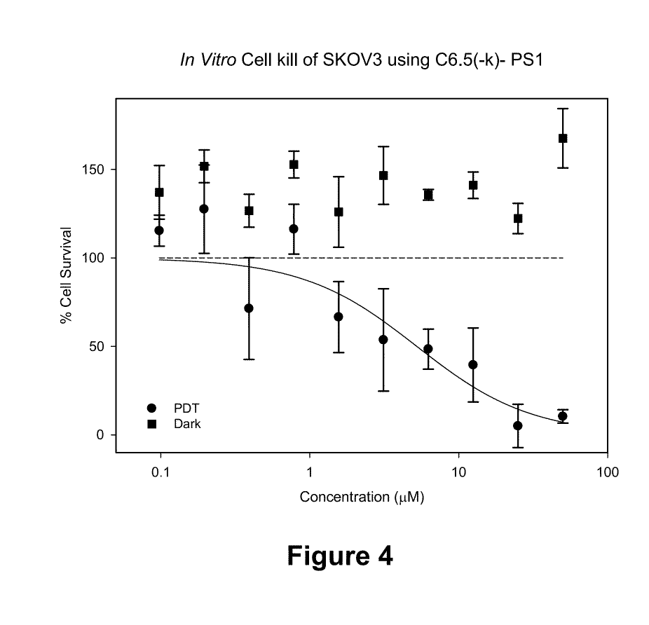

The C6PPaPEG PIC killed its receptor expressing cell line (see FIG. 4) and spared the receptor negative cell line (data not shown).

### In Vivo Experiments (Biodistribution, Pharmacokinetics and Therapy)

### Biodistribution Experiments

0.1 mg of photosensitiser was dissolved in 0.5 ml PBS. Radiolabelled Iodine (Na¹²⁵I, ICN chemicals) was added to 0.1 ml of PBS in Iodogen-coated tubes (Pierce chemicals) and allowed to activate. The activated iodine-125 was transferred to the photosensitiser and allowed to react for 5-10 minutes at room temperature. The radiolabelled photosensitiser was separated from free iodine on a mini silica gel column eluting with 100% PBS to remove the iodinating agent followed by a gradual switch from 10% MeOH/PBS to 100% MeOH with the radiolabelled photosenstiser eluting with 10% MeOH/chloroform. The radiolabelled solutions of the photosensitiser was allowed to air-dry and re-suspended in PBS containing 2% DMSO.

Female BALB/c nude mice (aged 6-8 weeks old) were implanted with 10⁷ SKOV3 tumour cells mixed in 0.1 ml of ice-cold matrigel subcutaneously and the tumours were allowed to grow for 3-6 weeks as xenografts. Mice were maintained in IVC cages (individually-vented cages) in a clean room. Samples were injected in a volume of 0.1 ml, intravenously via the lateral tail vein and the mice were maintained in low light with full food and water (irradiated). At various time points (1-24 hr), mice were culled by cardiac puncture under terminal anesthesia and blood and tissues collected. All the tissues were counted for radioactivity by gamma counting and weighed.

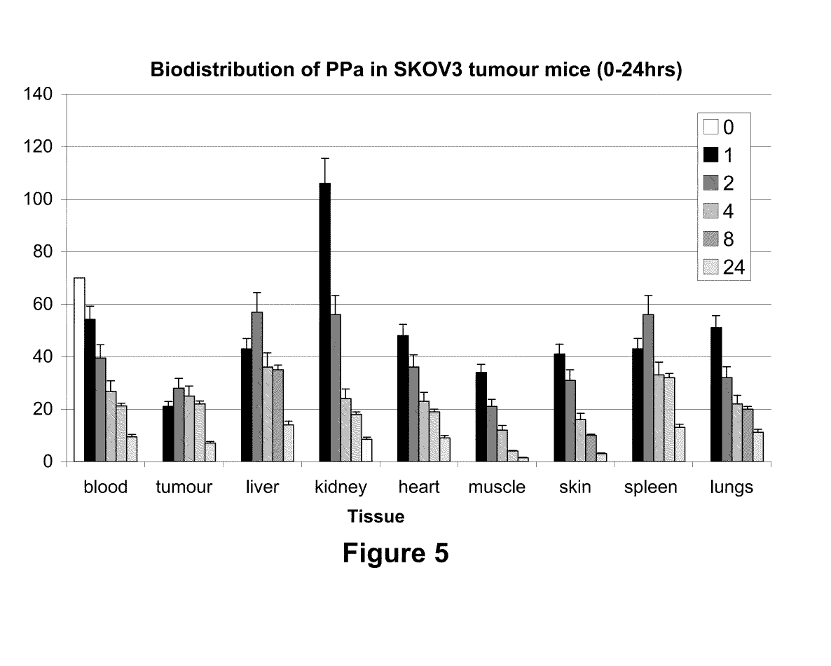

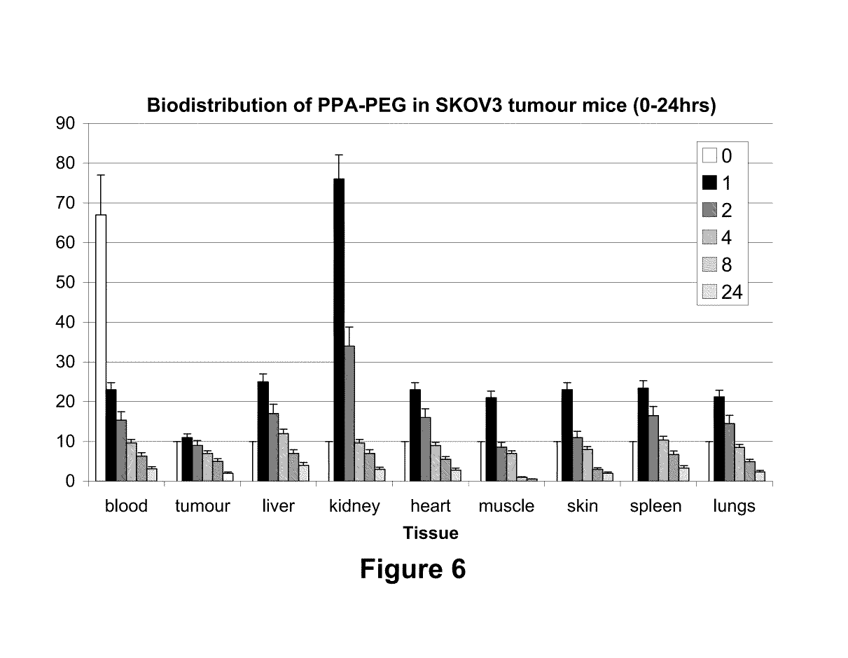

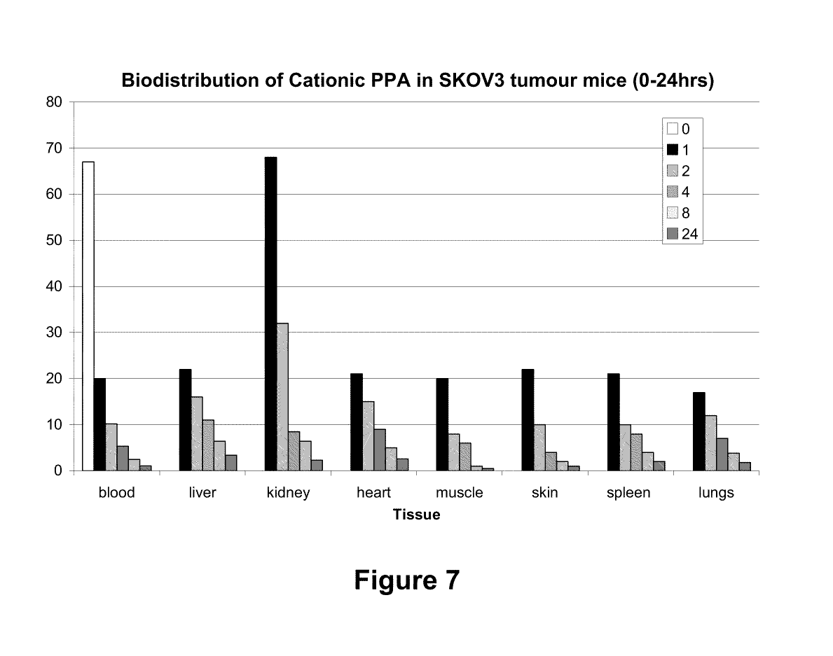

The results are shown as a percentage of radioactive material injected per gram of tissue. The biodistribution of PPa is shown in FIG. 5. The biodistribution of PPa-PEG is shown in FIG. 6. The biodistribution of Cationic PPa is shown in FIG. 7.

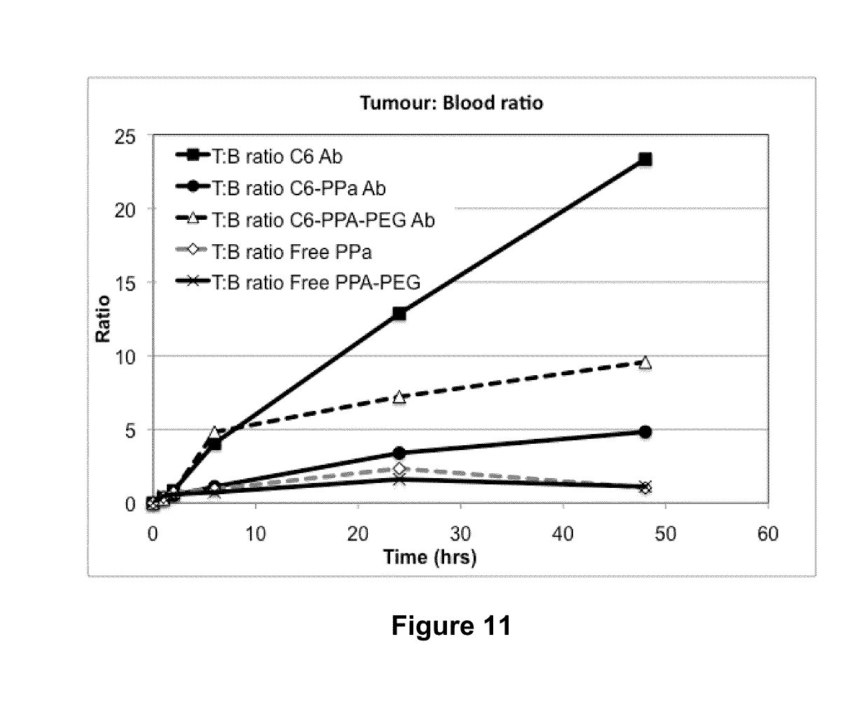

PPa is hydrophobic and resides in the blood for a long time. This leads to high levels in all major tissues, which accounts for the skin photosensitivity for many commercial photosensitisers. There is no significant tumour localization with any of these PS (see Tumour:blood ratios plot in FIG. 11). The more soluble PS clear more rapidly and have a lower overall level in all the tissues. For the soluble PS, there is no specific localization to any tissue.

### Blood Clearances of All 3 Photosensitisers

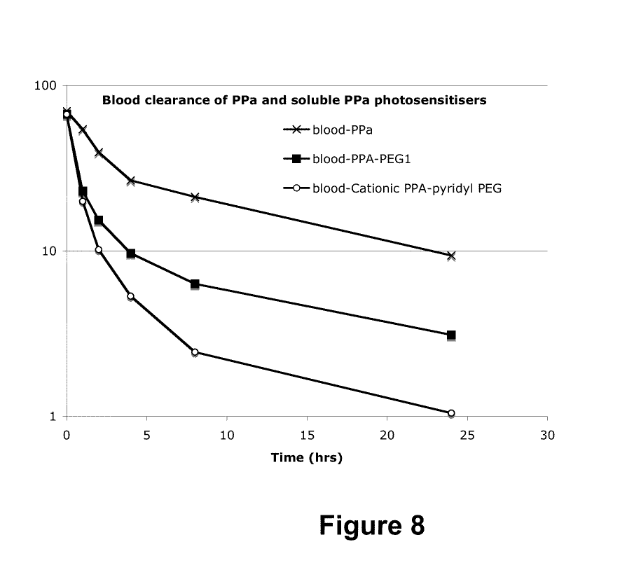

The blood clearance of all three photosensitisers is plotted in FIG. 8. The cationic PPa PS clears more rapidly than the PPa-PEG which are both faster than PPa.

These results indicate that the design features of the new photosensitisers of the present invention lead to more solubility and faster clearance, which is desirable.

### Blood Clearances of All C6-PPA-PEG Photosensitisers Compared to C6-PPA

The PS(PPa and PPa-PEG) were chemically coupled to C6 scFv, radiolabelled with iodogen as before, but dialysed against 3×5 L of PBS to remove the free iodine and injected into BALB/c nude mice with SKOV3 tumours as above. Tissues were dissected and counted as above.

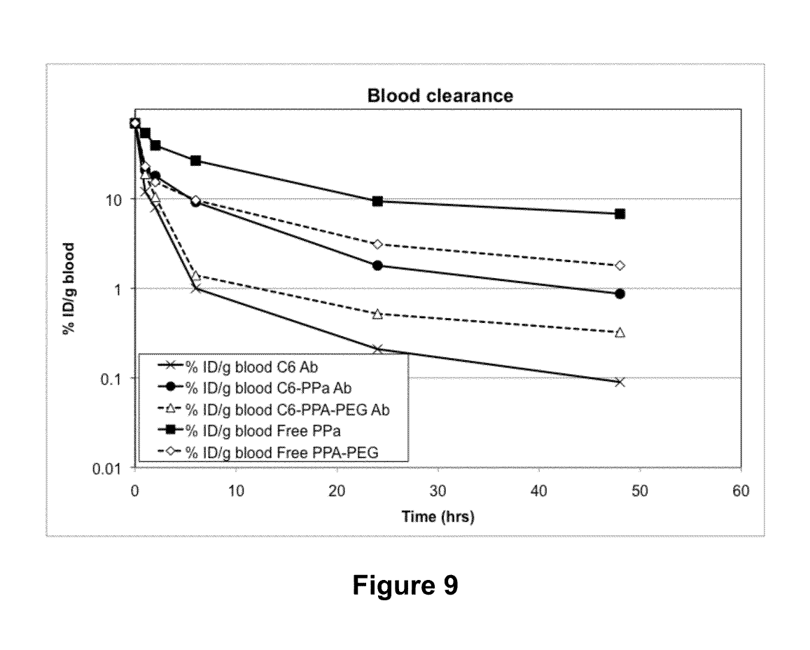

The blood clearance was monitored and the results are shown in FIG. 9. C6 scFv has a very fast blood clearance. PPa has a very slow blood clearance. The C6-PPa blood clearance was in between that of the 2 components as was the C6-PPa-PEG.

This shows that the particular PS that is conjugated modulates the rate of clearance of the scFv and that the more soluble/hydrophilic the PS (i.e. PPa-PEG) the more rapidly the conjugate clears. This suggests that the more soluble scFv-PS conjugates, as provided by the present invention, could have lower toxicity and skin photosensitivity and better tissue:blood ratios.

### Tumour Uptake

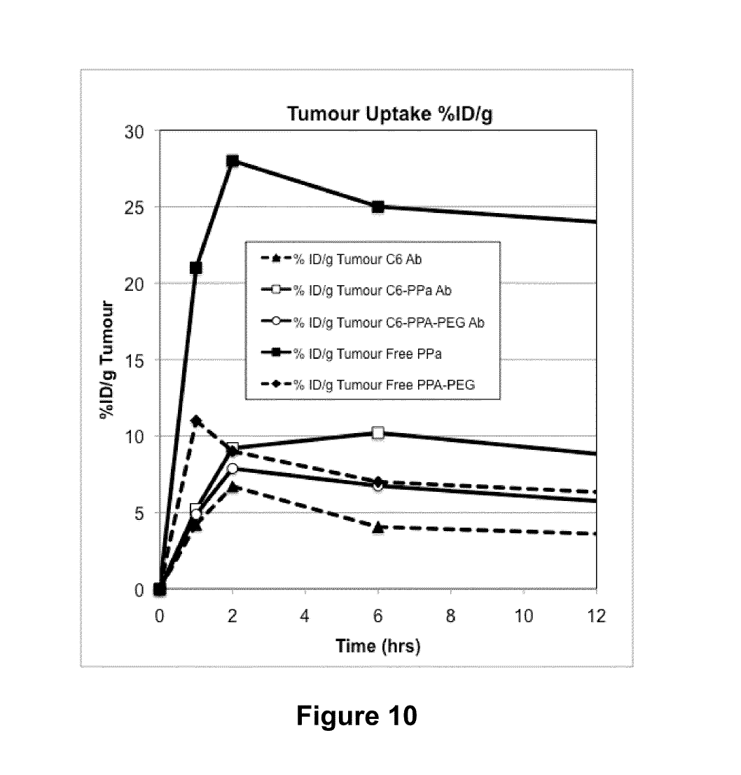

The tumours were counted for radioactivity and expressed as % of the injected dose/g of tumour tissue (FIG. 10).

PPa has a high tumour (and high normal tissue) uptake due to its long blood half-life. The PPa-PEG accumulates in tumours at about ⅓ the level of PPa. The C6 scFv, due to its rapid clearance, accumulates at lower levels, peaking at 2 hrs. The two PS conjugates accumulate in the tumours, with more being present for the C6-PPa conjugate than the C6-PPa-PEG due to the faster clearance of the latter.

This data again suggests that the PIC of the invention will have lower side effects but retain tumour localizing properties. The tumour:blood ratios are also better (see FIG. 11).

### Tumour:Blood Ratios of PS and PICs

The percentage PS in the tumour was divided by the percentage in the blood (gram for gram) to give a targeting or tumour:blood ratio (the higher the better). This plotted in FIG. 11. A high tumour:blood ratio means that there is more in the tumour compared to the blood (and other tissues). This is a function of tumour uptake and blood clearance (see FIGS. 9 and 10).

The C6 scFv has the highest ratio due to its binding and fastest clearance. For example, the ratio is 12:1 at 24 hrs. The ratio is increasing over time due to retention in the tumour but clearance from the blood. The free PS have poor ratios (around 1-2) due to non-targeting. The C6-PPa has a ratio of 3:1 at 24 hr rising to 5:1 at 48 hrs. This ratio is improved for the C6-PPa-PEG PIC due to the faster clearance. This ratio is 7:1 at 24 h rising to almost 10:1 at 48 h.

These results suggest that more soluble PS lead to more soluble PICs which have better specificity and targeting than free PS. However, there is less PIC (and hence sensitiser) in the tumour than if non-targeted.

PPa-PEG Only Therapy (Without scFv Conjugation)

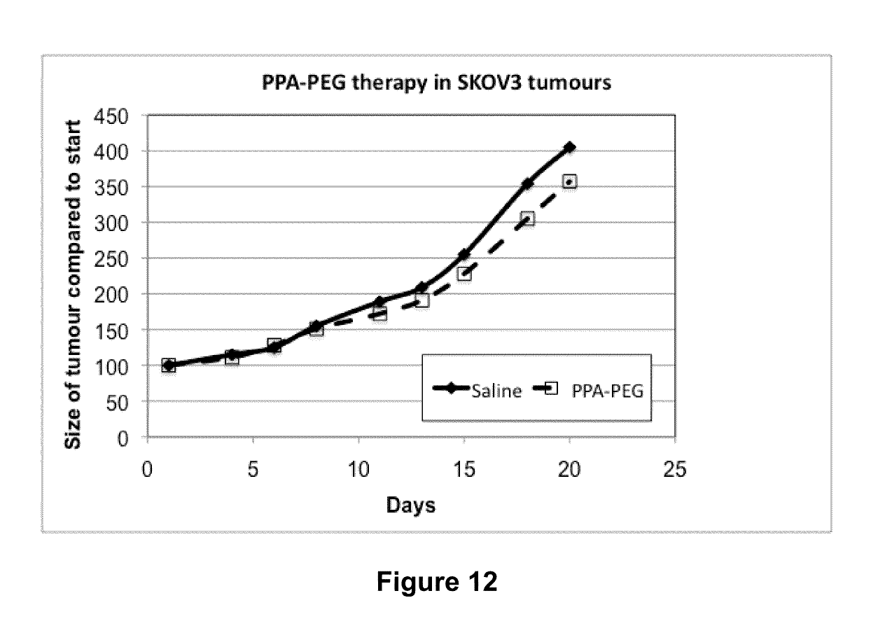

Tumours were set up as described above and injected (iv into the mouse tail vein) with 0.2 ml of free PPa-PEG1 (0.1 mg=33 micromolar concentration) PS on day 1 and day 4. Based on the tumour uptake (see FIG. 10), an HPD laser was used to illuminate the tumours for 20 minutes at 0.5 W after 4 hrs. The mice were anaesthetized beforehand. Laser treatment followed each drug cycle. The tumours were followed for 3 weeks and the % increase in tumour size was plotted (day 0=100%) (see FIG. 12).

PPa-PEG caused no significant tumour regression compared to untreated (Saline) controls. This is probably due to the rapid clearance of the PS.

### C6 PPa-PEG Therapy

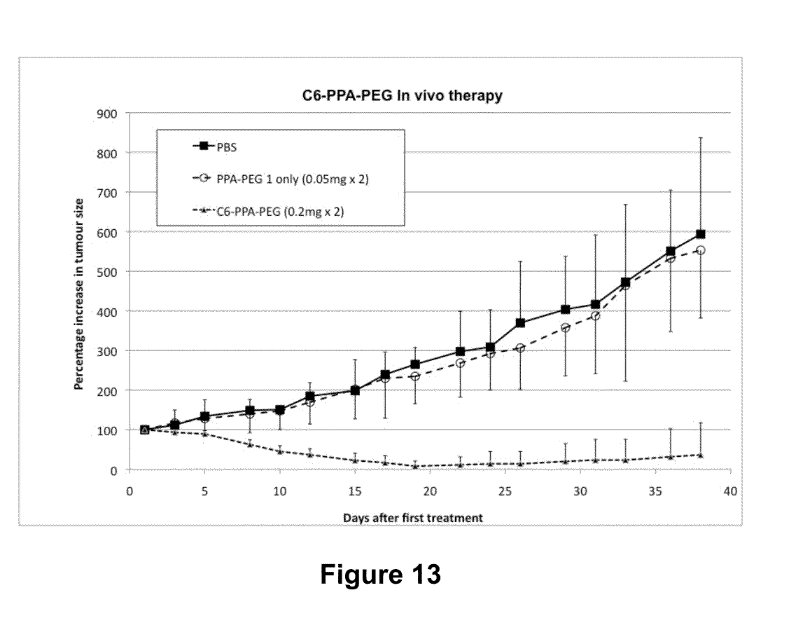

Tumours (groups of 6 mice) were set up as described above and injected (iv into the mouse tail vein) with 0.2 ml of C6-PPa-PEG PIC or free PPa-PEG1 PS (1 mg/ml PIC=33 micomolar concentration scFv or 330 micromolar PPa-PEG) on day 1 and day 4. The free PPa-PEG PS was at the same concentration (0.5 mg/ml=375 micromolar). Based on the tumour uptake (see FIG. 10), a HPD laser was used to illuminate the tumours for 20 minutes at 0.5 W after 4 hrs. The mice were anaesthetized beforehand. Laser treatment followed each drug cycle. The tumours were followed for 6 weeks and the % increase in tumour size was plotted (day 0=100%) (see FIG. 13).

PPa-PEG caused no significant tumour regression compared to untreated (Saline) controls. This is probably due to the rapid clearance of the PS. However, the HER-2 targeted PPa-PEG caused significant tumour regression (p<0.001), with 5/6 of the mice successfully being cured of their tumours.

These results demonstrate the therapeutic utility of the C6-PPa-PEG PIC and indicate that higher doses of PIC can be administered in vivo which could lead to a better therapeutic outcome.

### EXAMPLE 3

### Further Derivatives of PPa

### Overview

A number of further derivatives of pyropheophorbide-a are capable of synthesis by suitable manipulation of functional groups around the periphery of the macrocycle, giving a series of new derivatives with enhanced solubility in PBS and further reduction in self aggregation. All of these derivatives contain a group in which a bioconjugatable tether like hexynoic acid can be attached using a metal catalysed cross-coupling reaction (Sonogashira Coupling).

Although the 17-propionic side chain of PPa projects above the macrocyclic plane (as discussed earlier) minimising self aggregation, the other side still presents a large hydrophobic face to the surroundings. To overcome this, we introduce both TEG-chains and charged groups on to the C-3 carbon atom. We expect such groups to swing away from the side containing the 17-propionic acid side chain and reside over the unprotected side of PPa.

In one example of modification (scheme 10), the vinyl group of MPPa (5) was converted to the aldehyde, giving methylpyropheophorbide-d (32) in good yields as a fine-brown powder. The aldehyde was oxidised by using a published procedure (ref 102) using sodium chlorite in the presence of a chlorine-atom scavenger, 2-methyl-2-butene, giving the corresponding carboxylic acid (33) in 40-50% yield. The benzyl ether unit with short tri(ethylene glycol) monomethyl ether (TEG) chains was then attached through esterification of the 3-carboxylic acid, giving (34), which was then brominated using pyridinium perbromide to give derivative (35)

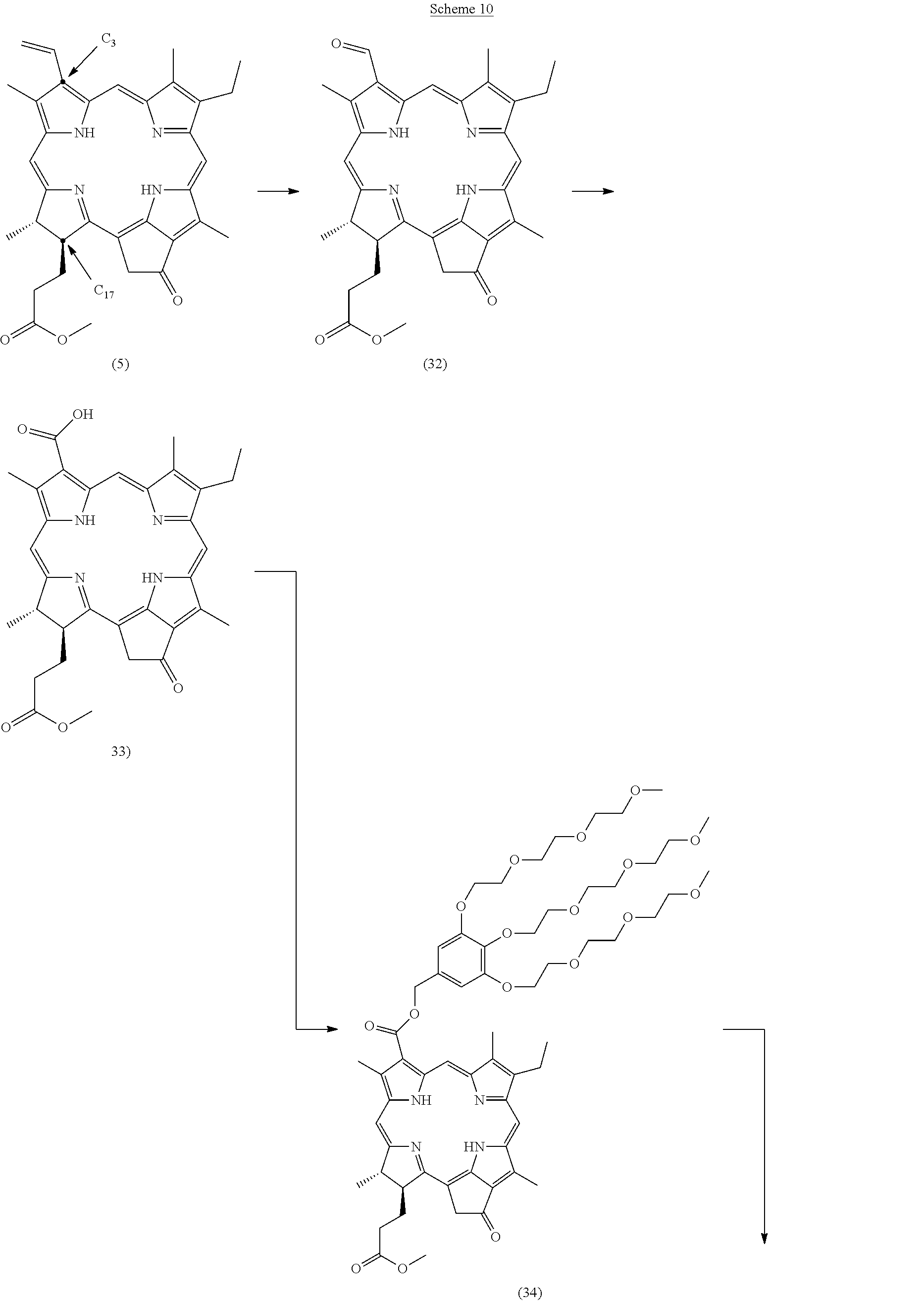

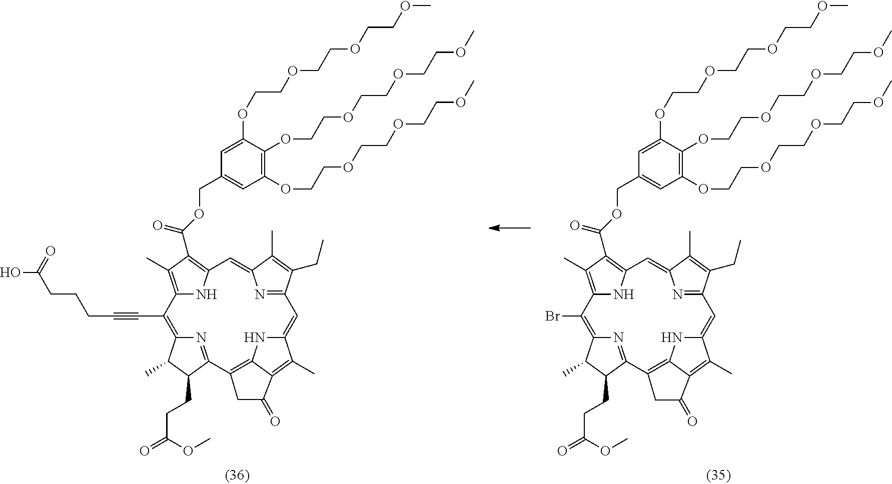 [SMILES: CCC1=C(C)c2cc3[nH]c(c(C)c3C(=O)OCc3cc(OCCOCCOCCOC)c(OCCOCCOCCOC)c(OCCOCCOCCOC)c3)c(C#CCCCC(=O)O)c3nc(c4c5[nH]c(cc1n2)c(C)c5C(=O)C4)[C@@H](CCC(=O)OC)[C@@H]3C ; CCC1=C(C)c2cc3[nH]c(c(C)c3C(=O)OCc3cc(OCCOCCOCCOC)c(OCCOCCOCCOC)c(OCCOCCOCCOC)c3)c(Br)c3nc(c4c5[nH]c(cc1n2)c(C)c5C(=O)C4)[C@@H](CCC(=O)OC)[C@@H]3C]

Hydrolysis of the 17-propionic ester side chain in (33) with strong acid (HCl, H₂SO₄) gave the di-carboxylic acid derivative (37), scheme 11. By esterifying both carboxylic acids with the TEG chains gave the highly aqueous soluble derivative (38) in which both planes of the macrocycle are protected and prevented from self-association. Bromination gives the meso 5-bromo derivative (39), again allowing the introduction of the bioconjugatable tether.

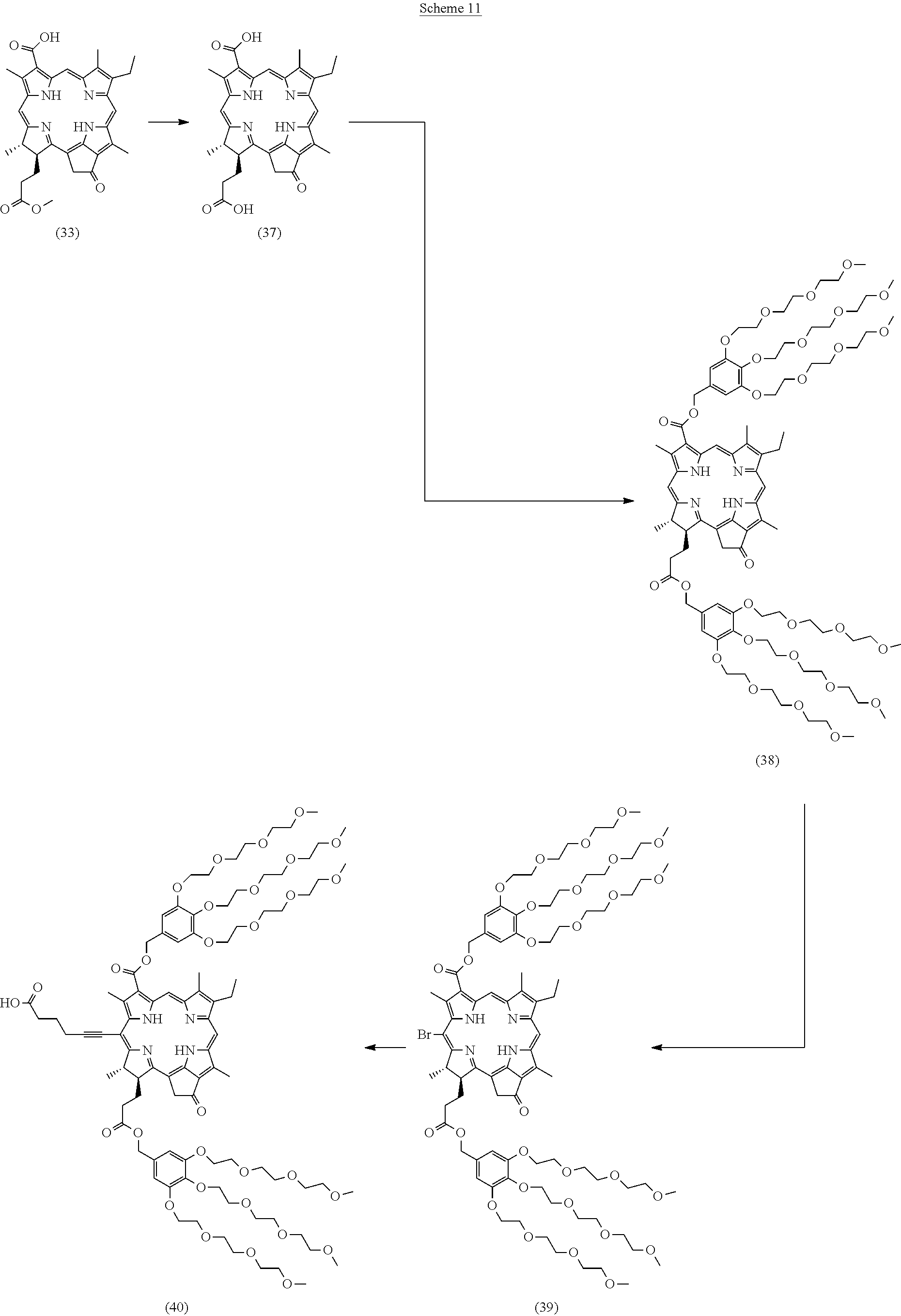

Further attempts at introducing ‘swallow-tail’ like solubilising groups, which would extend over the planes of the macrocycle involved converting the 3-aldehyde group of methyl pyropheophorbide-d (32) in to an ethynyl group, (scheme 12). This was achieved in one step using the Bestmann-Ohira reagent in the presence of caesium carbonate and dry methanol (ref 103) in modest yields.

The reaction was followed spectroscopically, monitoring the disappearance of the 694 nm peak of the 3-formyl derivative (32) and the appearance of a peak at 677 nm, corresponding to the 3-ethynylated derivative (41), isolated as a purple powder after chromatography. The presence of the C3-ethynyl group in (41) enabled us to carry out a copper-free Sonogashira coupling with excess 2-ethoxy(triethyleneoxy)-iodobenzene (42), giving phenyl derivative (43). Hydrolysis of the 17-propionate ester chain to the corresponding acid was achieved using LiOH/THF/MeOH, giving a conjugatable handle.

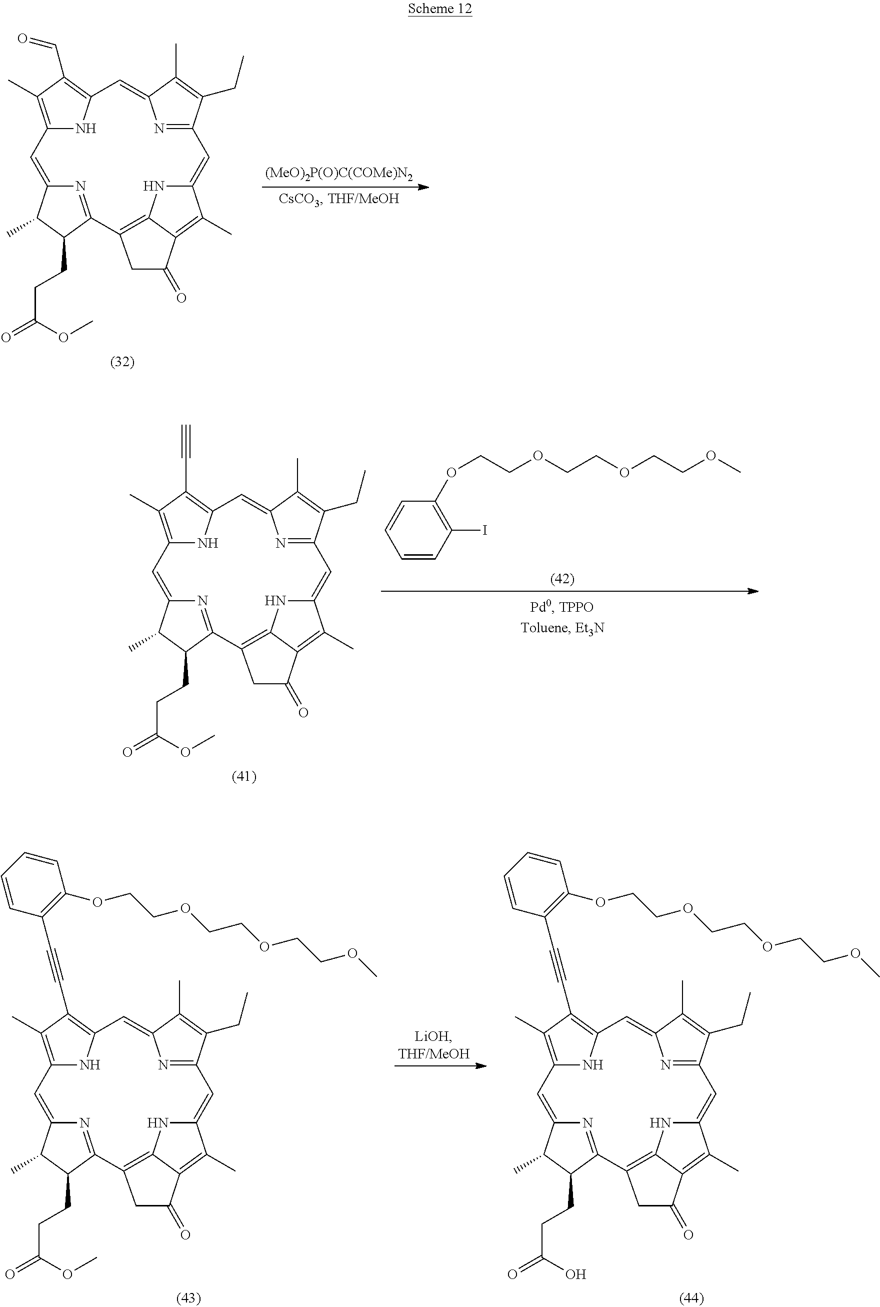

The presence of a C3-ethynyl group opens up the possibility of carrying out 1,3-diploar cyclo-additions with azido compounds (‘Click’ chemistry), and although this has already been demonstrated with compound (41) (ref 103).

We decided to try and attach either an alkyne or an azide functionality to the 17-propionic acid side chain of meso-PPa (17), scheme 13. Meso-PPa (17) was esterified with propargylamine using the two-step procedure developed by us, giving the amide (45) in high yields. This involves preforming the N-hydroxysuccinimide derivative of the acid in situ by reacting the acid with NHS in the presence of a dehydrating agent such as DCC or DIC. The reaction was followed by TLC and once all the starting acid (17) was consumed and a spot with a higher R_{f} had appeared, the propargylamine was added in one go and stirring at room temperature continued for a further 5 hr, when the reaction was complete as judged by TLC.

The propargylamide derivative (45) was isolated as a purple powder after chromatography and brominated with pyridinium perbromide to give the meso 5-bromo derivative (46). This was metallated with zinc to give (47) using zinc acetate in refluxing chloroform/methanol. This is necessary to prevent copper insertion into the macrocycle, the cycloaddition reaction between an azide and an alkyne is normally carried out in the presence of copper sulphate and sodium ascorbate.

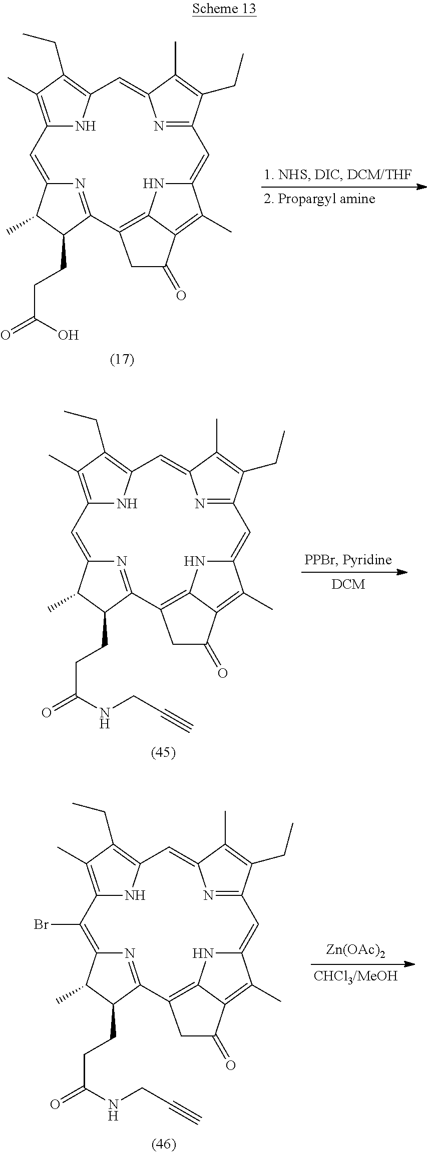 [SMILES: CCC1=C(C)c2cc3[nH]c(cc4nc(c5c6[nH]c(cc1n2)c(C)c6C(=O)C5)[C@@H](CCC(=O)O)[C@@H]4C)c(C)c3CC ; C#CCNC(=O)CC[C@@H]1c2nc(cc3[nH]c(cc4nc(cc5[nH]c6c2CC(=O)c6c5C)C(CC)=C4C)c(CC)c3C)[C@H]1C ; C#CCNC(=O)CC[C@@H]1c2nc(c(Br)c3[nH]c(cc4nc(cc5[nH]c6c2CC(=O)c6c5C)C(CC)=C4C)c(CC)c3C)[C@H]1C ; "Zn(OAc)2"]

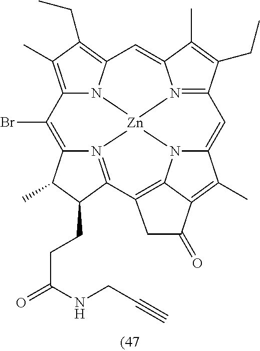

We also looked at extending the absorption profile of our pyropheophorbide derivatives to longer wavelengths (700-800 nm) by converting them to the corresponding bacteriochlorins, enabling us achieve deeper penetration into tumours thus treating larger tumour masses.

Pandey et. al. (ref 104) have demonstrated that both pheophorbide a and pyropheophorbide a react with osmium tetraoxide to produce the corresponding vic-dihydroxybacteriochlorin in good yields. The meso 5-bromo triPEG derivative (9) was converted to the corresponding bacteriochlorin derivative (47) by reacting with OsO₄ in dry DCM containing a small amount of pyridine. The reaction was stirred at room temperature for 12 h and was monitored by UV/Vis spectroscopy, during which dramatic changes to both the Soret band (a blue shift from 415 nm to 361 nm) and the furthest Q-band (a red shift from 668 nm to 715 nm) were observed, both characteristic of bacteriochlorins. Sonogashira coupling with the hexynoic acid will give us a water soluble bioconjugatable bacteriochlorin derivative.

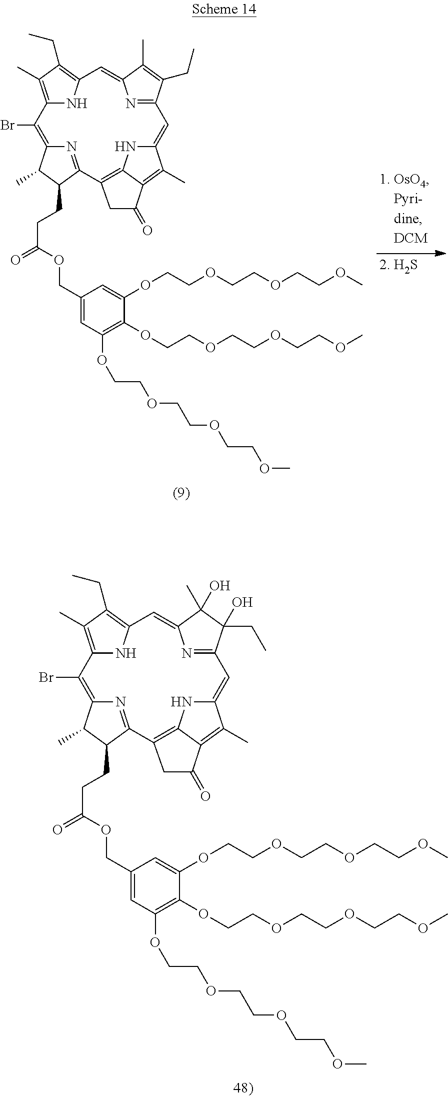 [SMILES: CCC1=C(C)c2cc3[nH]c(c(C)c3CC)c(Br)c3nc(c4c5[nH]c(cc1n2)c(C)c5C(=O)C4)[C@@H](CCC(=O)OCc1cc(OCCOCCOCCOC)c(OCCOCCOCCOC)c(OCCOCCOCCOC)c1)[C@@H]3C ; CCc1c(C)c2[nH]c1cc1nc(cc3[nH]c4c(c5nc(c2Br)[C@@H](C)[C@@H]5CCC(=O)OCc2cc(OCCOCCOCCOC)c(OCCOCCOCCOC)c(OCCOCCOCCOC)c2)CC(=O)c4c3C)C(O)(CC)C1(C)O]

The utility of our approach and the development of intermediates such as the meso 5-bromo derivatives allowed us to easily develop photosensitisers for site-specific coupling onto the thiol groups of cysteine. Cysteine residues can represent an attractive bioconjugation target because, unlike lysine residues, cysteines on antibodies are remote from the binding site.

One of the most common and important reactive groups for coupling with thiols are maleimides, which undergo an alkylation reaction with thiols to form stable thioether bonds. A substituted linear alkyne derivative containing a maleimide group (54) was synthesised from 5-aminohexyne (52) by reacting with maleic anhydride to give the intermediate (53) which upon treatment with sodium acetate and acetic anhydride afforded the N-pentynemaleimide (54).

By attaching compound (54) to various meso 5-brominated derivates through the Sonogashira coupling, we are able to carry out conjugations on to cysteine residues with our photosensitisers. Compound (52) was prepared according to literature methods (ref 105)

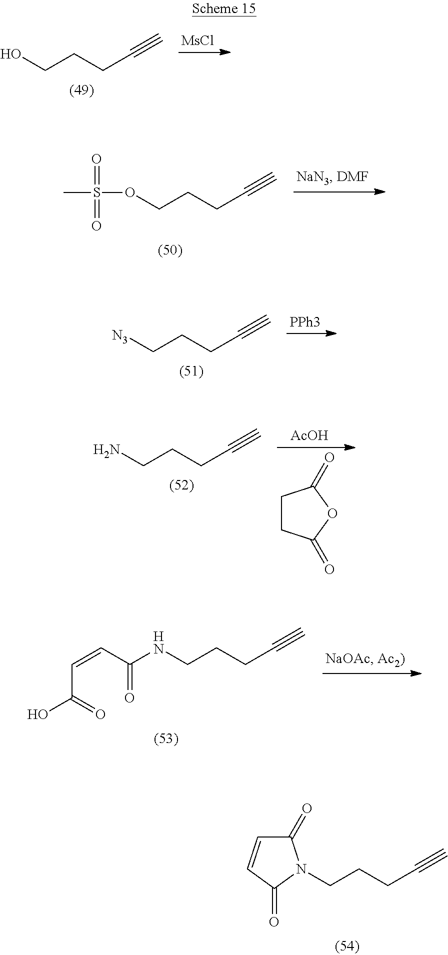

The 5-azidopentyne (51) is a useful intermediate as it can also be coupled onto the meso 5-bromo position allowing groups to be attached through cyclo-addition reactions with azides.

### Synthetic Routes and Methods

The manipulation of air and/or water sensitive compounds was carried out using standard Schlenk techniques. DCM and triethylamine were dried by distilling from CaH₂ and dry THF was obtained by distillation from sodium/benzophenone. All other reagents were used as supplied by commercial agents unless stated otherwise.

Analytical thin layer chromatography (TLC) was carried out on Merck glass backed silica gel 60 GF₂₅₄ plates or aluminium backed aluminium oxide (neutral) and visualisation when required was achieved using UV light or in some cases a chemical staining agent was used. Column chromatography was carried out on silica gel 60 or aluminium oxide(neutral or basic) deactivated with 5% water (referred to as Brockmann grade III) using a positive pressure of air. Where mixtures of solvents were used ratios reported are by volume.

NMR spectra were recorded at ambient probe temperature using a Bruker DPX400 (400 MHz). Chemical shifts are quoted as parts per million (ppm) with CDCl₃ as internal standard (for ¹H NMR, 7.26 ppm) and coupling constants (J) are quoted in Hertz (Hz). UV/Vis spectra were recorded on a Hewlett Packard 8450 diode array spectrometer. Mass spectra were carried out using a number of techniques and only molecular ions and major peaks are reported.

### Pyropheophorbide-d (32)

This was prepared following a combination of literature procedures but primarily [Chem. Eur. J. 2008, 14(26), 7791-807] with minor modifications. To a solution methyl pyropheophorbide a(5) (0.5 g, 0.9 mmol) in anhydrous THF (120 ml), glacial acetic acid (1.5 ml) and water (1.5 ml) were added followed by osmium tetroxide (3 mg, a few small crystals). The reaction mixture was stirred at room temperature for 30 min. after which TLC [silica gel: 5% MeOH/DCM] showed the formation of the dihydroxylated intermediate, although this is not clear. After stirring for an additional 30 min. a satd. solution of sodium metaperiodate (25 ml) in water was slowly added using a pressure equalising addition funnel at an approx. Flow rate of 10 ml/h. Subsequently a further portion of sodium metaperiodate (25 ml) was directly added and stirring continued for 1 h. The reaction was quenched by the addition of water (300 ml), the aqueous phase extracted with diethyl ether (3×300 ml) and the combined organic extracts washed successively with satd. sodium bicarbonate solution (200 ml), water (300 ml), dried over sodium sulphate and evaporated to give a dark solid. Attempts to purify this through literature procedures were not fully successful and after numerous combinations, purification using silica gel chromatography using a mixture of 5% acetone/DCM worked the best and the desired product was obtained as a brown powder 0.3 g (60%). This compound was characterised according to the literature.

### Methyl 3-devinyl-3-carboxypyropheophorbide a (33)

A mixture of pyropheophorbide-d (32) (165 mg, 0.3 mmol), sulfamic acid (175 mg, 1.78 mmol) 2-methyl-2-butene (2M soln. In THF, 7.5 ml) and water (0.75 ml) was stirred at room temperature for 10 min. under argon. To this mixture, sodium chlorite (135 mg, 0.015 mmol) dissolved in water (0.6 ml) was added drop wise over 10 min. Once addition was complete, the reaction was stirred for a further 30 min. at room temperature when TLC [silica gel: 5% MeOH/DCM] indicated that the reaction was complete. The reaction mixture was poured into water (100 ml), extracted with chloroform (2×100 ml), the combined organic layers were then washed with water (3×), dried (Na₂SO₄) and evaporated. The residue was purified by column chromatography [silica gel: 5% MeOH/DCM] to give pure acid (33) as a brown solid, 51 mg, 30%, R_{f} 0.25 (R_{f} 0.61 for compound 32). UV/V is(CHCl₃) λₘₐₓ 419, 384, 685, 649, 517, 625. MS ES (m/z) (Calc. For C33H34N4O5, 566.61) Found 567.2607 (M⁺+H).

### Methyl 3-devinyl-3-carboxybenzyl-3,4,5-(triethylene glycol monomethylether) pyropheophorbide a (34)

To a stirred solution of (33) (35 mg, 0.066 mmol) in a mixture of dry DCM/THF (9:1, 10 ml) under argon, 3,4,5-(triethyleneglycol monomethyl ether) benzylalcohol (4) (56.5 mg, 0.095 mmol), diisopropylcarbodiimide (64 μl, 0.412 mmol), DMAP (30 mg, 0.25 mmol), DPTS (71 mg, 0.24 mmol) were added and stirred for approx. 10 min. after which anhydrous N-ethyldiisopropylamine (39 μl, 0.223 mol) was added and stirring continued for a further 12 h. The reaction mixture was evaporated to dryness and the residue purified by column chromatography [silica gel: 10% MeOH/CHCl₃) to give the desired pure (34) as a red/brown oil 46 mg, 61%, R_{f} 0.43 (R_{f} 0.34 for compound 33, 10% MeOH/CHCl₃). UV/Vis(DCM) λₘₐₓ 418, 384, 683, 648, 516, 621. MS ES (m/z) (Calc. For C31H82N4O17, 1142.32) Found 1143.5753 (M⁺+H). Also Found 1165.5573 (M⁺+Na).

### Methyl 3-devinyl-3-carboxybenzyl-3,4,5-(triethylene glycol monomethylether) meso 5-bromopyropheophorbide a (35)

To a stirred solution of (34) (40 mg, 0.035 mmol) in dry DCM (15 ml) under argon, anhydrous pyridine (28.3 μl, 0.35 mmol) was added followed by pyridimium perbromide (14.5 mg, 0.045 mmol) which had been dried on a high vacuum pump prior to use. The reaction mixture was stirred at room temperature during which it was followed by UV/Vis. spectroscopy and was complete within 20 min. The reaction mixture was evaporated to dryness and purified by column chromatography [silica gel: 5% MeOH/CHCl₃] to give pure (35) as a red/purple oil 13 mg, 30%, R_{f} 0.14. UV/Vis(DCM) λₘₐₓ 417, 388, 686, 521, 565, 627. MS ES (m/z) 1222 (M⁺) 1245 (M⁺+Na,100%).

### 3-devinyl-3-carboxypyropheophorbide a (37)

Methyl 3-devinyl-3-carboxypyropheophorbide a (33) (15 mg, 0.0272 mmol) was dissolved in a very small amount of dry THF and placed under argon. To this solution conc. hydrochloric acid (10 ml) was slowly added and the resulting green solution stirred at room temperature, shielded from light for 12 h. The reaction was quenched by slowly dropping the reaction mixture onto a large amount of crushed ice and a colour was observed. The ice/water was extracted with chloroform (3×50 ml), dried and evaporated to give a brown solid 11.2 mg, 74%, R_{f} 0.23 (10% MeOH/CHCl₃). UV/Vis (CHCl₃) λₘₐₓ 419, 383, 685, 549, 517, 626. MS ES (m/z) (Calc. For C32H32N4O5, 553.2451) Found 553.2449 (M⁺).

### Meso pyropheophorbide a-3,4,5-(triethylene glycol monmethylether) 3-devinyl-3-carboxybenzyl-3,4,5-(triethylene glycol monomethylether) pyropheophorbide a (38)

3-devinyl-3-carboxypyropheophorbide a (37) (10 mg, 0.019 mmol) in a mixture of dry DCM/THF (9:1, 5 ml) under argon, 3,4,5-(triethyleneglycol monomethyl ether) benzylalcohol (4) (33.1 mg, 0.056 mmol), diisopropylcarbodiimide (37.4 μl, 0.241 mmol), DMAP (17.7 mg, 0.145 mmol), DPTS (41.6 mg, 0.141 mmol) were added and stirred for approx. 10 min. after which anhydrous N-ethyldiisopropylamine (22.6 μl, 0.13 mol) was added and stirring continued for a further 12 h. The reaction mixture was evaporated to dryness and the residue purified by column chromatography [silica gel: 10% MeOH/CHCl₃) to give the desired pure (34) as a red/brown crystalline solid 17.3 mg, 55%, R_{f} 0.43. UV/Vis (DCM) λₘₐₓ 416, 384, 683, 648, 516, 621. MS ES (m/z) 1706.9 (M⁺, 1729.9 (M⁺+Na).

### Meso 5-bromopyropheophorbide a-3,4,5-(triethylene glycol monmethylether) 3-devinyl-3-carboxybenzyl-3,4,5-(triethylene glycol monomethylether) pyropheophorbide a (39)

Compound (38) (5 mg, 0.00293 mmol) was dissolved in dry DCM (2 ml) and placed under argon To this stirred solution, anhydrous pyridine (2.4 μl, 0.029 mmol) was added followed by pyridinium perbromide (1.2 mg, 0.0038 mmol). The reaction mixture was stirred at room temperature during which it was followed by UV/Vis. spectroscopy and was complete within 20 min. The reaction mixture was evaporated to dryness and purified by column chromotography [silica gel: 10% MeOH/CHCl₃] to give pure (39) as a red/purple solid 2 mg, 38%, R_{f} 0.14. UV/Vis(DCM) λₘₐₓ 417, 389, 687, 521, 564, 627. MS ES (m/z) (Calc. For C88H127 BrN4O29, 1185.8253) Found 1185.8661 (M⁺+H). Also Found 1231.5673 (M⁺+2Na).

### Meso Pyropheophorbide a propargylamide (45)

Meso pyropheophorbide a (100 mg, 0.185 mmol) was dissolved dry DCM/20% THF (10 ml) and placed under a blanket of argon. To this stirred solution N-hydroxy succinimide (27 mg, 0.233 mmol) followed by diiisopropylcarbodiimide, DIC (37.4 μl, 0.242 mmol) were added and the resulting mixture stirred at room temperature, shielded from light for 12 h, when TLC [silica gel: 5% MeOH/CHCl₃, R_{f} 0.47] showed the presence of the active ester and consumption of all the starting material. At this point a 2-fold excess of propargyl amine (25.6 μl, 0.373 mmol) was added and stirring continued for a further 6 h. TLC [silica gel: 5% MeOH/CHCl₃) showed that all of the active ester had been consumed and that a new major component with an R_{f} 0.22 was present. The reaction mixture was evaporated to give a dark-green solid. This was triturated overnight with hexane, filtered and dried to give a dark-green powder in quantitative yield. UV/Vis DCM) λₘₐₓ 410, 395, 656, 601, 503, 635. MS ES (m/z) (Calc. For C36H40N5O2, 574.3182) Found 574.3186 (M⁺+H).

### Meso 5-Bromo pyropheophorbide a propargylamide (46)

Meso pyropheophorbide propargylamide (45) (50 mg, 0.087 mmol) was dissolved in dry DCM (20 ml) and placed under argon To this stirred solution, anhydrous pyridine (70.5 μl, 0.871 mmol) was added followed by pyridinium perbromide (36.2 mg, 0.113 mmol). The reaction mixture was stirred at room temperature during which it was followed by UV/Vis. spectroscopy and was complete within 20 min. The reaction mixture was evaporated to dryness and purified by column chromatography [silica gel: 5% MeOH/CHCl₃] to give pure (36) as a purple powder 21 mg, 37%, R_{f} 0.5 UV/Vis (DCM) λₘₐₓ 415, 668, 549, 611, 516. MS ES (m/z) (Calc. For C36H39BrN5O2, 652.2287) Found 652.2290 (M⁺+H).

### Meso Pyropheophorbide a N-(6-N′-t-butoxycarbonyl-aminohexyl amide (59)

Meso pyropheophorbide a (100 mg, 0.185 mmol) was dissolved dry DCM/20% THF (10 ml) and placed under a blanket of argon. To this stirred solution N-hydroxy succinimide (27 mg, 0.233 mmol) followed by diiisopropylcarbodiimide, DIC (37.4 μl, 0.242 mmol) were added and the resulting mixture stirred at room temperature, shielded from light for 12 h, when TLC [silica gel: 5% MeOH/CHCl₃, R_{f} 0.47] showed the presence of the active ester and consumption of all the starting material. At this point a 2-fold excess of N-Boc-1,6-diamine (84 μl, 0.373 mmol) was added and stirring continued for a further 6 h. TLC [silica gel: 5% MeOH/CHCl₃) showed that all of the active ester had been consumed and that a new major component with an R_{f} 0.21 was present. The reaction mixture was evaporated to give a dark-green solid. This was triturated overnight with hexane, filtered and dried to give a dark-green powder 95 mg, 68%. UV/Vis (DCM) λₘₐₓ 410, 395, 656, 601, 503, 635. MS ES (m/z) (Calc. For C36H39N5O2, 735.4598) Found 735.4603 (M⁺+H).

### Meso 5-Bromo pyropheophorbide a N-(6-N′-t-butoxycarbonyl-aminohexyl amide (60)

### Meso Pyropheophorbide a N-(6-N′-t-butoxycarbonyl-aminohexyl amide (59)

(65 mg, 0.088 mmol) was dissolved in dry DCM (20 ml) and placed under argon To this stirred solution, anhydrous pyridine (71.5 μl, 0.884 mmol) was added followed by pyridinium perbromide (36.8 mg, 0.115 mmol). The reaction mixture was stirred at room temperature during which it was followed by UV/Vis. spectroscopy and was complete within 20 min. The reaction mixture was evaporated to dryness and purified by column chromatography [silica gel: 5% MeOH/CHCl₃] to give pure (60) as a purple powder 22 mg, 31%, R_{f} 0.46. UV/Vis (DCM) λₘₐₓ 416, 669, 549, 611, 516. MS ES (m/z) (Calc. For C44H58BrN6O2, 813.3703) Found 813.3706 (M⁺+H).

### Meso 5-Bromo pyropheophorbide a N-(6-aminohexyl) imide Gd(III)-DTPA (62)

Meso 5-Bromo pyropheophorbide a N-(6-N′-t-butoxycarbonyl-aminohexyl amide (60) (10 mg, 0.012 mmol) was dissolved in dry DCM and TFA (1 ml) was added. This results in an immediate colour change from purple to green (UV/Vis (DCM) 423, 662, 611, 581). The reaction mixture was stirred for 45 min. at room temperature and then neutralised by the addition of satd. sodium bicarbonate solution, the colour changing back to the original purple. TLC [silica gel: 10% MeOH/CHCl₃) indicated consumption of all the starting protected amine (60) and the presence of the free amine as spot on the base line. DCM (100 ml) was added to the reaction mixture and back extracted. The combined organic extracts were then dried (Na₂SO₄) and evaporated. Diethylenetriamine-pentaacetic acid dianhydride caDTPA(2.5 mg, 0.006 mmol) was dissolved in dry DMF (0.5 ml) and triethylamine (100 μl) added.To this mixture a solution of the amine dissolved in dry DMF (0.5 ml) was then added dropwise. The resulting mixture was stirred at room temperature for 12 h, water (1 ml) was then added and the reaction mixture stirred for a further 45 min. After which a large volume of diethylether was added and the mixture left overnight in the fridge. The precipitated solid was collected, washed with MeOH, diethylether and DCM, dried to give a purple powder 8 mg. To this solid, pyridine (1.5 ml) and methanol (1 ml) were added followed by a solution of GdCl₃ hexahydrate (2.8 mg, 0.0215 mmol) in water and stirred for 12 h at room temperature. After removing the solvent under vacuum at 45° C., water was added (2 ml) and the mixture filtered, washed with water, acetonitrile and dried to give a purple solid. UV/Vis (DCM) λₘₐₓ 417, 669, 549, 611, 516. MS FAB (m/z) 1227.3315

### Vic-7,8-Dihydroxymethyl meso 5-bromopyropheophorbide a-3,4,5-(triethylene glycol monomethylether) benzyl ester (48)

Pyridine (10 μl) was added to a solution of (9) (10 mg, 0.0083 mmol) in dry DCM (2 ml). To this osmium tetroxide (10 mg) was added and the resulting mixture stirred at room temperature whilst being monitored by UV/Vis spectroscopy. The reaction was complete within 6 h as indicated by UV/Vis spectroscopy and TLC [silica gel: 5% MeOH/CHCl₃, R_{f} 0.38 (ft 0.5 for compound 9). The reaction was quenched by bubbling H₂₅ gas into the reaction mixture for 5 min. to decompose any unreacted OsO₄. The reaction was then allowed to evaporate to dryness overnight, then re-dissolved in DCM and filtered through a short pad of Celite. The residue after evaporation was purified by chromatography to give the bacteriochlorin (48) as a purple sticky solid 7.7 mg, 75%. UV/Vis (DCM) λₘₐₓ 367, 714, 532, 667, 417, 500. MS ES (m/z) 1247. 51 (M⁺+23, 100%)

### Methyl 3-ethynylpyropheophorbide a (41)

To a solution of pyropheophorbide d (100 mg, 0.18 mmol) in dry THF (15 ml) and dry methanol (15 ml) were added CsCO₃ (100 mg, 0.31 mmol) and the Bestmann-Ohira reagent (168 mg, 0.88 mmol). The reaction was stirred at room temperature under argon and monitored by UV/Vis. Spectroscopy until the Q-band peak at 693 nm completely disappeared (approx. 5 h). The reaction was quenched by pouring into aqueous sodium bicarbonate solution, and extracted with DCM. The DCM extract was washed with water, dried (Na₂SO₄) and concentrated. The crude was purified by column chromatography [silica gel: 7% diethyl ether/DCM] to give the desired compound as a shiny crystalline solid 41 mg, 42%, R_{f} 0.3. UV/Vis (DCM) λₘₐₓ 412, 675, 616, 510, 638. MS ES (m/z) (Calc. For C34H35N4O3, 547.2709) Found 547.2713 (M⁺+H).

1-Amino-4-pentyne (52) can be made using standard chemistry procedures, starting with 1-hydroxy-4-pentyne (49) (commercially available), converting this hydroxyl compound to its mesylate (methane sulphonyl chloride, Et₃N in dry ether), converting the mesylate to the azide (sodium azide, DMF), and reduction of the azide to the amine (triphenyl phosphine, THF).

### N-pentynemaleimide (54)

This was synthesised from 1-Amino-4-pentyne using an analogous procedure for the preparation of N-propargylmaleimide (PCT 2006/050192)

### EXAMPLE 4

### MRI Active Derivatives

Targeted drugs hold great promise for the future treatment of cancer. However, there are many challenges for effective evaluation of such molecules in pre-clinical (animal) and clinical studies. Issues such as administering the appropriate biological dose, the correct dose schedule, selecting and diagnosing the patient population who are most likely going to respond to the treatment, understanding and assessing the tumour response (especially early indications) all need to be addressed in this new era of molecularly-targeted drug therapy and ‘personalised medicine’.

Anatomical imaging will remain key to cancer medicine but molecular imaging will provide fantastic opportunities to make drug development more successful and treatment more effective. Targeted, non-invasive and quantitative imaging approaches will greatly compliment new drug development and will be able to complement current imaging approaches. Magnetic resonance imaging (MRI) is a safe yet powerful diagnostic technique for visualizing soft tissue often with the aid of paramagnetic contrast agents. Chelate structures that contain Gd3⁺ or other paramagnetic ions such as manganese (2⁺ or 3⁺) and iron 3⁺ improve imaging contrast by increasing the longitudinal relaxation time (T1) of proximal water protons, which appear brighter in the T1-weighted image.

Magnevist™ and Omniscan™ are two frequently used commercial products for MRI that contain Gd3⁺ chelated by DTPA. However, these and other FDA-approved small molecule chelates not only have low retention times in vivo, but they also suffer from an inherent lack of sensitivity for application in cellular imaging and medical diagnostics.

The first step toward improving the diagnostic capability of contrast agents is to make them target specific and to accumulate in specific biological locations, and antibodies represent a natural way to achieve both these aims. Initial efforts to create targeted contrast agents for MRI involved direct conjugation of a contrast agent (typically Gd-DTPA) onto a whole antibody.

However, this method did not prove to be very successful owing to the relative low sensitivity of MRI and the low concentration of most cellular targets. Although Mn(III) porphyrins have been studied as potential contrast agents (CA), very little has been published on the insertion of Mn into chlorophyll-a derivatives like PPa.

Our initial studies have looked at the insertion of Mn into PPa, (scheme 16). Metallation was achieved in high yields using a combination of Mn(II)Acetate in glacial acetic acid, the resulting Mn(II) derivative oxidising to give the Mn(III) complex (55) as an isolable product which was then converted into its N-hydroxysuccinimide derivative (56). The insertion manganese has dramatic effects on the UV/Vis spectrum of PPa, with the Soret band splitting into two broad peaks (375 and 476 nm) and the Q-bands becoming very broad.

The same synthetic methodology was applied in inserting manganese into compound (10) to give the water soluble derivative (57) whose UV/Visible spectrum showed a splitting of the Soret band (373 and 472 nm) and both a red shifting (690 nm) and broadening of the furthest Q-band. The active ester derivative (58) was synthesised and isolated as a dark-green solid after chromatography on silica using 20% MeOH/CHCl₃.

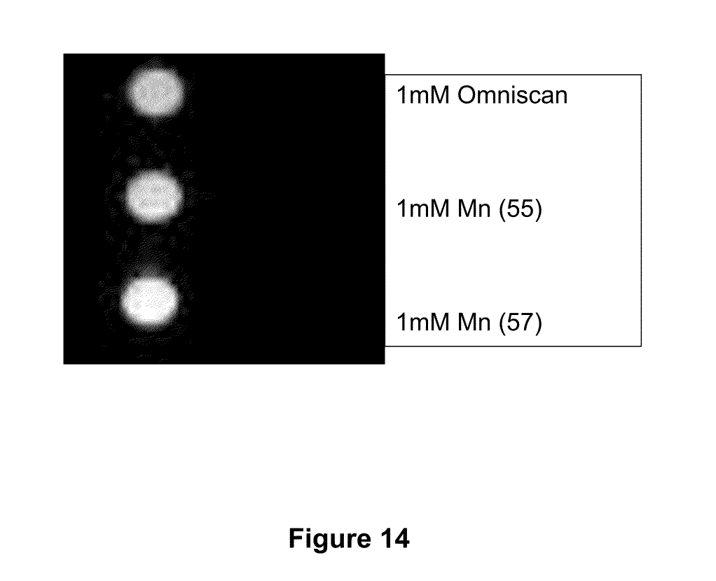

One mM solutions in saline buffer was imaged in a 4.7T small MRI imaging chamber using a T1 protocol, and compared to a known MRI imaging agent, Omniscan. All three gave MRI-signals with the two PPa derivatives showing more intense signals than Omniscan. (FIG. 14)

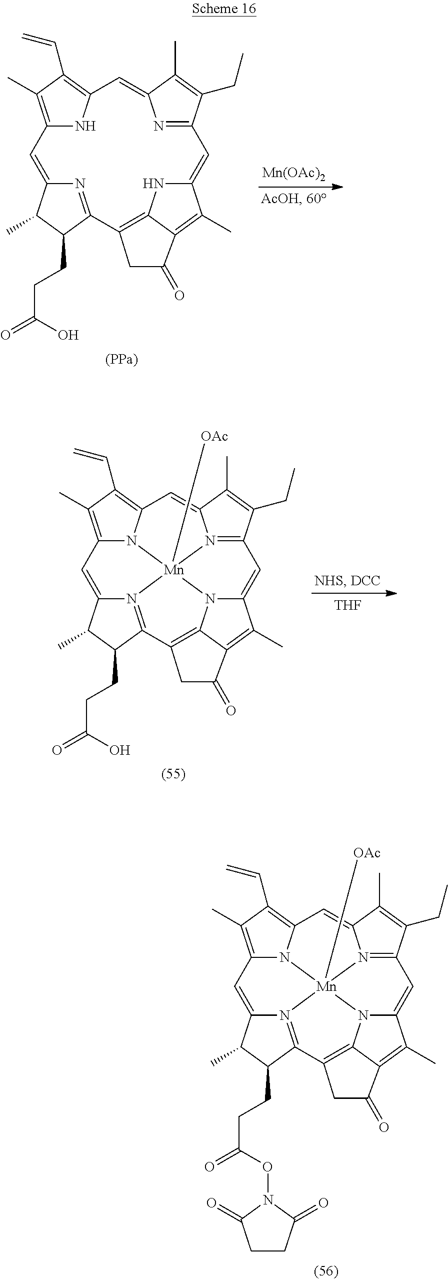

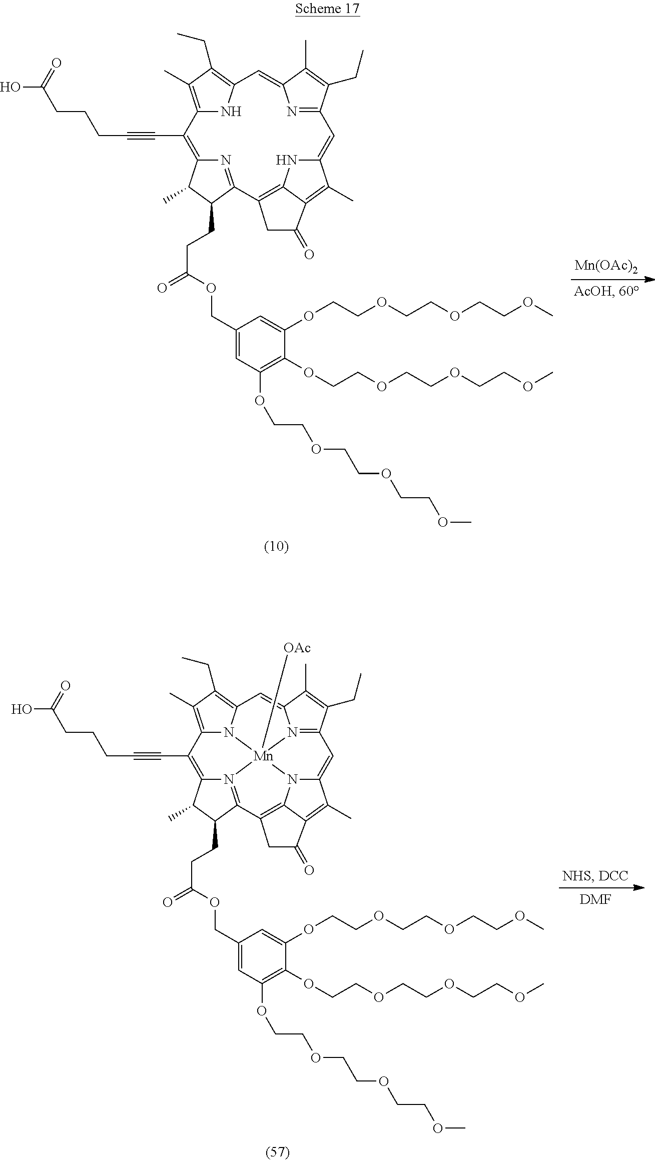

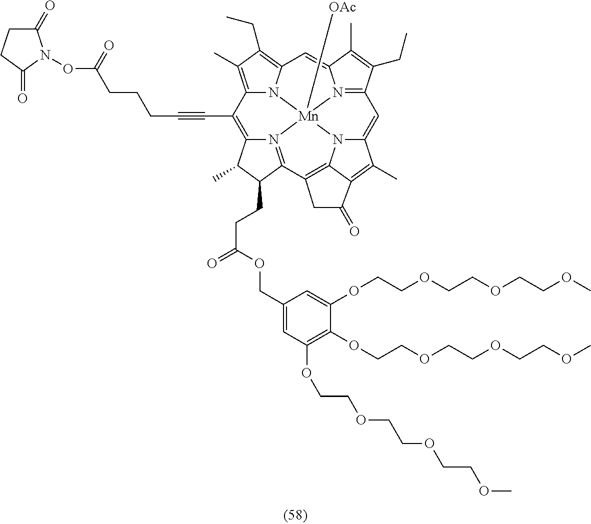

The advantages of antibody targeting can therefore be combined with new bifunctional agents that combine two modalities into a single cost-effective ‘see and treat’ approach, namely, a single agent that can be used for CA enhanced MR imaging as well as targeted PDT.

The insertion of a paramagnetic metal like Mn into the porphyrin core can in some instances result in the quenching of the PPa fluorescence. To overcome this limitation we complexed the paramagnetic metal using a suitable ligand attached to the porphyrin periphery, (scheme 18).

This approach enables the expansion into both gadolinium [Gd(III)] complexes of PPa and the ability to conjugate these complexes to antibodies. The Gadolinium (III) ion with its seven unpaired electrons and large paramagnetic moment is the most widely used contrast agent. Meso-PPa (17) was esterified with a mono Boc-protected hexyldiamine using the two-step procedure developed by us, giving the amide (59) as a dark-green powder in high yields.

This involves pre-forming the N-hydroxysuccinimide derivative of the acid in situ by reacting the acid with NHS in the presence of a dehydrating agent such as DCC or DIC. The reaction was followed by TLC and once all the starting acid (17) was consumed and a spot with a higher R_{f} had appeared, the amine was added in one go and stirring at room temperature continued for a further 5 hr, when the reaction was complete as judged by TLC. This was then brominated to give the meso 5-bromo derivative (60) in good yields.

Immediately prior to the next step, the Boc group was removed using TFA to give the free amine, the reaction mixture was evaporated and the resulting crude material stirred with DTPA bis-anhydride in dry DMF to give diethylenetriaminepentacetic acid derivative (61) in good yields. The DTPA moiety has long been used for the formation of various Gd(III) chelates and by stirring compound (61) with aqueous GdCl₃ in pyridine and methanol gave the gadolinium complex (62), scheme 18.

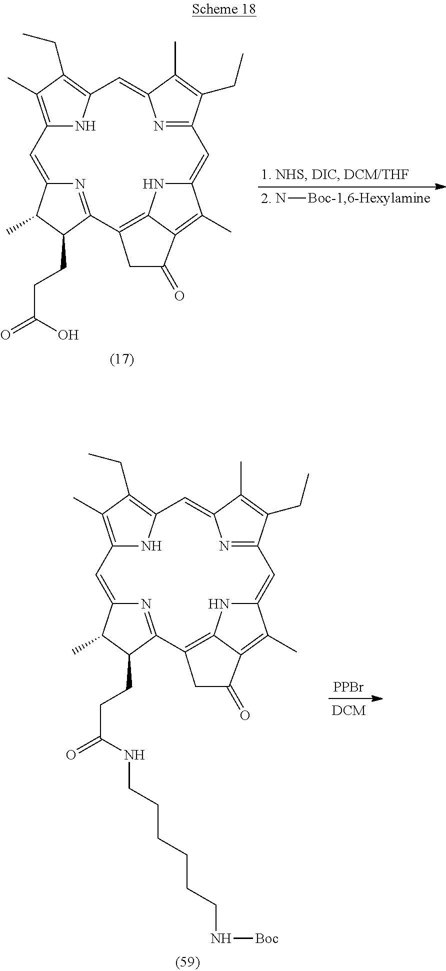

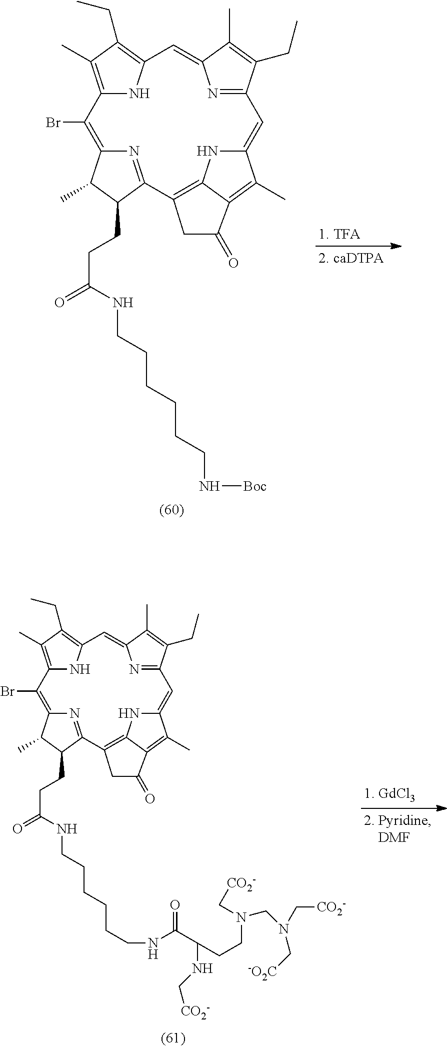

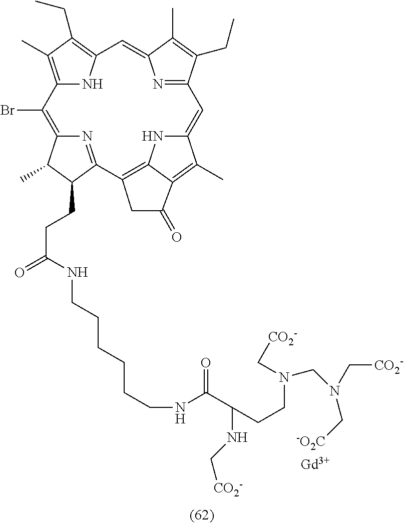

### Synthetic Routes and Methods

The manipulation of air and/or water sensitive compounds was carried out using standard Schlenk techniques. DCM and triethylamine were dried by distilling from CaH₂ and dry THF was obtained by distillation from sodium/benzophenone. All other reagents were used as supplied by commercial agents unless stated otherwise.

Analytical thin layer chromatography (TLC) was carried out on Merck glass backed silica gel 60 GF₂₅₄ plates or aluminium backed aluminium oxide (neutral) and visualisation when required was achieved using UV light or in some cases a chemical staining agent was used. Column chromatography was carried out on silica gel 60 or aluminium oxide(neutral or basic) deactivated with 5% water (referred to as Brockmann grade III) using a positive pressure of air. Where mixtures of solvents were used ratios reported are by volume.

NMR spectra were recorded at ambient probe temperature using a Bruker DPX400 (400 MHz). Chemical shifts are quoted as parts per million (ppm) with CDCl₃ as internal standard (for ¹H NMR, 7.26 ppm) and coupling constants (J) are quoted in Hertz (Hz). UV/Vis spectra were recorded on a Hewlett Packard 8450 diode array spectrometer. Mass spectra were carried out using a number of techniques and only molecular ions and major peaks are reported.

### Manganese(III)-Pyropheophorbide-a (55)

To a stirred solution of PPa (50 mg, 0.0935 mmol) in glacial acetic acid (2 ml), Mn(OAc)₂.4H₂O (110 mg, 0.468 mmol) dissolved in glacial acetic acid (3 ml) was added and the reaction mixture heated at 60° C. for 2 h when UV/Vis. spectroscopy showed completion of the metallation-the colour of the solution goes a vivid clear green colour. After cooling, the reaction mixture was transferred to a conical flask (100 ml) using fresh acetic acid, to this a large volume of hexane was added, the contents shaken vigorously, allowed to settle after which the hexane layer was decanted off. This procedure was repeated 4×, after which the green oily layer became granular. This process was repeated 3× using diethyl ether as solvent. Throughout both processes the pungent smell of the acetic acid gradually disappears. To the obtained green solid DCM (100 ml) was added and the suspension transferred to a separating funnel, water was then added and shaken vigorously, this process was repeated until a majority of the solid had dissolved in the organic layer. The combined organic layer was evaporated to a give a dark-green amorphous powder. 46 mg (76%).Due to paramagnetic broadening we were unable to carryout NMR spectroscopy. UV/Vis (THF) λₘₐₓ 369, 472, 684, 439, 654, 621, 539. MS ES (m/z) 587 (M⁺-OAc, 100%)

### Manganese(III) Pyropheophorbide-a succinimido ester (56)

To a dark-green solution of manganese (III) PPa (55) (6 mg, 0.0093 mmol) in anhydrous THF (5 ml), N-hydroxysuccinimide (1.6 mg, 0.14 mmol) followed by DCC (3.83 mg, 0.187 mmol) was added. The resulting mixture was shielded from light and stirred at room temperature under argon for 12 h. TLC [silica gel: 20% MeOH/CHCl₃] showed slight differences in the R_{f} between (55, R_{f} 0.31) and (56, R_{f} 0.25). The reaction was evaporated to and the dark-green/brown residue was dissolved in a minimum of chloroform and dropped into a large stirred volume of hexane causing an immediate flocculent precipitate. This was allowed to settle and, the majority of the hexane decanted off and replaced by fresh hexane and stirred for 15 min. allowed to settle and decanted off, repeated a total of 4×. The remaining green residue was dried to a give a green solid. 5 mg (72%). UV/Vis (THF) λₘₐₓ 368, 472, 685, 438, 655, 621, 539. MS ES (m/z) 684 (M⁺-OAc, 100%).

### Manganese(III)-5-ethynyl hexynoic acid pyropheophorbide a-3,4,5-(triethylnene glycol monomethyl ether) benzyl ester (57)

To a solution of compound (10) (9.5 mg, 0.0078 mmol) in glacial acetic acid (2 ml) Mn(OAc)₂.4H₂O (9.5 mg, 0.0039 mmol) was added and the resulting mixture heated at 60° C. for approximately 2 h. The colour of the reaction changes from dark-purple to a vivid green, this is reflected in dramatic changes to the UV/Vis spectrum. The reaction was evaporated and the residue passed though a short silica column eluting with 20% MeOH/CHCl₃. A green sticky residue was obtained which washed with hexane repeatedly and dried to give a green oil. 8 mg (79%). UV/Vis (MeOH) λₘₐₓ 373, 690, 685, 472, 645, 587. MS ES (m/z) 1271 (M⁺-OAc, 100%).

### Manganese(III)-5-ethynyl hexynoyl succinimido ester pyropheophorbide a-3,4,5-(triethylnene glycol monomethyl ether) benzyl ester (58)

Compound (57) (5.8 mg, 0.0043 mmol) was dissolved in dry DMF (0.5 ml) and N-hydroxysuccinimide (1 mg, 0.0052 mmol) and DCC (1.4 mg, 0.0065 mmol) were added and the reaction mixture stirred overnight at room temperature under argon, during which some precipitation was observed. The reaction mixture was evaporated under high vacuum to give a ‘murky’ green residue which was extracted with THF and these extracts combined and evaporated to give a green semi-solid 2.9 mg, 47%. UV/Vis (MeOH) λₘₐₓ 371, 693, 685, 471, 646, 587.

### EXAMPLE 5

### Biological Testing of Phototoxicity of Compounds and Photoimmunoconjugates

### Methods

### Cell Culture

Two different human-derived tumour cell lines: SKOV3 and KB were obtained from the European Collection of Cell Cultures (ECACC) and cultured in Dulbecco's modified Eagle's medium (DMEM) with 10% foetal bovine serum, penicillin and streptomycin antibiotics (1%) and passaged when 70-90% confluent in 75 cm² flasks. The cells were maintained at 37° C. in a humidified 5% CO₂ atmosphere.

Expression and Purification of C6.5 scFv

C6.5 scFv was obtained from Prof J. Marks (University of California, San Francisco) in pUC119 and expressed in XL1 blue cells (Adams et al, 2000). The C6.5 scFv was engineered to remove a lysine-100 in the antibody binding site. This was to reduce the possibility of forming PICs of reduced immuno-reactivity (Adams et al, 2000). Cultures were grown as above.

Purification of C6.5 was carried out as follows. Cultures of 500 ml of 2TY media containing 100 pg/ml ampicillin were grown at 30° C. and induced at an optical density (600 nm) of 0.7 by adding IPTG to a final concentration of 1 mM. C6.5 scFv was recovered from the filtered culture supernatant ultrafiltration and concentration and dialysed against PBS buffer exhaustively. The crude protein was applied to a chelating Sepharose column charged with NiCl₂. The column was washed in binding buffer supplemented with imidazole 10 mM-60 mM and the pure scFv was eluted in binding buffer with 100 mM imidazole. Purified protein was either concentrated to 1 mg/ml protein using 25 ml spin concentrators and stored in 10% glycerol at −80° C., or used for couplings straight after purification without concentrating.

### Synthesis of scFv-Photosensitiser Photo-Immunoconjugates (PICs)

The photosensitiser succinimidyl ester was re-suspended in 100% DMSO and added at a concentration of 52.8 μM to 3.3 μM scFv in PBS containing 6% acetonitrile and with continuous stirring at 4° C. for 120 min. The photoimmunoconjugates (PICs) were then dialysed against PBS with two buffer changes, followed by centrifugation. SDS-PAGE analyses was carried and stained with coomassie blue. Non-stained gels were transferred using a semi-dry blotting apparatus (Biorad) onto nitrocellulose and gently dried.

Fluorescence was visualised by exciting the photosensitisers on the blot on a short wavelength UV-transilluminator. The fluorescence of free and conjugated photosensitiser was used to determine the level of non-covalently bound photosensitisers (typically 30-50%). A standard curve of photosensitiser absorbance at 670 nm was used to determine the photosensitiser content in the photo-immunoconjugate. This was used to determine the number of photosensitiser molecules covalently coupled to the antibody (typically 6-10:1 photosensitiser:scFv)

### In Vitro Cytotoxicity

Cells were trypsinised and seeded at 2×10³ cells/well for KB and 3×10³ cells/well for SKOV3 into 96-well plates and incubated over two nights for KB and overnight for SKOV3 at 37° C. and 5% CO₂. The cells were then washed once in phenol red free DMEM and appropriately diluted photosensitiser solution or photo-immunoconjugate (in DMEM, 2% DMSO) was added to the appropriate wells under subdued lighting. DMEM or Triton X-100 (1%) were added to control wells. After 2 hrs incubation in the dark at 37° C., 5% CO₂, cells were washed three times with PBS and 1000 of DMEM was added to each well.

Wells were exposed to light from a High Powered-Devices 670 nm laser used at 0.5 W for 10s. Controls included wells with photosensitiser added and no exposure to light, or DMEM added and exposure to light as well as Triton X-100 (1%) with or without light exposure. Cells that had no photosensitiser added and no exposure to light were included as overall controls. Cells were incubated in the dark at 37° C., 5% CO₂ for 48 h after which time, a cell titre assay was performed according to the manufacturer's instructions. The Promega Cell Titre-96 system was used which involves the conversion by live cells of a tetrazolium compound (MTS) into a formazan dye which is measurable by its absorbance at 490 nm.

### Imaging by Confocal Microscopy

Imaging took place at the FILM Imperial College London on a LEICA SP5MP inverted confocal microscope, at 37° C. and under a CO₂ supplemented atmosphere. The water objective (63×) was used in all the experiments. Images were processed using the Leica software, Image J and Powerpoint.

Note: in any experiment where a photosensitiser is used on cells, the experiment is done under subdued lighting exposing the cells to as little light as possible and incubating wrapped in foil. Following addition of PS to cells, cells were no longer observed on the microscope.

Cells were washed (3×100u1 media) and plated 25000 cells per chamber in 200 μl of phenol red free DMEM (10% FBS, 1 P/S) and allowed to grow overnight (it was found that less confluent (60-70%) cells attached better for imaging experiments). Cells were plated on a Labt-Tek® 8 Chambered #1.0 Borosilicate Coverglass System. With some cell lines, it is often useful to use an attachment factor (200 μl, 30 mins, 37 0 C) to coat the wells first.

### Cellular Staining Using Photosensitisers (PPa, PS1 and PS4)

Photosensitiser solutions in phenol red free DMEM (10% FBS, 1% P/S, 0.5% DMSO) were prepared fresh and pre-warmed to 37° C. for 15 mins. The cells were washed with media (1×200 μl) before adding the solutions (200 μl per chamber). Unless otherwise stated, the cells were grown over 20 hrs in a humidified atmosphere (37° C., 5% CO₂). The cells were washed (2×200 μl) with prewarmed phenol red free DMEM

### Lysosomal Staining

Lysotracker® Green DND-26 was diluted according to manufacturers indications (1 mM stock) and further diluted to twice the working concentration in DMEM (10% FBS, 1% P/S). For use for single colour staining, this was diluted with DMEM (10% FBS, 1% P/S) to a final concentration of 1 μM. When it was used for two colour staining with photosensitisers, a 2 μM solution was twice diluted in photosensitiser solution to give a 1 μM final concentration.

The cells were incubated for 15 mins at 4° C. and a further 30 mins at 37° C. before replacing with fresh medium and imaging.

### Mitochondrial Staining

MitoTracker® Green was diluted according to manufacturers indications (1 mM stock) and further diluted to twice the working concentration in DMEM (10% FBS, 1% P/S). For use for single colour staining, this was diluted with DMEM (10% FBS, 1% P/S) to a final concentration of 1 μM. When it was used for two colour staining with photosensitisers, a 2 μM solution was twice diluted in photosensitiser solution to give a 1 μM final concentration.

The cells were incubated for 15 mins at 4° C. and a further 1 hr at 37° C. before replacing with fresh medium and imaging.

### Endoplasmic Reticulum Staining

ER-Tracker™ Green (BODIPY® FL glibenclamide) was diluted according to manufacturer's indications (1 mM stock) and further diluted to twice the working concentration in HBSS buffer (+2% HEPES). For use for single colour staining, this was diluted with HBSS (+2% HEPES) to a final concentration of 3 μM. When it was used for two colour staining with photosensitisers, a 6 μM solution was twice diluted in photosensitiser solution to give a 3 μM final concentration.

The cells were incubated for 15 mins at 4° C. and a further 1 hr at 37° C. before replacing with fresh buffer and imaging.

### Golgi Apparatus Staining

Bodipy® FL C₅-ceramide complexed to BSA was diluted according to manufacturer's indications (0.5 mM stock) and further diluted to twice the working concentration in HBSS buffer (+2% HEPES). For use for single colour staining, this was diluted with HBSS (+2% HEPES) to a final concentration of 5 μM. When it was used for two colour staining with photosensitisers, a 10 μM solution was twice diluted in photosensitiser solution to give a 5 μM final concentration. The cells were incubated for 15 mins at 4° C. and a further 30 mins at 37° C. before washing with cold HBSS/HEPES (3×100 μl) and replacing with either HBSS/HEPES or photosensitiser solution and incubating at 37° C. for a further 30 mins before replacing with fresh buffer and imaging.

### MR-Properties of Novel PPa-Derivatives

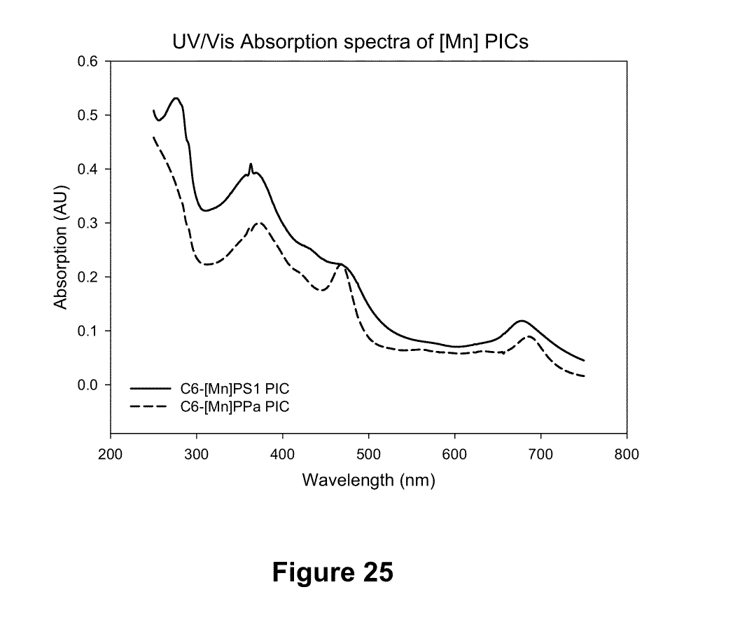

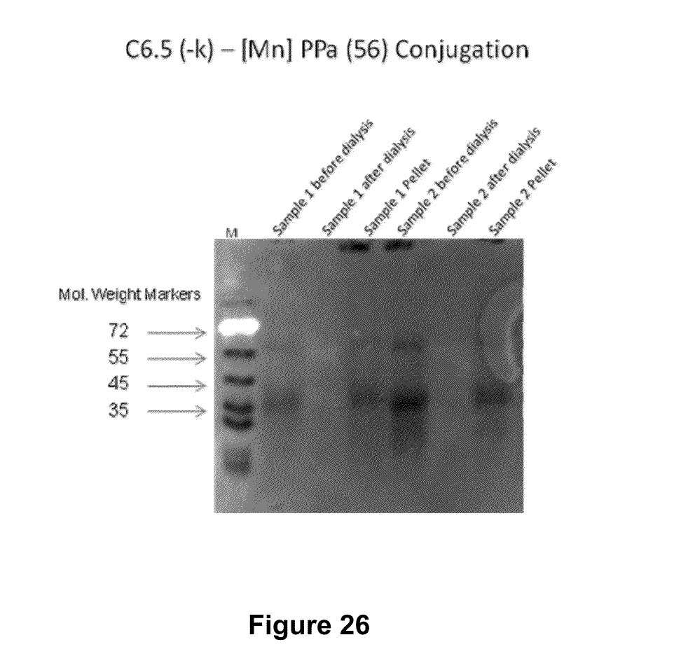

The basic MRI experiments consisted of T1 relaxation measurements of the samples. We used inversion recovery pulse sequence with adiabatic inversion pulse. T1 relaxivity was compared to standards (such Omniscan in water) and sample concentration. The scFv conjugates of compounds (56) and (58) were prepared as above, the UV/Vis spectrum of both immunoconjugates (FIG. 25), we can see both the protein absorption at 280 nm and the characteristic absorption profile of the porphyrin. The Mn(56) conjugate was less soluble in buffer than the corresponding Mn(58) conjugate reflecting the fact that the addition of the TriPEG groups onto PPa results in more aqueous soluble photosensitisers. A gel of C6-Mn(56) immunoconjugate (FIG. 26) clearly shows the presence of the conjugate as band around 35 kD before dialysis.

TABLE 3
Photophysical parameters of the photosensitisers under study

| compound | PPa | 10 | 31C |
|---|---|---|---|
| λₘₐₓ(em)/nm | 675, 722 | 677, 731 | 690, 755 (shoulder) |
| φ_{f} | 0.3 | 0.26 | 0.15 |
| φ_{Δ} | 0.5 | 0.56 | 0.73 |
| τ_{Δ}/μs | 30 | 30 | 30 |

λₘₐₓ(em) is the peak fluorescence wavelength; φ_{f} is the fluorescence quantum yield determined vs. PPa in toluene (φ_{f} = 0.3)²³ 5% error.; φ_{Δ} is the singlet oxygen quantum yield determined vs. PPa in toluene (0.5),²⁴ 10% error; τ_{Δ} is the singlet oxygen lifetime.

### Results

### Cellular Phototoxicity of PPa and Novel PPa-Derivatives on Tumour Cell Lines

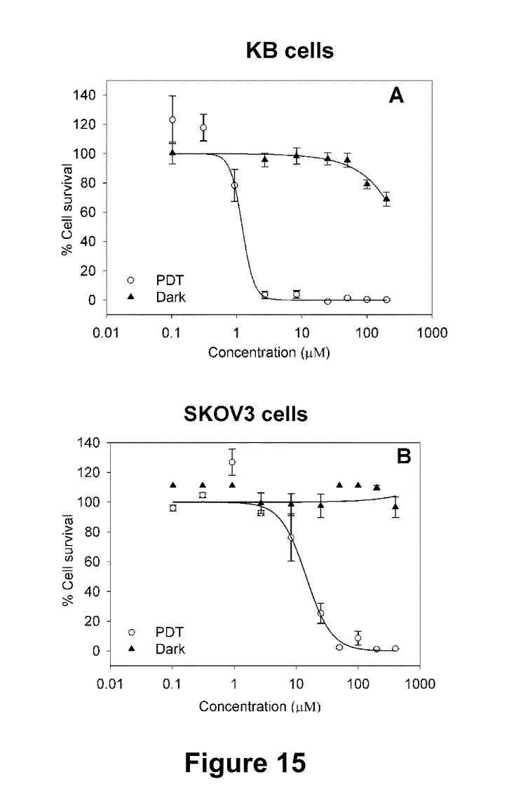

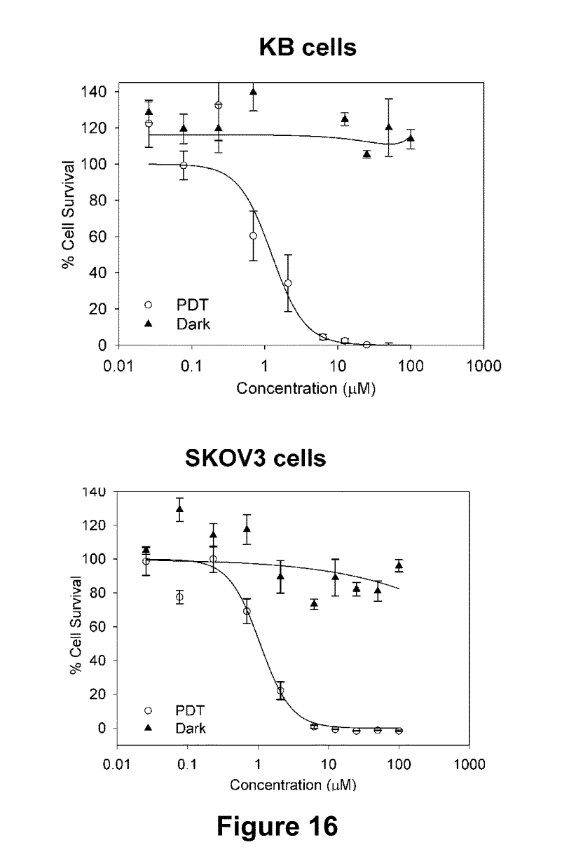

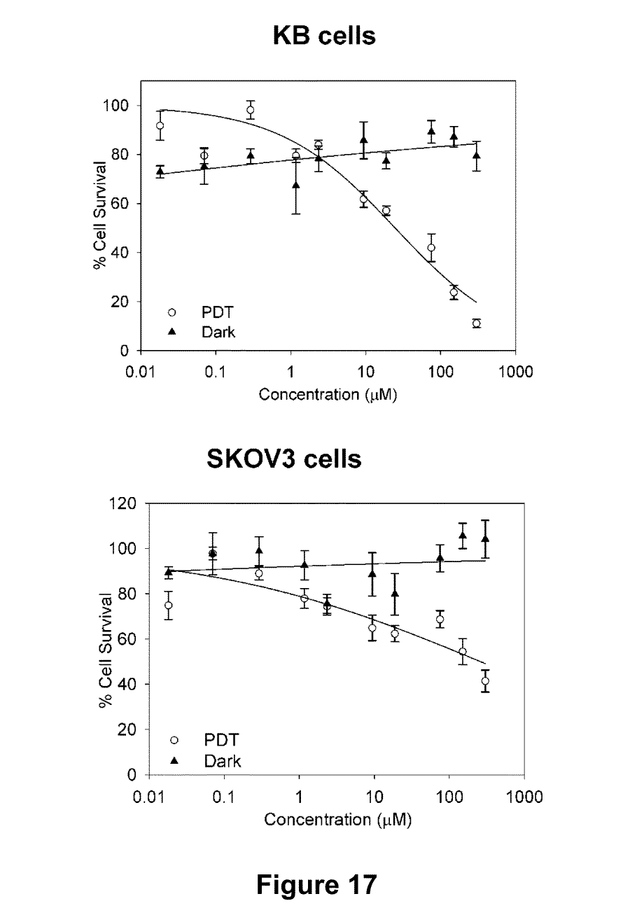

PPa is phototoxic to SKOV3 and KB tumour cell lines grown in culture, in a dose-dependent manner (FIG. 15). Two aqueously-soluble PPa derivatives examples, PPa-PEG1 (compound II) (FIG. 16) and cationic-PPa (compound 31C) (FIG. 17) are also phototoxic as stand-alone photosensitisers on SKOV3 and KB cell lines, with differing potencies. This shows that that modifying the physical-chemical properties does not lead to a loss of cell-killing function. Table 4 shows their IC50s. 11 is more potent than PPa.

TABLE 4
Summary of potencies (IC50s) of free photosensitisers.

| Compound | IC₅₀ (μM) on SKOV3 | IC₅₀ (μM) on KB |
|---|---|---|
| PPa | 14.5 ± 3.2 | 1.2 ± 0.4 |
| PPa-PEG1 (11) | 1.1 ± 0.2 | 1.2 ± 0.5 |
| Cationic PPa (31C) | 259.6 ± 31 | 24.7 ± 11.7 |

### Cellular Phototoxicity of Antibody-Targeted Compound 11 Photosensitiser on Tumour Cell Lines

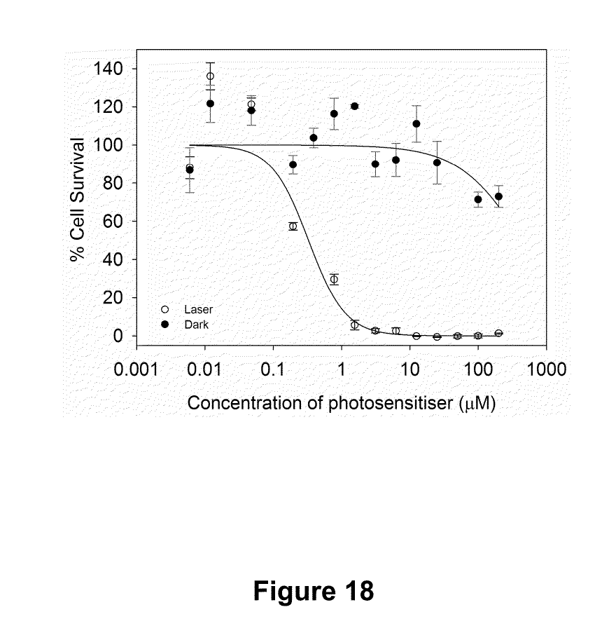

11 was further tested as a cell-targetable photo-immunoconjugate. An anti-HER2 scFv, C6.5 (Adams et al., 2000) was expressed and purified as described in the methods (Bhatti et al, 2007) and conjugated to 11 as described in the methods. The results are shown in FIG. 18. 11 was seen to be almost 3-times more potent to SKOV3 tumour cells due to the HER2 targeting (IC50 improved from 1.1 μM to 0.3 μM). The IC50 for HER2 targeted PPa (Bhatti et al, 2007) was around 7 mM. Therefore targeted soluble derivatives of PPa are more potent than targeted PPa, in this example, more than 20-fold more potent.

### Intracellular Localisation of PPa and Novel PPa-Derivatives in the SKOV3 Tumour Cell Line

### (a) Modification of the Physical-Chemical Properties of PPa Can Alter the Intracellular Targeting and Localisation Photosensitisers.

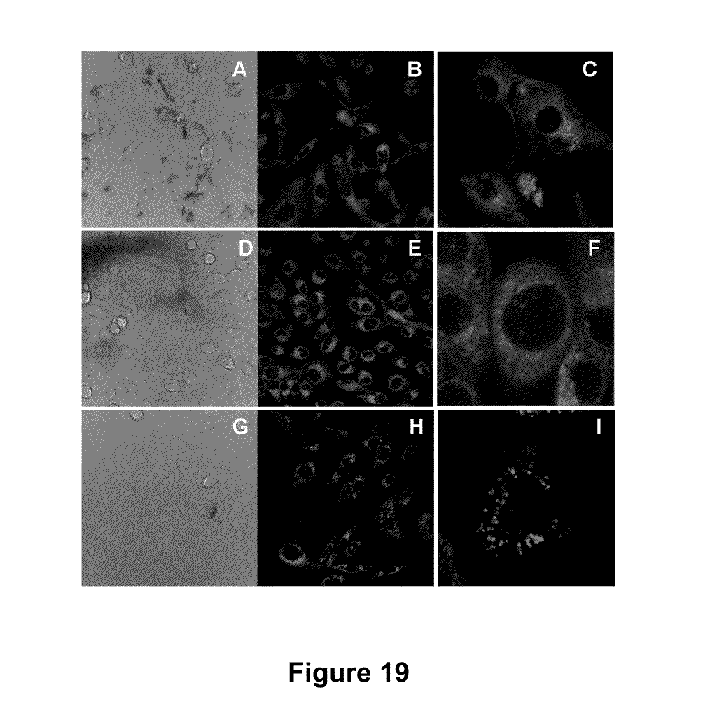

PPa, like many lipophillic photosensitisers localises in membrane-rich organelles (Macdonald et al, 1999) such as the mitochondria, endoplasmic reticulum, golgi and to a lesser extent in lysosomes, which are more aqueous vesicles. The literature suggests that membrane-vesicle localisation, particularly the mitochondria and ER lead to more potent PDT function as these organelles are more sensitive to reactive-oxygen species damage and subsequent cell death (refs 108, 111 and 107). FIG. 19 shows the localisation of PPa compared to two examples, 11 and 31C. The inherent fluorescence of the photosensitisers is being followed. PPa and 11 show similar membrane-rich organelle localisation, as indicated by the diffuse intracellular staining, with pockets of intense staining. 31C, which is positively charged, has a more punctuate staining pattern indicative of endosomal (aqueous compartment) localisation.

### (b) Modification of the Physical-Chemical Properties of PPa Can Alter the Golgi Localisation/Targeting of Photosensitisers.

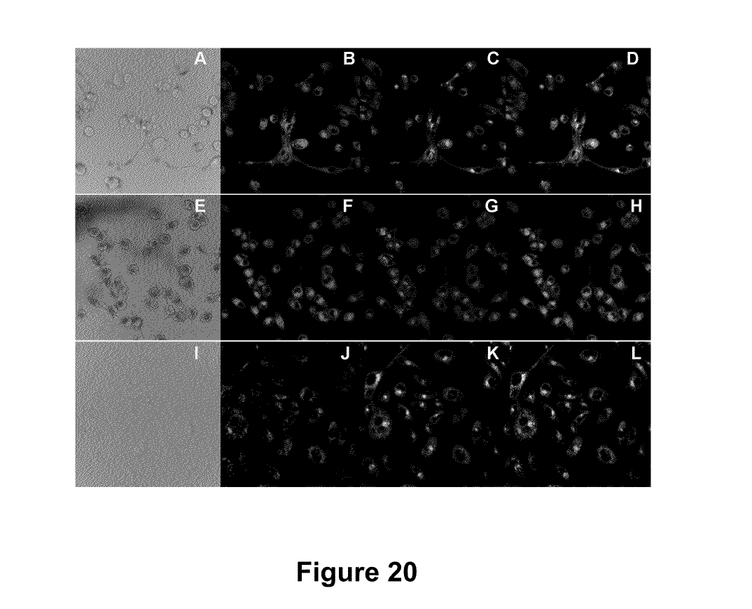

The golgi vesicle network is membrane-rich and contains many dynamic segments. The photosensitisers localisation was followed by measuring its intrinsic fluorescence (seen as red in FIG. 20). Simultaneously, Bodipy ceramide dye was used to counterstain the tumour cells. This dye is an established marker for golgi organelles and is seen as green in FIG. 20. Co-localisation studies show significant yellow staining for PPa and 11, indicating golgi localisation, whereas the positively-charged 31C shows very little yellow staining, indication very low localisation to the golgi network (FIG. 20).

### (c) Modification of the Physical-Chemical Properties of PPa Can Alter the Mitochondrial Localisation/Targeting of Photosensitisers.

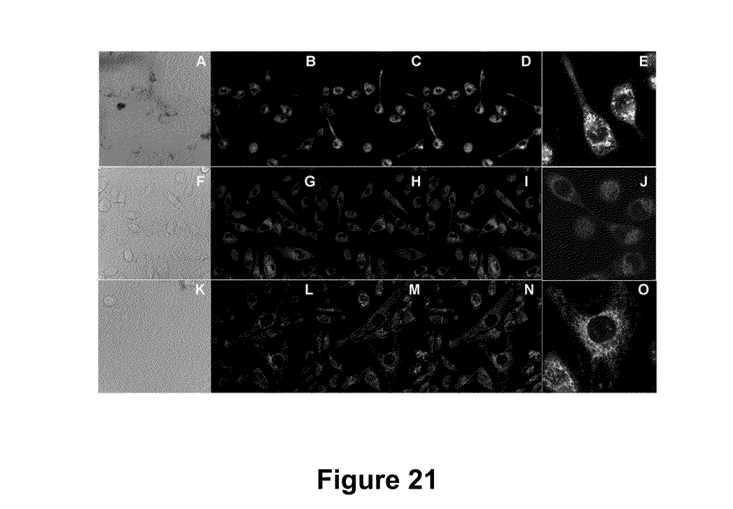

Mitochondria organelles are highly membrane-rich components and often regarded at the initiating centre for cellular apoptosis via the intrinsic pathway. The photosensitisers localisation was followed by measuring its intrinsic fluorescence (seen as red in FIG. 21). Simultaneously, Mito-tracker dye was used to counter-stain the tumour cells. This dye is an established marker for mitochondria organelles and is seen as green in FIG. 21. Co-localisation studies show significant yellow staining for PPa and 11, indicating significant mitochondrial localisation, whereas the positively-charged 31C shows very little yellow staining, indication very low localisation to the mitochondria (FIG. 21).

### (d) Modification of the Physical-Chemical Properties of PPa Can Alter the Lysosomal Localisation/Targeting of Photosensitisers.

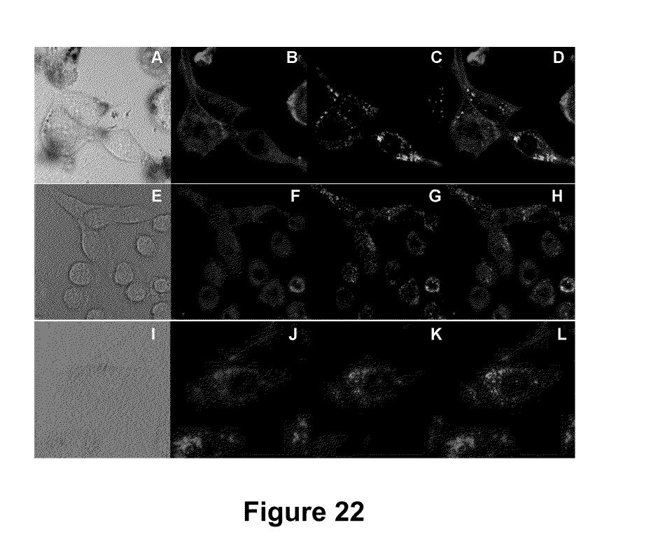

Lysozome organelles are membrane-poor components of the cell, containing aqueous compartments for proteolytic degradation. The photosensitisers localisation was followed by measuring its intrinsic fluorescence (seen as red in FIG. 22). Simultaneously, Lyso-tracker dye was used to counterstain the tumour cells. This dye is an established marker for lysosome organelles and is seen as green in FIG. 22.

Co-localisation studies show significant yellow staining for 31C, indicating significant lysosomal localisation. This was expected for a very soluble, charged photosensitiser. To a lesser extent, the neutral but more aqueously soluble 11 photosensitiser shows some yellow staining, also indicating localisation to the lysosomes (FIG. 22). PPa is very lipophillic and shows insignificant yellow staining, indicating insignificant lysosomal localisation (FIG. 22).

### (e) Modification of the Physical-Chemical Properties of PPa Can Alter the Endoplasmic Reticulum Localisation/Targeting of Photosensitisers.

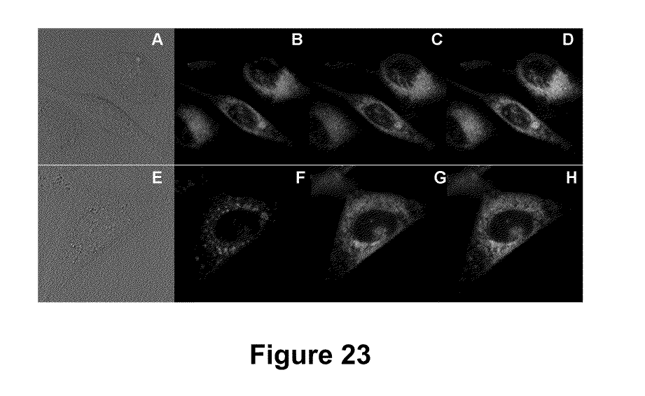

The endoplasmic reticulum (ER) organelles network is a highly membrane-rich component. The photosensitisers localisation was followed by measuring its intrinsic fluorescence (seen as red in FIG. 23). Simultaneously, ER-tracker dye was used to counter-stain the tumour cells. This dye is an established marker for the ER organelles and is seen as green in FIG. 23.

Co-localisation studies show significant yellow staining for 11, indicating significant ER localisation, whereas the positively-charged 31C shows no yellow staining, indication no localisation to the ER (FIG. 23).

### Increased Solubility of Photosensitisers Leads to Reduced Skin Photosensitivity

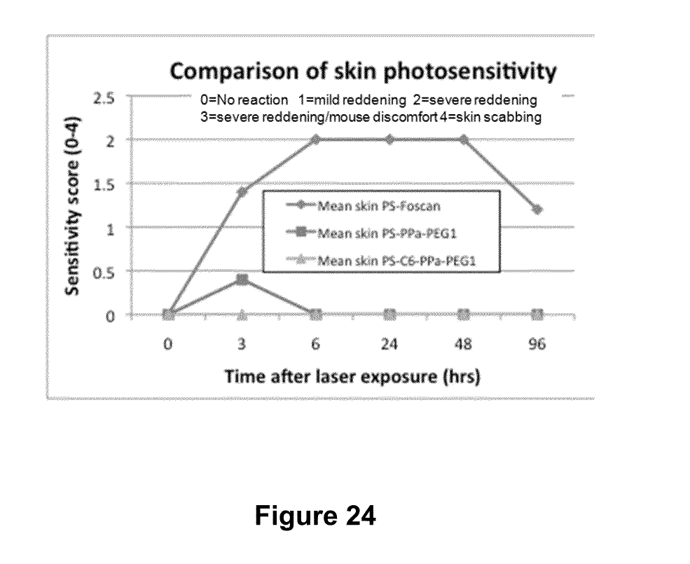

Skin photosensitivity is an important issue to be resolved in PDT. Established compounds such as Foscan demonstrates significant skin photosensitivity (ref 109). In a murine model, Foscan was administered at clinical doses (ref 109) and compared to compound 11 and a photo-immunoconjugate of compound 11. Skin photosensitivity was monitored over 4 days by observation according to a scale of 1-4 (FIG. 24). Foscan was seen to cause significant skin sensitivity whereas 11 and its conjugate showed little or no skin sensitivity (FIG. 24). This results suggests that our novel derivatives of PPa have improved pre-clinical properties.

### REFERENCES

[1] Price & Sikora (eds). Treatment of Cancer. Chapman & Hall 1995
[2] Press, OW & Rasey, J (2000). Semin Oncol. 6, 62-73. Priciples of Radioimmunotherapy for hematologists and oncologists
[3] Affleck, K et al (1992). Br. J. Cancer 65, 838-844. Monoclonal antibody targeting of methotrexate (MTX) against MTX-resistant tumour cell lines.
[4] Beers R et al. (2000). Clin Cancer Res. 6, 2835-43. Immunotoxins with increased activity against epidermal growth factor receptor vIll-expressing cells produced by antibody phage display.
[5] Rosenkranz, A. A et al (2000). Immunol. And Cell Biol. 78, 452-64. Targeted intracellular delivery of photosensitizers to enhance photodynamic therapy.
[6] Dolmans et al. (2003). Nature Rev. Cancer 3, 380-386. Photodynamic therapy for cancer.
[7] Hudson P J. (2000). Expert Opin Investig Drugs 9, 1231-42
Recombinant antibodies: a novel approach to cancer diagnosis and therapy.
[8] Borsi L, Balza E, Carnemolla B, Sassi F, Castellani P, Berndt A, Kosmehl H, Biro A, Siri A, Orecchia P, Grassi J, Neri D & Zardi L (2003) Blood. 102, 4384-92. Selective targeted delivery of TNFalpha to tumor blood vessels.
[9] Kuby (2000). Immunology, 4^{th} Ed. W. H. Freeman.
[10] Hoogenboom H R. Selecting and screening recombinant antibody libraries. Nature Biotechnology (2005) 23, 1105-16
[11] Milenic D E, Brady E D & Brechbiel M W (2004) Nat Rev Drug Discov. 3, 488-99. Antibody-targeted radiation cancer therapy.
[12] Harris M (2004) Lancet Oncol. 5, 292-302. Monoclonal antibodies as therapeutic agents for cancer.
[13] Wu A M and senter PD. Arming antibodies: Prospects and challenges for Immunoconjugates Nature Biotechnology (2005) 23, 1137-46 Improving the efficacy of antibody-based cancer therapies.
[14] Deonarain, M P, Rowlinson-Busza, G, George, A J & Epenetos, A A, (1997) Protein Eng. 10, 89-98. Redesigned anti-human placental alkaline phosphatase single-chain Fv: soluble expression, characterization and in vivo tumour targeting.
[15] Batra S K, Jain M, Wittel U A, Chauhan S C & Colcher D (2002) Curr Opin Biotechnol. 13, 603-8. Pharmacokinetics and biodistribution of genetically engineered antibodies.
[16] Boxer G M et al. (1994). Br. J. Cancer 69, 307-14. Localisation of monoclonal antibodies reacting with different epitopes on carcinoembryonic antigen (CEA)—implications for targeted therapy.
[17] Little M, Kyprianov S M, LeGall F & Moldenhauer G (2000). Immunol Today 21, 364-70. Of mice and men: hybridoma and recombinant antibodies.
[18] Verhaar M J et al. (1995). Int J Cancer 61, 497-501 A single chain Fv derived from a filamentous phage library has distinct tumor targeting advantages over one derived from a hybridoma.
[19] Begent, R H et al. (1996). Nat. Med. 2, 979-84 Clinical evidence of efficient tumor targeting based on single-chain Fv antibody selected from a combinatorial library.
[20] Epenetos A A, Snook D, Durbin H, Johnson P M, Taylor-Papadimitriou (1986). Cancer Res. 46, 3183-91. Limitations of radiolabeled monoclonal antibodies for localization of human neoplasms.
[21] Gangopadhyay, A et al. (1996). Nucl. Med. Biol. 23, 257-61 Modification of antibody isoelectric point affects biodistribution of 111-indium-labeled antibody.
[22] Chen S Y et al. (1995). Gene Ther. 2, 116-23. Design of a genetic immunotoxin to eliminate toxin immunogenicity.
[23] Deonarain M P & Epenetos A A (1998) Br. J. Cancer. 77, 537-46 Design, characterization and anti-tumour cytotoxicity of a panel of recombinant, mammalian ribonuclease-based immunotoxins.
[24] Linardou, H. et al. (2000) Int. J. Cancer 86, 561-569 A recombinant cytotoxic chimera based on mammalian deoxyribonuclease-
[25] Bonifacino J S & Traub L M (2003) Annu Rev Biochem. 72, 395-447. Signals for sorting of transmembrane proteins to endosomes and lysosomes.
[26] Ghettie, V. & Vitetta, E. (1994) Pharmacol. Ther. 63, 209-34. Immunotoxins in the therapy of cancer: from bench to clinic.
[27] Ancey C, Kuster A, Haan S, Herrmann A, Heinrich P C & Muller-Newen G (2003) J. Biol. Chem. 278, 16968-72. A fusion protein of the gp130 and interleukin-6Ralpha ligand-binding domains acts as a potent interleukin-6 inhibitor.
[28] Korner M, Waser B & Reubi J C (2005) Int J Cancer 115, 734-41. Neuropeptide Y receptors in renal cell carcinomas and nephroblastomas.
[29] Koide A, Bailey C W, Huang X & Koide S (1998) J Mol. Biol. 284, 1141-51. The fibronectin type III domain as a scaffold for novel binding proteins.
[30] Holt L J, Herring C, Jespers L S, Woolven B P & Tomlinson I M (2003) Trends Biotechnol. 21, 484-90. Domain antibodies: proteins for therapy.
[31] Schlehuber S & Skerra A (2001) Biol. Chem. 382, 1335-42. Duocalins: engineered ligand-binding proteins with dual specificity derived from the lipocalin fold.
[32] Binz H K, Amstutz P, Kohl A, Stumpp M T, Briand C, Forrer P, Grutter M G & Pluckthun A (2004) Nat. Biotechnol. 22, 575-82. High-affinity binders selected from designed ankyrin repeat protein libraries.
[33] Vasserot, A P et al (2003) Drug Discovery Today 8, 118-126. Optimization of protein therapeutics by directed evolution
[34] Hopper, C. (2000). Lancet Oncology 1, 212-219. Photodynamic therapy: a clinical reality in the treatment of cancer.
[35] Brown, S, Brown, E & Walker, I (2004) Lancet Oncology 5, 497-508. The present and future of photodynamic therapy in cancer treatment.
[36] Schmidt-Erfurth U. et al. (1999). Arch. Ophthalmol. 117, 1329-1345. Treatment of Age-related Macular degeneration with photodynamic therapy (TAP) study group. Photodynamic therapy of subfoveal neovascularization in age-related macular degeneration with verteporfin.
[37] Leman J A & Morton C A (2002) Expert Opin Biol Ther. 2, 45-53. Photodynamic therapy: applications in dermatology.
[38] Yamaguchi A, Woodburn K W, Hayase M, Hoyt G, Robbins R C. (2001). Transplantation 71, 1526-32. Photodynamic therapy with motexafin lutetium (Lu-Tex) reduces experimental graft coronary artery disease.
[39] Pfitzner A, Sigusch B W, Albrecht V, Glockmann E. (2004). J. Periodontol. 75, 1343-9. Killing of periodontopathogenic bacteria by photodynamic therapy.
[40] Demidova T N, Hamblin M R. (2004). Int J Immunopathol Pharmacol. 17, 245-54. Photodynamic therapy targeted to pathogens.
[41] Polo, L. et al. (1992). Cancer Letts 66, 217-23. The distribution of the tumour photosensitizers Zn(II)-phthalocyanine and Sn (IV)-etiopurpurin among rabbit plasma proteins.
[42] Moan J and Berg K (1992). Photochemotherapy of cancer-experimental research. Photochem. Photobiol. 55, 931-948
[43] Kessel D et al (2003). Localization and photodynamic efficacy of two cationic porphyins varying in charge distributions. Photochem. photobiol. 78, 431-435
[44] Dellinger, M et al. (1996). Photochem. Photobiol. 64, 182-7 Apoptosis or necrosis following Photfrin photosensitization: Influence of the incubation protocol.
[45] Ahmad, N. et al (1998). PNAS USA 95, 6977-6982. Photodynamic therapy results in induction of WAF1/CIP1/P21 leading to cell cycle arrest and apoptosis.
[46] Fiers, W. et al. (1998). Oncogene 18, 7719-7730. More than one way to die: apoptosis, necrosis and reactive oxygen species.
[47] Koudinova et al (2003) Int J. Cancer 104, 782-789. TOOKAD (Pd-bacteriopheophorbide)
[48] Dolmans D E, Kadambi A, Hill J S, Flores K R, Gerber J N, Walker J P, Rinkes I H, Jain R K, Fukumura D. (2002). Cancer Res. 62, 4289-94. Targeting tumor vasculature and cancer cells in orthotopic breast tumor by fractionated photosensitizer dosing photodynamic therapy.
[49] Melnikova V O, Bezdetnaya L N, Brault D, Potapenko A Y, Guillemin F. (2000). Int J Cancer 88, 798-803. Enhancement of meta-tetrahydroxyphenylchlorin-sensitized photodynamic treatment on human tumor xenografts using a water-soluble vitamin E analogue, Trolox.
[50] Westerman et al. (1998) Int J. Cancer 76, 842-50. Long circulating halfOlife and high tumor selectivity of the photosensitizermeta tetrahydroxyphenylchlorin conjugated to polyethylene glycol in nude mice grafted with a human colon carcinoma
[51] van Nostrum (2004) Advances in Drug Delivery Rev. 56, 9-16. Polymeric micelles as carriers.
[52] Nordquist, R E & Chen W E (1996) WO9631237. Cancer treatment by photodynamic therapy in combination with an immunoadjuvant.
[53] Meade C & Hyde C (2004) Br. J. Opthal. 88, 212-217. Photodynamic therapy with verteporfin is effective, but how big is its effect? Results of a systematic review.
[54] Wyss P, Schwarz V, Dobler-Girdziunaite D, Hornung R, Walt H, Degen A, Fehr M. (2001) Int J Cancer 93, 720-24. Photodynamic therapy of locoregional breast cancer recurrences using a chlorin-type photosensitizer.
[55] Cuenca R E, Allison R R, Sibata C, Downie G H. (2004) Annals Surg. Oncol. 11, 322-27. Breast cancer with chest wall progression: treatment with photodynamic therapy
[56] Lou P J, Jager H R, Jones L, Theodossy T, Bown S G, Hopper C (2004). Br J. Cancer. 91, 441-6. Interstitial photodynamic therapy as salvage treatment for recurrent head and neck cancer.
[57] Jager H R, Taylor M N, Theodossy T, Hopper C. (2005). MR imaging-guided interstitial photodynamic laser therapy for advanced head and neck tumors. AJNR Am J. Neuroradiol. 26, 1193-200.
[58] Wagnieres G, Hadjur C, Grosjean P, Braichotte D, Savary J F, Monnier P & van den Bergh H (1998) Photochem Photobiol. 68, 382-7. Clinical evaluation of the cutaneous phototoxicity of 5,10,15,20-tetra(m-hydroxyphenyl)chlorin.
[59] Moriwaki S I, Misawa J, Yoshinari Y, Yamada I, Takigawa M, Tokura Y (2001) Photodermatol Photoimmunol Photomed. 17, 241-3. Analysis of photosensitivity in Japanese cancer-bearing patients receiving photodynamic therapy with porfimer sodium (Photofrin™).
[60] Murrer L H, Hebeda K M, Marijnissen J P & Star W M (1999) Br J Cancer 80, 744-55. Short- and long-term normal tissue damage with photodynamic therapy in pig trachea: a fluence-response pilot study comparing Photofrin™ and mTHPC.
[61] Baas P, van Mansom I, van Tinteren H, Stewart F A, van Zandwijk N (1995) Lasers Surg Med. 16, 359-67. Effect of N-acetylcysteine on Photofrin™-induced skin photosensitivity in patients.
[62] Nseyo U O, Shumaker B, Klein E A, Sutherland K (1998) J. Urol. 160, 39-44. Photodynamic therapy using porfimer sodium as an alternative to cystectomy in patients with refractory transitional cell carcinoma in situ of the bladder.
[63] Vrouenraets, M B, Visser G W, Stewart F A, Stigter M, Oppelaar H, Postmus P E, Snow G B, van Dongen G A. (1999). Cancer Res. 59, 1505-1513. Development of meta-tetrahydroxypheneylchlorin-monoclonal antibody conjugates for photoimmunotherapy.
[64] Vrouenraets, M B, Visser G W, Loup C, Meunier B, Stigter M, Oppelaar H, Stewart F A, Snow G B, van Dongen G A. (2000). Int. J. Cancer 88, 108-114. Targeting of a hydrophilic photosensitizer by use of internalizing monoclonal antibodies: A new possibility for use in photodynamic therapy.
[65] van Dougen G A M S, Visser G W M & Vrouenraets, M B (2004). Adv. Drug Del. Rev 56, 31-52. Photosensitizer-antibody conjugates for detection and therapy of cancer.
[66] Vrouenraets, M B, Visser G W M, Stigter M, Oppelaar H, Snow G & van Dongen G A M S (2001). Targeting pf aluminium (III) phthalocyanine tetrasulfonate by use of internalizing monoclonal antibodies: Improved efficacy in photodynamic therapy.
[67] Hudson, R, et al (2005). Br. J. Cancer 92, 1442-1449. The development and characterisation of porphyrin isothiocyanate-monoclonal antibody conjugates for photo-immunotherapy.
[68] Savellano M D, Pogue B W, Hoopes P J, Vitetta E S & Paulsen K D (2005). Cancer Res. 65, 6371-9. Multiepitope Her2 targeting enhances photoimmunotherapy of Her2 expressing cancer cells with pyropheophorbide-a immunoconjugates.
[69] Savellano M D & Hasan T (2005). Clin. Cancer. Res 11, 1658-1668. Photochemical targeting of epidermal growth factor receptor: a mechanistic study.
[70] Birchler M, Viti F, Zardi L, Spiess B, Neri D (1999). Nat. Biotechnol. 17, 984-8. Selective targeting and photocoagulation of ocular angiogenesis mediated by a phage-derived human antibody fragment.
[71] Roder B & Hackbarth S (2001). WO0108704. Dendrimer-photosensitizer complexes for medical applications.
[72] Westerman P, Glanzmann T, Andrejevic S, Braichotte D R, Forrer M, Wagnieres G A, Monnier P, van den Bergh H, Mach J P, Folli S (1998) Int J Cancer 76, 842-50. Long circulating half-life and high tumor selectivity of the photosensitizer meta-tetrahydroxyphenylchlorin conjugated to polyethylene glycol in nude mice grafted with a human colon carcinoma.
[73] Demidova T N, Hamblin M R (2004) Int J Immunopathol Pharmacol. 17, 245-5. Photodynamic therapy targeted to pathogens.
[74] Deonarain M P & Stafford S (2003). WO03015825. Conjugate
[75] Glickman R D, Mayo G L, Mckinnon S J, Melendez R E & Kumar N C (2004). WO 080284 A2. Antibody targeted photodynamic therapy
[76] Mayo G L, Melendez R F, Kumar N, McKinnon S J & Glickman R D (2003). Am. J. Opthalmol. 136, 1151-2. Antibody-tageted photodynamic therapy
[77] Hasan T, Savellano M D & Skobe M (2002) WO 02100326. Photoimmunotherapies for cancer using photosensitizer immuno-conjugates and combination therapies
[78] Akhlynina T V, Jans D A, Rosenkranz A A, Statsyuk N V, Balashova I Y, Toth G, Pavo I, Rubin A B, Sobolev A S (1997). J. Biol. Chem. 272, 20328-20331. Nuclear targeting of chlorin e6 enhances its photosensitizing activity.
[79] Cavanaugh P G (2002) Breast Cancer Res Treat. 72, 117-30. Synthesis of chlorin e6-transferrin and demonstration of its light-dependent in vitro breast cancer cell killing ability.
[80] Cavanaugh P G (2002) US 20021337901. Synthesis and photodynamic therapy-mediated anti-cancer and other used of chlorin e6-transferrin.
[81] Khadem J, Veloso A A Jr, Tolentino F, Hasan T, Hamblin M R (1999). Invest Ophthalmol V is Sci. 40, 3132-7. Photodynamic tissue adhesion with chlorin(e6) protein conjugates.
[82] Green A M (2002). WO 02/080754 A2. Methods for using annexin for detecting cell death in vivo and treating associate conditions.
[83] Storrie B, Tarrago-Trani S (2002). B/B-like fragment for the purposes of photodynamic therapy and medical imaging WO 02/067850
[84] Ray R & Mohr S. (2001) WO 0178606. Selective nuclear receptor-targeted systems for delivery of cytotoxins to cancer cells for targeted photodynamic therapy
[85] Gaboury L & Villeneuve L (1996) U.S. Pat. No. 5,556,992. Novel rhodamine derivatives for photodynamic therapy of cancer and in vitro purging of the leukemias
[86] Schneider R, Schmitt F, Frochot C, Fort Y, Lourette N, Guillemin F, Muller J F, Barberi-Heyob M. (2005). Bioorgan. Med. chem. 13, 2799-2808. Design, synthesis, and biological evaluation of folic acid targeted tetraphenylporphyrin as novel photosensitizers for selective photodynamic therapy.
[87] Lutsenko S V, Feldman N B, Finakova G V, Posypanova G A, Severin S E, Skryabin K G, Kirpichnikov M P, Lukyanets E A, Vorozhtsov G N (1999). Tumour Biol. 20, 218-24. Targeting phthalocyanines to tumor cells using epidermal growth factor conjugates.
[88] Renno R Z, Terada Y, Haddadin M J, Michaud N A, Gragoudas E S, Miller J W. (2004). Arch Opthalmol. 122, 1002-1011. Selective photodynamic therapy by targeted verteporfin delivery to experimental choroidal neovascularization mediated by a homing peptide to vascular endothelial growth factor receptor-2.
[89] Boehm M K, Corper A L, Wan T, Sohi M K, Sutton B J, Thornton J D, Keep P A, Chester K A, Begent R H, Perkins S J. (2000). Crystal structure of the anti-(carcinoembryonic antigen) single-chain Fv antibody MFE-23 and a model for antigen binding based on intermolecular contacts. Biochem J. 346, 519-28.
[90] Hoogenboom H R, Griffiths A D, Johnson K S, Chiswell D J, Hudson P & Winter G (1991) Nucleic Acids Res. 19, 4133-7. Multi-subunit proteins on the surface of filamentous phage: methodologies for displaying antibody (Fab) heavy and light chains.
[91] Enever C, Tomlinson I M, Lund J, Levens M & Holliger P (2005) J. Mol. Biol. 347, 107-20. Engineering high affinity superantigens by phage display.
[92] Li Y, Moysey R, Molloy P E, Vuidepot A L, Mahon T, Baston E, Dunn S, Liddy N, Jacob J, Jakobsen B K & Boulter J M (2005) Nat. Biotechnol. 23, 349-54. Directed evolution of human T-cell receptors with picomolar affinities by phage display.
[93] Embleton M L, Nair S P, Cookson B D & Wilson M (2002) J. Antimicrob. Chemother. 50, 857-864. Selective lethal photosensitization of methicillin-resistant Staphylococcus aureus using an IgG-tin(IV) chlorin e6 conjugate
[94] Garcia D I & Yahioglu, G (WO2004/046151). Porphyrin derivatives
[95] Sokous N S, Hamblin M R, Deutsch T F & Hasan T (2001). Monoclonal antibody tagged receptor targeted contrast agents for detection of cancer. Proceedings of SPIE, 4259, 115. Biomarkers and Biological Spectral imaging, Eds. Bearman G H, Levenson R M and Bornhop D J.
[96] Kim J I, Wang C, Kuizon S, Xu J, Barengolts D, Gray P C, Rubenstein R (2005). Simple and specific detection of abnormal prion protein by a magnetic bead-based immunoassay coupled with laser-induced fluorescence spectrofluorometry. J Neuroimmunol 158, 112-9.
[97] Pasqualini R, Koivunen E, Ruoslahti E. (1997). Alpha v integrins as receptors for tumor targeting by circulating ligands. Nat. Biotechnol. 15, 542-6.
[98] Rusckowski M, Qu T, Chang F, Hnatowich D J. (1997). Technetium-99m labeled epidermal growth factor-tumor imaging in mice. J Pept Res. 50, 393-401.
[99] Weissleder R, Tung C H, Mahmood U, Bogdanov A Jr. (1999). In vivo imaging of tumors with protease-activated near-infrared fluorescent probes. Nat. Biotechnol. 17, 375-8.
[100] Pericleous, L M, Richards, J, Epenetos, A A, Courtenay-Luck, N & Deonarain M P (2005). Characterisation and internalization of recombinant humanized HMFG1 antibodies against MUC1. Br J. Cancer. 93 pp 1257-66
[101] Ward E S. Antibody engineering: the use of Escherichia coli as an expression host. FASEB J. 1992; 6: 2422-7.
[102] A. Osuka, Y. Wada and S. Shinoda. Covalently linked pyrophephorbide dimmers as models of the special pair in the photosynthetic reaction centre Tetrahedron, 1996, 52(12), 4311-4326.
[103] S-I. Sasaki, K. Mizutani, M. Kunieda and H. Tamiaki. Synthesis, modification, and optical properties of C3-ethynylated chlorophyll derivatives. Tet. Lett. 2008, 49, 4113-4115.
[104] R. K. Pandey, M. Isaac, I. Macdonald, C. J. Medforth, M. O, Senge, T. J. Dougherty and K. M. Smith. Pinacol-pinacolone rearrangements in vic-dihydroxychlorins and bacteriochlorins. Effects of substituents at the peripheral positions. J. Org. Chem. 1997, 62(5), 1463-1472.
[105] Y. Saito, K. Matsumoto, S. S. Bag, S. Ogasawara, K. Fujimoto, K. Hanawa and I. Saito. C8-alkynyl- and alkylamino substituted 2′-deoxyguanosines: a universal linker for nucleic acids modification.Tetrahedron, 2008, 64(16), 3578-3588.
[106] Adams G P, Shaller C C, Chappell L L, Wu C, Horak E M, Simmons H H, Litwin S, Marks J D, Weiner L M, Brechbiel M W. Delivery of the alpha-emitting radioisotope bismuth-213 to solid tumors via single-chain Fv and diabody molecules. Nucl Med. Biol. 2000, 27, 339-46.
[107] Bonneau S and Vever-Bizer C, Tetrapyrrole photosensitisers, determinants of subcellular localisation and mechanisms of photodynamic processes in therapeutic approaches, Expert Opin. Ther. Patents, 2008, 18 (9), 1011-25
[108] Castano A P, Demidova T D & Hamblin M R. Mechanisms in photodynamic therapy: part one-photosensitisers, photochemistry and cellular localisation. Photodiagnosis and Photodynamic therapy 2004 1, 279-93
[109] Cramers P, Ruevekamp M, Oppelaar H, Dalesio O, Baas P & Stewart F A. Foscan uptake and tissue distribution in relation to photodynamic therapy. Br. J. Cancer 2003 88, 283-290
[110] Macdonald, I J, Morgan J, Bellnier D A, Paszkiewics G M, Whitaker J E, Litchfield D J and Dougherty T J, Subcellular localisation patterns and their relationship to photodynamic activity of pyropheophorbide-a derivatives. Photochem. Photobiol., 1999, 70, 789-797
[111] Oleinick N L, Morris R L and Belichenko T, The role of apoptosis in response to photodynamic therapy: what, where, why, and how. Photochem. Photobiol. Sci., 2002, 1, 1-21

1. A compound of Formula I: 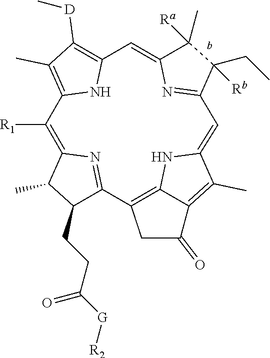
wherein:
when b represents a double bond, D represents —CH₂— or Q, R^{a} and R^{b} are both not present;
when b represents a single bond, D represents —C(O)—, —CH₂— or Q, R^{a} and R^{b} are either both H or both —OH;
G represents 0 or a direct bond;
Q represents a structural fragment of formula Ig or Ih, 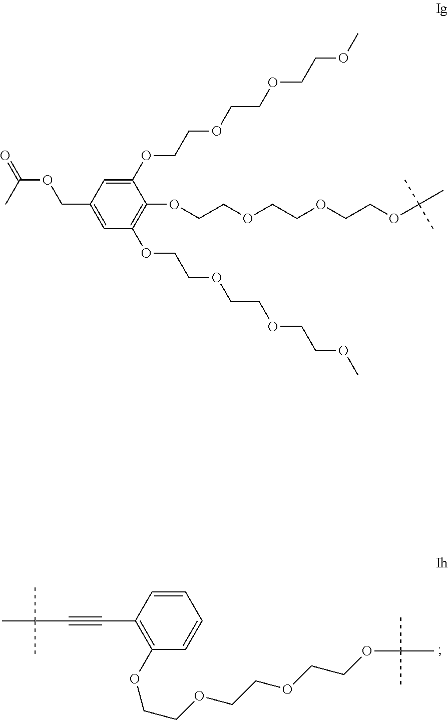
where either
(i) R₁ represents —(CH₂)ₐ—C(R₃)═C(R₄)—(CH₂)ᵥ—X, or —(CH₂)_{w}—C≡C—(CH₂)ₓ—X;
the sum of w and x is from 2 to 10;
the sum of u and v is from 2 to 6;
X represents —C(O)-L₁, —OH, —CN, —SH, —NHR₃ₐ, halo, phosphoramidityl, N-hydroxy succinimidyl ester, sulfo-N-hydroxy succinimidyl ester, fluorophenyl esters, isothiocyanato, iodoacetamidyl, maleimidyl, aryl or hetroaryl which latter two groups are substituted by one or more groups selected from —C(O)-L₁, —OH, a sulfonyl ester, —NO₂, —CHO, —N₃, —CN, —SH, —NHR₃ₐ, halo, phosphoramidityl, N-hydroxy succinimidyl ester, sulfo-N-hydroxy succinimidyl ester, fluorophenyl esters, isothiocyanato, iodoacetamidyl and maleimidyl;
L₁ represents —OH or —O—C(O)—R₅, or —C(O)-L₁ represents a carboxylic acid functional group activated by a carbodiimide;
R₂ represents alkyl, cycloalkyl, alkylenyl, alkynyl, aryl, benzyl, heteroaryl wherein the latter three groups are substituted by one or more groups selected from —OH, —NH₂ or a C1 to C6 alkyl substituted by one or more halo atoms independently substituted by one or more linear or branched oligo or poly-ethyleneoxy groups wherein the total number of oligo or poly-ethyleneoxy groups is from 2 to 100, or R₂ represents —NR₆(R₇) or —N(R₆ₐ)—(CH₂)—SO₃⁻E⁺;
R₃ to R₅ and R₃ₐ independently represent C1 to C6 alkyl or C1 to C6 alkyl substituted by one of more groups selected from —OH and halo
R₆ and R₇ independently represent —(CH₂)_{z}—NR₈(R₉) or —(CH₂)_{z}—N⁺R₈(R₉)(R₁₀)A⁻, provided that at least one of R₆ and R₇ is not H;
R₆ₐ represents H or C1 to C3 alkyl or C1 to C3 substituted by one or more groups selected from —OH or halo;
z represents 1 to 10;
R₈ to R₁₀ independently represents H, alkyl or alkenyl or either alkylor alkenyl substituted by one or more groups selected from —OH and halo;
A⁻ represents I⁻, Cl⁻ or Br⁻; and
E+ represents a cationic group;
(ii) R₁ represents a structural fragment of formula Ia, Ib, Ic, Id, Ie, If: 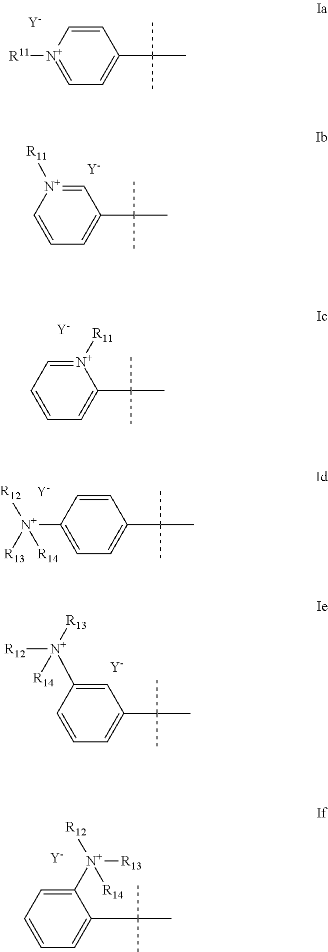
wherein the dashed lines indicate the point of attachment to the rest of the molecule, or
R₁ represents —(CH₂)ₜ—Z, —(CH₂)ᵤ—C(R₃)═C(R₄)—(CH₂)ᵥ—Z, or —(CH₂)_{w}—C≡C—(CH₂)ₓ—Z;
R¹¹ represents H, alkyl, alkyl substituted by one or more groups selected from —OH, halo or linear or branched ethyleneoxy groups wherein the total number of oligo or poly-ethyleneoxy groups is from 2 to 100;
R₁₂ to R₁₄ independently represent H or C1 to C6 alkyl or C1 to C6 alkyl substituted by one or more groups selected from —OH, halo and linear or branched ethyleneoxy groups wherein the total number of oligo or poly-ethyleneoxy groups is from 2 to 100;
r represents I⁻, Cl⁻, or Br⁻;
t represents 1 to 20;
the sum of w and x is from 2 to 6;
the sum of u and v is from 2 to 15;
Z represents —C(O)O⁻E⁺, —SO₃⁻E⁺, a quarternary ammonium salt, a structural fragment of formulae Ia to If, or Z represents aryl, benzyl, heteroaryl (wherein the latter three groups may be substituted by one or more groups selected from —OH, —NH₂ or a C1 to C6 alkyl substituted by one or more halo atoms) substituted by one or more —C(O)O⁻E⁺ groups, —SO₃⁻E⁺ groups, a quarternary ammonium salt, a pyridinium ion or linear or branched oligo or poly-ethyleneoxy groups wherein the total number of oligo or poly-ethyleneoxy groups is from 2 to 100;
E⁺ represents a cationic group;
R₂ represents —C(O)-L₃, phosphoramidityl, N-hydroxy succinimidyl ester, sulfo-N-hydroxy succinimidyl ester, fluorophenyl esters, isothiocyanato, iodoacetamidyl, maleimidyl;
L₃ represents —OH or —O—C(O)—R₁₅, halo, an activated ester such as 1-oxybenzotriazoyl or an aryloxy group optionally substituted with one or more subsistent selected from nitro, fluoro, chloro, cyano and trifluoromethyl, and
R₁₅ represents C1 to C6 alkyl or C1 to C6 alkyl substituted by one or more groups selected from —OH and halo;
or a pharmaceutically-acceptable salt or solvate thereof.

2. The compound of claim 1 wherein:
R₁ represents —(CH₂)_{w}—C≡C—(CH₂)ₓ—X;
the sum of w and x is from 2 to 10;
X represents —C(O)-L₁, phosphoramidityl, N-hydroxy succinimidyl ester, sulfo-N-hydroxy succinimidyl ester, fluorophenyl esters, isothiocyanato, iodoacetamidyl or maleimidyl;
L₁ represents —OH or —C(O)-L₁ represents a carboxylic acid functional group activated by a carbodiimide;
R₂ represents aryl, benzyl, heteroaryl (wherein the latter three groups may be substituted by one or more groups selected from —OH, —NH₂ or a C1 to C6 alkyl substituted by one or more halo atoms) independently substituted by one or more linear or branched oligo or poly-ethyleneoxy groups wherein the total number of oligo or poly-ethyleneoxy groups is from 2 to 100,
or R₂ represents —NR₆(R₇) or —N(R₆ₐ)—(CH₂)—SO₃⁻E⁺;
R₆ and R₇ independently represent —(CH₂)_{z}—NR₈(R₉) or —(CH₂)_{z}—N⁺R₈(R⁹)(R₁₀)A⁻, provided that at least one of R₆ and R₇ are not H;
R₆ₐ represents H;
z represents 1 to 10;
R₈ to R₁₀ independently represents H or alkyl optionally substituted by one or more groups selected from —OH or halo;
A⁻ represents I⁻, Cl⁻ or Br⁻, and
E+ represents a cationic group.

3. The compound of claim 1 wherein:
R₁ represents —(CH₂)_{w}—C≡C—(CH₂)ₓ—X;
the sum of w and x is from 2 to 10;
X represents —C(O)-L₁, phosphoramidityl, N-hydroxy succinimidyl ester, sulfo-N-hydroxy succinimidyl ester, fluorophenyl esters, isothiocyanato, iodoacetamidyl or maleimidyl;
L₁ represents —OH or —C(O)-L₁ represents a carboxylic acid functional group activated by a carbodiimide;
R₂ represents aryl, benzyl, heteroaryl wherein the latter three groups may be substituted by one or more groups selected from —OH, —NH₂ or a C1 to C6 alkyl substituted by one or more halo atoms independently substituted by one or more linear or branched oligo or poly-ethyleneoxy groups wherein the total number of oligo or poly-ethyleneoxy groups is from 2 to 100.

4. A compound according to claim 1 selected from compounds of formula IB to IG: 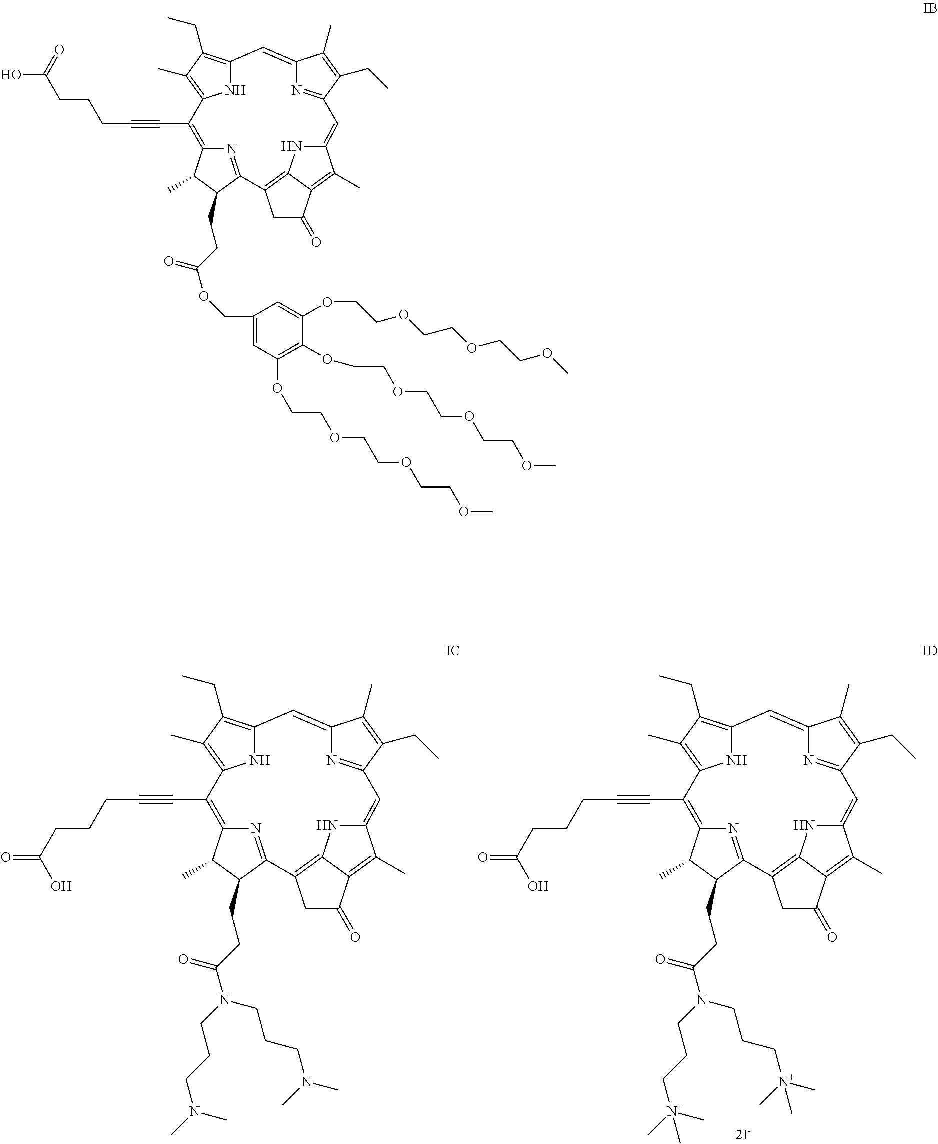 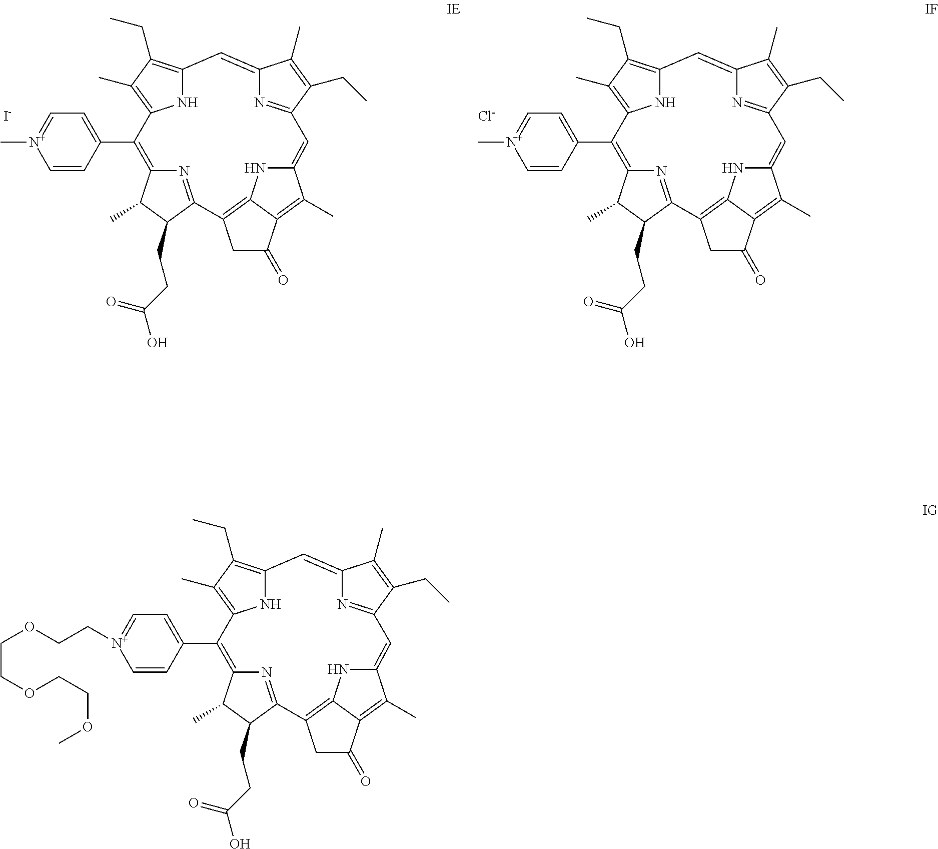
wherein E⁻ represents an anionic group.
